(19) United States
(12) Patent Application Publication
(10) Pub. No.: US 2019/0194628 A1
(43) Pub. Date: Jun. 27, 2019

(54) METHODS FOR PRODUCING VIRUS FOR VACCINE PRODUCTION

(71) Applicants: Takeda Vaccines, Inc., Cambridge, MA (US); Takeda Pharmaceutical Company Limited, Osaka (JP)

(72) Inventors: Raman RAO, Singapore (SG); Tatsuki SATO, Yamaguchi (JP); Kaori ODA, Yamaguchi (JP); Kuniaki NAKAMURA, Yamaguchi (JP); Takeshi NISHIHAMA, Yamaguchi (JP)

(73) Assignees: Takeda Vaccines, Inc., Cambridge, MA (US); Takeda Pharmaceutical Company Limited, Osaka, MA (US)

(21) Appl. No.: 16/329,654

(22) PCT Filed: Sep. 1, 2017
(86) PCT No.: PCT/US17/49956

§ 371 Date: Feb. 28, 2019

Related U.S. Application Data
(60) Provisional application No. 62/382,621, filed on Sep. 1, 2016.

(51) Int. Cl. C12N 7/00

(52) U.S. Cl. CPC C12N 7/00; C12N 2770/32634; C12N 2770/32651

(57) ABSTRACT

The present disclosure relates to methods of producing Enterovirus C, e.g., for poliomyelitis vaccine production. In some embodiments, the methods include adding polysorbate to the cell culture medium during or prior to inoculation with the virus and/or culturing cells in a fixed bed bioreactor. Further provided herein is an Enterovirus C produced by the methods of production disclosed herein, as well as compositions, immunogenic compositions, and vaccines related thereto.

### CROSS-REFERENCE TO RELATED APPLICATIONS

This application claims the benefit of U.S. Provisional Application No. 62/382,621, filed Sep. 1, 2016, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 606772001240SEQLIST.txt; date recorded: Aug. 24, 2017, size: 2 KB).

### FIELD

The present disclosure relates to methods for producing virus (e.g., Enterovirus C virus) for vaccine production.

### BACKGROUND

Poliomyelitis is caused by viral infection of human enterovirus C (HEV-C), which encompasses a variety of viral subtypes including several serotypes of poliovirus. Poliovirus is typically transmitted between humans via oral secretions or contact with fecal material from an infected individual. Most infections lead only to asymptomatic viral replication limited to the alimentary tract. However, in less than 1 percent of infections, the virus infects the central nervous system and replicates in the motor neurons of the anterior horn cells in the spinal cord, leading to acute flaccid paralysis and in some cases difficulty speaking, swallowing, breathing, and death. In the United States alone, tens of thousands of people were infected by poliomyelitis each year until 1955, when Jonas Salk developed an inactivated polio vaccine. Albert Sabin later developed an oral polio vaccine (OPV) using an attenuated virus. These have led to the near-eradication of polio infection in humans; however, 223 cases of paralytic polio were reported in 2012 (Sutter, R. W. et al. (2014) J. Infect. Dis. 210:S434-S438). Polio remains endemic in Nigeria, Afghanistan, and Pakistan, and sporadic outbreaks have occurred in various countries in the Middle East, Africa, and Asia.

Human enterovirus C belongs to the Picornaviridae family of non-enveloped, positive-sense RNA viruses, which also includes certain rhinoviruses and certain coxsackieviruses. Members of the HEV-C group include three poliovirus serotypes (S1, S2, and S3; also known as PV1, PV2, and PV3) and numerous Coxsackie A virus serotypes (e.g., CAV serotypes 1, 11, 13, 15, 17, 18, 19, 20, 21, 22, and 24) (Brown, B. et al. (2003) J. Virol. 7:8973-84). While wild poliovirus serotype 2 (WPV2) is considered eradicated (with the last known infection occurring in 1999), and WPV3 infection last occurred in 2012, but WPV1 infections were still being reported (Lowther, S. A. (2013) Morbidity and Mortality Weekly Report 62:335-8).

The Polioviruses contain an icosahedral capsid made up of 60 copies each of coat proteins VP1, VP2, VP3, and VP4 (Bubeck, D. et al. (2005) J. Virol. 7:7745-55). Virus binding to the specific poliovirus receptor (Pvr, also known as CD155) leads to cellular infection for all three viral serotypes. However, these serotypes contain serotype-specific and general immunodominant epitopes recognized by neutralizing antibodies (Minor, P. D. et al. (1986) J. Gen. Virol. 6:1283-91). The inactivated polio vaccine contains formalin-inactivated wild-type strains of each serotype. The Sabin oral polio vaccine contains a mixture of three live, attenuated poliovirus serotypes, but monovalent vaccines against each poliovirus serotype are also employed to reduce transmission of specific serotypes. However, these attenuated viruses have been shown to acquire neurovirulence and transmissibility, resulting in outbreaks due to circulating vaccine-derived poliovirus (cVDPV). Due to these outbreaks and the continued outbreaks of wild poliovirus due to incomplete eradication, the need exists for continued polio vaccine production.

Viral vaccine production on an industrial scale requires significant resources and cost. For production of potential vaccines, viral vaccines are usually produced by anchorage-dependent cell lines (e.g. VERO cells). At industrial scale, these cells are either cultivated in static mode on multi-plate systems (e.g., Cell Factories, Cell Cube, etc.), on roller bottles, or on microcarriers (porous or non-porous) in suspension in bioreactors. Multi-plate systems are bulky and require significant handling operations, whereas microcarrier cultures require numerous operations (sterilization and hydration of carriers, etc.) and many steps from precultures to final process with complex operations (i.e. bead-to-bead transfers).

Once the virus is produced, existing protocols for downstream purification are often burdensome and resource-intensive. For example, U.S. Pat. No. 8,753,646 describes a protocol for poliovirus purification that includes multiple filtration and ultracentrifugation steps before an eventual column-based purification of the virus. Each additional step requires manpower, time, and resources. Therefore, a need exists for vaccine production techniques that use a more streamlined downstream purification scheme, which would allow for a simple, shorter process that reduces footprint, manpower, and operational costs.

Polio vaccines are still needed in several regions of Africa and the Middle East where poliovirus remains endemic. However, these regions are economically disadvantaged, lacking sufficient resources and/or infrastructure for effective vaccination programs. Therefore, the eradication of polio requires more cost- and resource-efficient methods for virus production.

### BRIEF SUMMARY

Thus, there is a need to develop methods for Enterovirus C production, e.g., methods that allow for cost-effective viral production on an industrial scale. As disclosed herein, the methods of the present disclosure, inter alia, use a fixed bed culture system to enable enhanced viral production with greater cost efficiency and streamlined processing. For example, the improvements described herein are capable of reducing the per-dose cost of vaccine from approximately S5 to $1.25. In some embodiments, the methods of the present disclosure may additionally or alternatively include addition of polysorbate to the cell culture medium during or prior to inoculation of the cell with the virus. Advantageously, these methods may be used, for example, for large- or industrial-scale production of an Enterovirus C, which is useful in the manufacture of vaccines and/or immunogenic compositions.

Accordingly, certain aspects of the present disclosure provide method for producing an Enterovirus C virus, comprising: (a) culturing a cell in a first cell culture medium; (b) inoculating the cell with the Enterovirus C virus in a second cell culture medium under conditions in which the Enterovirus C virus infects the cell and the infected cell produces the Enterovirus C virus; and (c) harvesting the Enterovirus C virus produced by the cell, wherein a surfactant is added to the second cell culture medium during inoculation of the cell with the Enterovirus C virus or from approximately one hour to approximately four hours prior to step (c). In some embodiments, the yield of Enterovirus C virus harvested in step (c) is increased, as compared to a yield of Enterovirus C virus harvested in the absence of the surfactant. In some embodiments, the yield of Enterovirus C virus harvested in step (c) is increased by about 10%, about 20%, about 30%, about 40%, about 50%, about 60%, about 70%, about 80%, about 90%, about 100%, about two-fold, about three-fold, about four-fold, about five-fold, about six-fold, about seven-fold, about eight-fold, about nine-fold, or about ten-fold, as compared to a yield of Enterovirus C virus harvested in the absence of the surfactant. In some embodiments, the surfactant is a polysorbate. In some embodiments, the surfactant is a polyethylene glycol-based surfactant. Other aspects of the present disclosure provide method for producing an Enterovirus C virus, comprising: (a) culturing a cell in a first cell culture medium; (b) inoculating the cell with the Enterovirus C virus in a second cell culture medium under conditions in which the Enterovirus C virus infects the cell and the infected cell produces the Enterovirus C virus; and (c) harvesting the Enterovirus C virus produced by the cell, wherein dextran is added to the second cell culture medium during inoculation of the cell with the Enterovirus C virus or from approximately one hour to approximately four hours prior to step (c). In some embodiments, the yield of Enterovirus C virus harvested in step (c) is increased, as compared to a yield of Enterovirus C virus harvested in the absence of the dextran. In some embodiments, the yield of Enterovirus C virus harvested in step (c) is increased by about 10%, about 20%, about 30%, about 40%, about 50%, about 60%, about 70%, about 80%, about 90%, about 100%, about two-fold, about three-fold, about four-fold, about five-fold, about six-fold, about seven-fold, about eight-fold, about nine-fold, or about ten-fold, as compared to a yield of Enterovirus C virus harvested in the absence of the dextran. In some embodiments, the Enterovirus C virus is a poliovirus serotype selected from the group consisting of S1, S2, and S3. In some embodiments, the cell is cultured in step (a) in a liquid culture. In some embodiments, the cell is an adherent cell, and the cell is cultured in step (a) on a microcarrier. In some embodiments, the cell is an adherent cell, and the cell is cultured in step (a) in a fixed bed comprising a matrix. In some embodiments, the cell is cultured in step (a) in a bioreactor. In some embodiments, the cell is inoculated with the Enterovirus C virus at a multiplicity of infection (MOI) of between about 0.01 and about 0.0009. In some embodiments, between about 120,000 cells/cm² and about 300,000 cells/cm² are inoculated. In some embodiments, between about 4,000 cells/cm² and about 16,000 cells/cm² are inoculated. In some embodiments, about 5,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, no additional glucose is added to the second cell culture medium and/or glucose is depleted from the second culture medium. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus, wherein the cell is inoculated with the Enterovirus C virus at an MOI of between about 0.01 and about 0.0009; and (c) harvesting the Enterovirus C virus produced by the cell. In some embodiments, the Enterovirus C virus is a poliovirus serotype selected from the group consisting of S1, S2, and S3. In some embodiments, polysorbate is added to the second cell culture medium during inoculation of the cell with the Enterovirus C virus or from approximately one hour to approximately four hours prior to step (c). In some embodiments, between about 120,000 cells/cm² and about 300,000 cells/cm² are inoculated. In some embodiments, between about 4,000 cells/cm² and about 16,000 cells/cm² are inoculated. In some embodiments, about 5,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, the cell is inoculated at a pH that ranges from about 6.8 to about 7.4. In some embodiments, no additional glucose is added to the second cell culture medium and/or glucose is depleted from the second culture medium. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus, wherein between about 100,000 cells/cm² and about 320,000 cells/cm² are inoculated; and (c) harvesting the Enterovirus C virus produced by the cell. In some embodiments, between about 120,000 cells/cm² and about 300,000 cells/cm² are inoculated. In some embodiments, the Enterovirus C virus is a poliovirus serotype selected from the group consisting of S1, S2, and S3. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, the cell is inoculated at a pH that ranges from about 6.8 to about 7.4. In some embodiments, no additional glucose is added to the second cell culture medium and/or glucose is depleted from the second culture medium. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus; and (c) harvesting the Enterovirus C virus produced by the cell, wherein the cell is cultured during steps (a), and/or (b) at a volume/surface ratio of about 0.1 mL/cm² to about 0.3 mL/cm². In some embodiments, the Enterovirus C virus is a poliovirus serotype selected from the group consisting of S1, S2, and S3. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, between about 120,000 cells/cm² and about 300.000 cells/cm² are inoculated. In some embodiments, between about 4,000 cells/cm² and about 16,000 cells/cm² are inoculated. In some embodiments, about 5,000 cells/cm² are inoculated. In some embodiments, step (b) further comprises culturing the inoculated cell under conditions in which the Enterovirus C virus infects the cell and the infected cell produces the Enterovirus C virus, and wherein step (c) comprises lysing the cell to harvest the Enterovirus C virus produced by the cell. In some embodiments, the cell is inoculated at a pH that ranges from about 6.8 to about 7.4. In some embodiments, no additional glucose is added to the second cell culture medium and/or glucose is depleted from the second culture medium. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus, wherein the cell is inoculated at a pH that ranges from about 6.8 to about 7.4; and (c) harvesting the Enterovirus C virus produced by the cell. In some embodiments, the Enterovirus C virus is a poliovirus serotype selected from the group consisting of S1, S2, and S3. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, between about 120,000 cells/cm² and about 300,000 cells/cm² are inoculated. In some embodiments, between about 4,000 cells/cm² and about 16,000 cells/cm² are inoculated. In some embodiments, about 5,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, no additional glucose is added to the second cell culture medium and/or glucose is depleted from the second culture medium. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus, wherein no additional glucose is added to the second cell culture medium and/or glucose is depleted from the second culture medium; and (c) harvesting the Enterovirus C virus produced by the cell. In some embodiments, the Enterovirus C virus is a poliovirus serotype selected from the group consisting of S1, S2, and S3. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, between about 120,000 cells/cm² and about 300,000 cells/cm² are inoculated. In some embodiments, between about 4,000 cells/cm² and about 16.000 cells/cm² are inoculated. In some embodiments, about 5,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, the cell is inoculated at a pH that ranges from about 6.8 to about 7.4. In some embodiments, the method further comprises, after step (c): (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus.

In further aspects, provided herein is a method for producing an Enterovirus C virus, comprising: (a) culturing an adherent cell in a fixed bed comprising a matrix, wherein the cell is cultured in a first cell culture medium; (b) inoculating the cell with the Enterovirus C virus in a second cell culture medium under conditions in which the Enterovirus C virus infects the cell and the infected cell produces the Enterovirus C virus; (c) harvesting the Enterovirus C virus produced by the cell; (d) passing the harvested Enterovirus C virus produced by the cell through a depth filter to produce a first eluate, wherein the first eluate comprises the Enterovirus C virus; (e) binding the first eluate to a cation exchange membrane to produce a first bound fraction, wherein the first bound fraction comprises the Enterovirus C virus; (f) eluting the first bound fraction from the cation exchange membrane to produce a second eluate, wherein the second eluate comprises the Enterovirus C virus; (g) binding the second eluate to an anion exchange membrane to produce a second bound fraction, wherein the second bound fraction comprises the Enterovirus C virus; and (h) eluting the second bound fraction from the anion exchange membrane to produce a purified Enterovirus C virus. In some embodiments, the Enterovirus C virus is a poliovirus serotype selected from the group consisting of S1, S2, and S3.

In some embodiments of any of the above embodiments, the depth filter has a pore size of between about 0.2 μm and about 3 μm. In some embodiments of any of the above embodiments, before step (e) the pH of the first eluate is adjusted to a pH value of about 5.7. In some embodiments of any of the above embodiments, the Enterovirus C virus is a poliovirus serotype selected from the group consisting of S1 and S2. In some embodiments of any of the above embodiments, before step (e) the pH of the first eluate is adjusted to a pH value of about 5.0, and wherein the Enterovirus C virus is poliovirus S3. In some embodiments of any of the above embodiments, a citrate buffer or a phosphate buffer is used to bind the first eluate to the cation exchange membrane. In some embodiments of any of the above embodiments, a buffer comprising polysorbate is used to bind the first eluate to the cation exchange membrane. In some embodiments of any of the above embodiments, the first eluate is bound to the cation exchange membrane at a pH that ranges from about 4.5 to about 6.0. In some embodiments of any of the above embodiments, the first eluate is bound to the cation exchange membrane at between about 8 mS/cm and about 10 mS/cm. In some embodiments of any of the above embodiments, the first bound fraction is eluted by adjusting the pH to about 8.0. In some embodiments of any of the above embodiments, the first bound fraction is eluted by adding from about 0.20 M to about 0.30 M sodium chloride. In some embodiments of any of the above embodiments, the first bound fraction is eluted at between about 20 mS/cm and about 25 mS/cm. In some embodiments of any of the above embodiments, before step (g) the pH of the second eluate is adjusted to a pH value of about 8.0 to about 8.5. In some embodiments of any of the above embodiments, the Enterovirus C virus is a poliovirus serotype selected from the group consisting of S1, S2, and S3, and wherein before step (g) the pH of the second eluate is adjusted to a pH value of about 8.0 to about 8.5. In some embodiments of any of the above embodiments, the Enterovirus C virus is a poliovirus serotype selected from the group consisting of S1 and S3, and before step (g) the pH of the second eluate is adjusted to a pH value of about 8.5. In some embodiments of any of the above embodiments, the Enterovirus C virus is poliovirus S2, and before step (g) the pH of the second eluate is adjusted to a pH value of about 8.0. In some embodiments of any of the above embodiments, a phosphate buffer is used to bind the second eluate to the anion exchange membrane. In some embodiments of any of the above embodiments, a buffer comprising polysorbate is used to bind the second eluate to the anion exchange membrane. In some embodiments of any of the above embodiments, the second eluate is bound to the anion exchange membrane at a pH that ranges from about 7.5 to about 8.5. In some embodiments of any of the above embodiments, the second eluate is bound to the anion exchange membrane at about 3 mS/cm. In some embodiments of any of the above embodiments, the second bound fraction is eluted by adding from about 0.05 M to about 0.10 M sodium chloride. In some embodiments of any of the above embodiments, the second bound fraction is eluted at between about 5 mS/cm and about 10 mS/cm. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, between about 120,000 cells/cm² and about 300,000 cells/cm² are inoculated. In some embodiments, between about 4,000 cells/cm² and about 16,000 cells/cm² are inoculated. In some embodiments, about 5,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, the cell is inoculated at a pH that ranges from about 6.8 to about 7.4. In some embodiments, no additional glucose is added to the second cell culture medium and/or glucose is depleted from the second culture medium.

In some embodiments of any of the above embodiments, the cell is a mammalian cell. In some embodiments of any of the above embodiments, the cell is a Vero cell. In some embodiments of any of the above embodiments, the Vero cell line is selected from the group consisting of WHO Vero 10-87, ATCC CCL-81, Vero 76 (ATCC Accession No. CRL-1587), and Vero C1008 (ATCC Accession No. CRL-1586. In some embodiments of any of the above embodiments, between about 4,000 cells/cm² and about 16,000 cells/cm² are cultured in step (a). In some embodiments of any of the above embodiments, about 5,000 cells/cm² are cultured in step (a). In some embodiments of any of the above embodiments, the first cell culture medium and the second cell culture medium are different. In some embodiments of any of the above embodiments, the method further comprises, between steps (a) and (b), removing the first cell culture medium and rinsing the cell with the second culture medium. In some embodiments of any of the above embodiments, the second cell culture medium is a serum-free medium. In some embodiments of any of the above embodiments, the lactate concentration in the first cell culture medium during step (a) does not exceed about 25 mM. In some embodiments of any of the above embodiments, the lactate concentration in the second cell culture medium during step (b) does not exceed about 15 mM. In some embodiments of any of the above embodiments, the density of oxygen (DO) in the first cell culture medium during step (a) is maintained above about 50%. In some embodiments of any of the above embodiments, density of oxygen (DO) in the second cell culture medium during step (b) is maintained above about 50%. In some embodiments of any of the above embodiments, the density of oxygen (DO) in the first cell culture medium during step (a) is maintained above about 60%. In some embodiments of any of the above embodiments, density of oxygen (DO) in the second cell culture medium during step (b) is maintained above about 60%. In some embodiments of any of the above embodiments, the fixed bed has a bed height of about 2 cm. In some embodiments of any of the above embodiments, the fixed bed has a bed height of about 10 cm. In some embodiments of any of the above embodiments, the matrix is a fiber matrix. In some embodiments of any of the above embodiments, the fiber matrix is a carbon matrix. In some embodiments of any of the above embodiments, the fiber matrix has a porosity between about 60% and 99%. In some embodiments of any of the above embodiments, the porosity is between about 80% and about 90%. In some embodiments of any of the above embodiments, the fiber matrix has a surface area accessible to the cell of between about 150 cm²/cm³ and about 1000 cm²/cm³. In some embodiments of any of the above embodiments, the fiber matrix has a surface area accessible to the cell of between about 10 cm²/cm³ and about 150 cm²/cm³. In some embodiments, the fiber matrix has a surface area accessible to the cell of about 120 cm²/cm³. In some embodiments of any of the above embodiments, at least 5.0×10⁷ TCID50/mL of the Enterovirus C virus is harvested in step (d). In some embodiments of any of the above embodiments, the method further comprises inactivating the Enterovirus C with one or more of beta-propiolactone (BPL), formalin, or binary ethylenimine (BEI). In some embodiments of any of the above embodiments, the Enterovirus C virus is a poliovirus strain selected from the group consisting of LSc,2ab; P712,Ch,2ab; Leon, 12_{a1b}; and any combination thereof.

In further aspects, provided herein is an Enterovirus C virus produced by the method of any of the above embodiments. In some embodiments, the virus comprises one or more antigens. In some embodiments, the virus has been inactivated with one or more of beta-propiolactone (BPL), formalin, or binary ethylenimine (BED).

In further aspects, provided herein is a composition comprising the virus of any of the above embodiments. In further aspects, provided herein is an immunogenic composition comprising the virus of any of the above embodiments. In further aspects, provided herein is a vaccine comprising the virus of any of the above embodiments.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exemplary scheme of upstream processing steps for virus production using a fixed-bed bioreactor in accordance with some embodiments. WVS: working virus seed.

FIGS. 2A & 2B show the impact of cell density at infection (CDI) on cell productivity, as reflected by poliovirus D-antigen production. Productivity is plotted in two ways: “volumetric” productivity (DU/mL; FIG. 2A) and “per cell” productivity (DU/10⁶ cells; FIG. 2B).

FIG. 3 shows the impact of virus multiplicity of infection (MOI) on cell productivity over time, as reflected by volumetric poliovirus D-antigen production (DU/mL).

FIG. 4 shows the impact of cell growth in an iCELLis NANO® (diamonds) compared to T-flask (CS control, squares) on virus stability.

FIGS. 5A-5C show the impacts of pH and dissolved oxygen (DO) regulation on cell productivity in an iCELLis NANO® (FIG. 5A), as well as the pH (FIG. 5B) and DO levels (FIG. 5C) over time with regulation (“Control”) and without regulation (“No regulation”).

FIG. 6 shows the volumetric production of extracellular (squares) and intracellular (diamonds) D-antigen over time in an iCELLis NANO®.

FIG. 7A compares per cell productivity, glucose shortage, and lactate dehydrogenase (LDH) activity at infection for cells grown on microcarriers (e.g., CYTODEX™) as compared to two batches grown in an iCELLis NANO® (“NANO 1” and “NANO 2”). FIG. 7B compares per cell productivity of cells grown on microcarriers as described herein (e.g., CYTODEX™), cells grown on microcarriers as described in literature (“Lit. Cytodex™”), and two batches grown in an iCELLis NANO® (“Cytodex copy-paste in iCELLis” and “Best run”). FIGS. 7C & 7D compare per cell productivity of cells grown on microcarriers as described herein or cells grown on microcarriers as described in literature (“Cytodex™ T” and “Cytodex™ lit” in FIG. 7C) with two batches grown in an iCELLis NANO® (“H” and “P” in FIG. 7D). FIGS. 7E & 7F show the effect of initial glucose concentration in cell culture medium at infection phase on cell productivity (D-antigen/cm²). FIG. 7G shows the effect of glucose shortage prior to infection on volumetric cell productivity (D-antigen/mL) over time. FIG. 7H shows the effect of adding extra glucose at infection on volumetric cell productivity (D-antigen/mL) over time.

FIGS. 8A-8C show increased viral yields due to addition of polysorbate (Tween-80).

FIGS. 9A & 9B illustrate an exemplary scheme of downstream processing steps for virus production using a fixed-bed bioreactor in accordance with some embodiments. A comparison between the improved downstream process described herein and an existing process is shown in FIG. 9A. A detailed flow diagram of downstream purification and inactivation is shown in FIG. 9B.

FIG. 10 provides a summary of three sets of experimental conditions (Experiments D, 8, and C) used to assess effect of modifying specific downstream processing parameters.

FIGS. 11A-11E show the purification of poliovirus produced by the experimental conditions shown in FIG. 10 using SDS-PAGE silver staining. Shown are the purification of S2 virus from Experiment 8 (FIG. 11A), purification of S2 virus from Experiment D (FIG. 11B), comparison of S2 virus purified by Experiment D (lane 1 in FIG. 11C) vs. that purified using existing protocol (lane 2 in FIG. 11C), purification of S3 virus from Experiment D (FIG. 11D), comparison of S3 virus purified by Experiment D (lane 1 in FIG. 11E) vs. that purified using existing protocol (lane 2 in FIG. 11E). Each lane represents the product of a specific purification step, as indicated. FT: flow-through.

FIGS. 12A & 12B show the effect of various combinations of buffer compositions and pH on D-antigen (DU) elution using anion exchange chromatography. 5× (FIG. 12A) and 3× (FIG. 12B) dilutions are shown. Conditions with (+) and without (−) polysorbate (“Tween”) are as indicated.

FIGS. 13A & 13B show the effect of various combinations of buffer compositions and pH on D-antigen (DU) elution using cation exchange chromatography. 5× (FIG. 13A) and 3× (FIG. 13B) dilutions are shown. Conditions with (+) and without (−) polysorbate (“Tween”) are as indicated.

FIGS. 14A-14D show binding efficiency as a function of pH using cation/anion exchange chromatography and elution with citrate (diamonds), Tris (squares), or phosphate (triangles) buffer. FIG. 14A: anion exchange without polysorbate. FIG. 14B: anion exchange with polysorbate. FIG. 14C: cation exchange without polysorbate. FIG. 14D: cation exchange with polysorbate. All conditions use 5× dilution factor. FIGS. 14E-14H show anion exchange chromatography elution of virus at AcroDisc® scale. FIGS. 14E & 14G show elution profile obtained using 4× dilution factor and Tris pH 8.0 buffer without and with polysorbate, respectively. FIG. 14F shows virus yield of each fraction of the experiment shown in FIG. 14E. FIG. 14H shows the purity of each fraction of the experiment shown in FIG. 14G using SDS-PAGE silver staining. FIGS. 14I-14L show cation exchange chromatography elution of virus at AcroDisc® scale. FIGS. 14I & 14K show elution profile obtained using 4× dilution factor and citrate pH 5.5 buffer without and with polysorbate, respectively. FIG. 14J shows virus yield of each fraction of the experiment shown in FIG. 14I. FIG. 14I, shows the purity of each fraction of the experiment shown in FIG. 14K using SDS-PAGE silver staining.

FIGS. 15A-15C show the experimental setup for testing the effect of cation and anion exchange conditions on recovery. FIG. 15A: cation exchange conditions tested. FIG. 15B: anion exchange conditions tested. FIG. 15C: effect of each condition on step and overall recovery. 0.05% TWEEN®-80 was present in all buffers.

FIGS. 16A & 16B show the experimental setup for testing the effect of increasing buffer concentration and lowering cation exchange elution pH and anion exchange loading pH. FIG. 16A: conditions tested. FIG. 16B: results of increasing buffer concentration and lowering cation exchange elution pH and anion exchange loading pH on recovery (results of DSP1.0 are given in upper two rows, while results of DSP1.1 are given in lower two rows).

FIG. 17A shows the effect of dilution strength on recovery (as assayed by total D-antigen as a percentage of total load). Recovery is depicted in the elution as well as the flow-through and wash (“FT+Wash”). Please note that no DU was recovered in the flow-through even under no dilution conditions. FIG. 17B shows the percentage of S2 poliovirus (e.g., as assayed by D-antigen) in the flow-through as a function of dilution factor used to load the cation exchange membrane.

FIGS. 18A & 18B show the effects on changing elution buffer on anion exchange elution profiles. Depicted are the elution profiles obtained as a result of using a pH-based elution with pH 8.0 phosphate buffer (FIG. 18A) and using a salt-based elution with NaCl in pH 8.0 phosphate buffer (FIG. 18B).

FIG. 19 provides a schematic diagram of an exemplary downstream processing flow for scaling up virus production.

FIGS. 20A & 20B show diagrams summarizing two full production processes in accordance with some embodiments.

FIG. 21A provides a full process flow chart for virus production using an iCELLis® 500/66 m² system. FIG. 21B shows optimized parameters for upstream process steps using an iCELLis® 500/66 m² system.

FIG. 22 shows the results of a full 25 L scale production process.

FIG. 23A shows downstream processing steps for a full 25 L scale production process. FIGS. 23B-23D show elution of virus from cation exchange chromatography at two different conductivities.

FIGS. 23E-23G show the elution profiles from anion exchange chromatography from the full 25 L scale production process. FIG. 23H shows downstream process parameters for purification of S2 virus. FIG. 23I shows the volume; D-antigen titer, total amount, and recovery; total protein; and protein/D-antigen ratio for each step of the downstream process.

FIGS. 24A-24C show the differences in upstream process parameters of virus production in iCELLis® 500/66 m² and NANO systems and their effect on overall productivity (DU).

FIG. 25A summarizes upstream processing steps for viral harvest and purification. FIG. 25B summarizes downstream processing steps for viral harvest and purification.

FIG. 26A shows pH loading of strain S1 onto the cation exchange membrane. FIGS. 26B & 26C show NaCl elution of strain S1 from the cation exchange membrane.

FIG. 27A shows pH loading of strain S1 onto the anion exchange membrane. FIG. 27B shows NaCl elution of strain S1 from the anion exchange membrane.

FIG. 28A shows pH loading of strain S3 onto the cation exchange membrane. FIGS. 28B & 28C show NaCl elution of strain S3 from the cation exchange membrane.

FIG. 29A shows pH loading of strain S3 onto the anion exchange membrane. FIG. 29B shows NaCl elution of strain S3 from the anion exchange membrane.

FIGS. 30A & 30B show downstream process steps using NaCl (FIG. 30A) and pH (FIG. 30B) elutions, respectively, in accordance with some embodiments.

FIGS. 31A & 31B show viral recovery at each process step from a full process run using NaCl (FIG. 31A) and pH (FIG. 31B) elutions, respectively, in accordance with some embodiments. Strain S2 was used for these experiments.

FIG. 32 shows the chromatograph obtained from NaCl elution of anion exchange membrane using process run with strain S2. Particular fractions and their corresponding volumes are as indicated.

FIGS. 33A & 33B show VP1, VP2, and VP3 obtained from process run using strain S2 after various process steps. FIG. 33A shows results of NaCl elution, and FIG. 33B shows results of pH elution.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Preshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Erperimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Maual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Cell Culture

Certain aspects of the present disclosure relate to methods for producing an Enterovirus C virus (e.g., poliovirus S1, S2, or S3). Producing an Enterovirus C may be useful, e.g., for vaccines and/or immunogenic compositions including, without limitation, purified viruses, inactivated viruses, attenuated viruses, recombinant viruses, or purified and/or recombinant viral proteins for subunit vaccines.

In some embodiments, the cell of the present disclosure is a mammalian cell (e.g., a mammalian cell line). Cell lines suitable for growth of the at least one virus of the present disclosure are preferably of mammalian origin, and include but are not limited to: Vero cells (from monkey kidneys), horse, cow (e.g. MDBK cells), sheep, dog (e.g. MDCK cells from dog kidneys, ATCC CCL34 MDCK (NBL2) or MDCK 33016, deposit number DSM ACC 2219 as described in WO97/37001), cat, and rodent (e.g. hamster cells such as BHK21-F, HKCC cells, or Chinese hamster ovary cells (CHO cells)), and may be obtained from a wide variety of developmental stages, including for example, adult, neonatal, fetal, and embryo. In certain embodiments, the cells are immortalized (e.g. PERC.6 cells, as described in WO01/38362 and WO02/40665, and as deposited under ECACC deposit number 96022940). In preferred embodiments, mammalian cells are utilized, and may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g., epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. WO97/37000 and WO97/37001 describe production of animal cells and cell lines that capable of growth in suspension and in serum free media and are useful in the production and replication of viruses.

In some embodiments, the cell is a mammalian kidney cell. Examples of suitable mammalian kidney cells include, without limitation, MDCK, MDBK, BHK-21, Vero, HEK, and HKCC cells.

In certain embodiments, the cell is a Vero cell. Examples of suitable Vero cell lines include, without limitation, WHO Vero 10-87, ATCC CCL-81, Vero 76 (ATCC Accession No. CRL-1587), or Vero C1008 (ATCC Accession No. CRL-1586).

In some embodiments, the cell is an adherent cell. As used herein, an adherent cell may refer to any cell that adheres or anchors to a substrate during culturing.

Culture conditions for the above cell types are known and described in a variety of publications, or alternatively culture medium, supplements, and conditions may be purchased commercially, such as for example, as described in the catalog and additional literature of Cambrex Bioproducts (East Rutherford, N.J.).

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

Certain aspects of the methods of the present disclosure relate to the density at which cells are cultured. In some embodiments, the density or absolute number of cells cultured in a first culture medium may refer to the density or absolute number of cells with which the cell culture is seeded, i.e., before viral inoculation. As described herein, and without wishing to be bound to theory, it is thought that culturing cells at a lower density may be advantageous to reduce the cell density at viral inoculation, prolong the cells' growth phase, and/or reduce factors including without limitation the size of the inoculum, labor time, foot-print in the pre-culture step, number of incubators, and/or the risk of contamination.

In some embodiments, between about 4,000) cells/cm² and about 16,000 cells/cm² are cultured. For example, in some embodiments, the seeding density to start the initial cell culture is between about 4,000 cells/cm² and about 16,000 cells/cm². In some embodiments, about 4,000 cells/cm²; about 5,000 cells/cm²; about 6,000 cells/cm²; about 7,000 cells/cm²; about 8,000 cells/cm²; about 9,000 cells/cm²; about 10,000 cells/cm²; about 11,000 cells/cm²; about 12,000 cells/cm²; about 13,000 cells/cm²; about 14,000 cells/cm²; about 15,000 cells/cm²; or about 16,000 cells/cm² are cultured, including any value therebetween. In some embodiments, the cell density before inoculation is less than about any of the following densities (in cells/cm²): 16,000; 15,500; 15,000; 14,500; 14,000; 13,500; 13,000; 12,500; 12,000; 11,500; 11,000; 10,500; 9,000; 8,500; 8,000; 7,500; 7,000; 6,500; 6,000; 5,500; 5,000; or 4,500. In some embodiments, the cell density before inoculation is greater than about any of the following densities (in cells/cm): 4,000; 4,500; 5,000; 5,500; 6,000; 6,500; 7,000; 7,500; 8,000; 8,500; 9,000; 9,500; 10,000; 10,500; 11,000; 11,500; 12,000; 12,500; 13,000; 13,500; 14,000; 14,500; 15,000; or 15,500. That is, the cell density before inoculation can be any of a range of densities having an upper limit of 16,000; 15,500; 15,000; 14,500; 14,000; 13,500; 13,000; 12,500; 12,000; 11,500; 11,000; 10,500; 9,000; 8,500; 8,000; 7,500; 7,000; 6,500; 6,000; 5,500; 5,000; or 4,500 and an independently selected lower limit of 4,000; 4,500; 5,000; 5,500; 6,000; 6,500; 7,000; 7,500; 8,000; 8,500; 9,000; 9,500; 10,000; 10,500; 11,000; 11,500; 12,000; 12,500; 13,000; 13,500; 14,000; 14,500; 15,000; or 15,500; wherein the lower limit is less than the upper limit. In certain embodiments, about 5,000 cells/cm² are cultured. In some embodiments, between about 4,000 cells/cm² and about 16,000 cells/cm² are used as an initial seeding density, then cultured until reaching between about 120,000 cells/cm² and about 300,000 cells/cm² for inoculation with an Enterovirus C (e.g., poliovirus S1, S2, or S3).

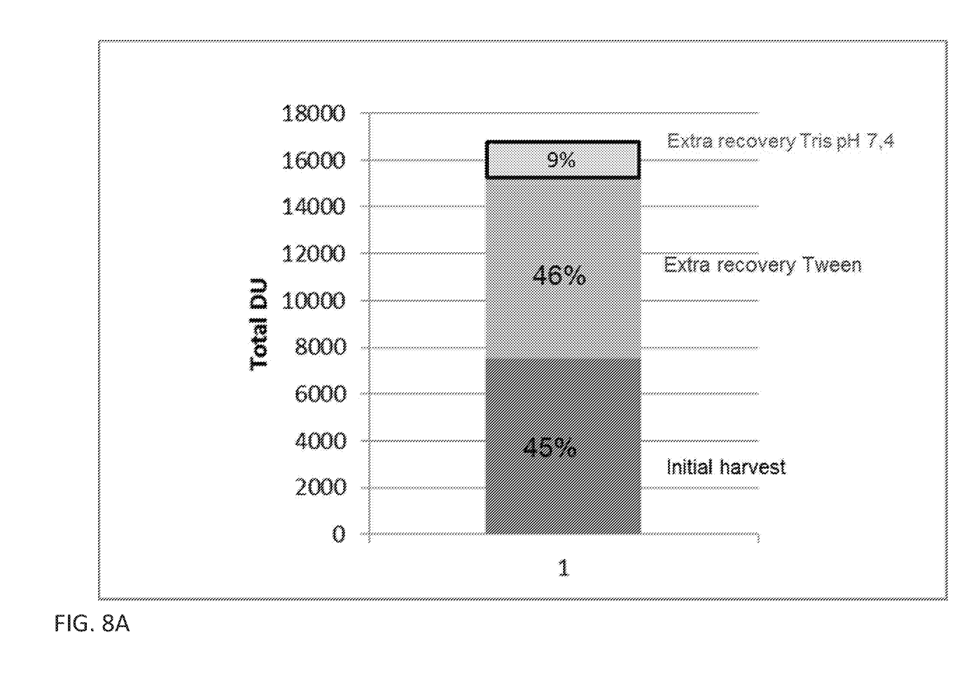

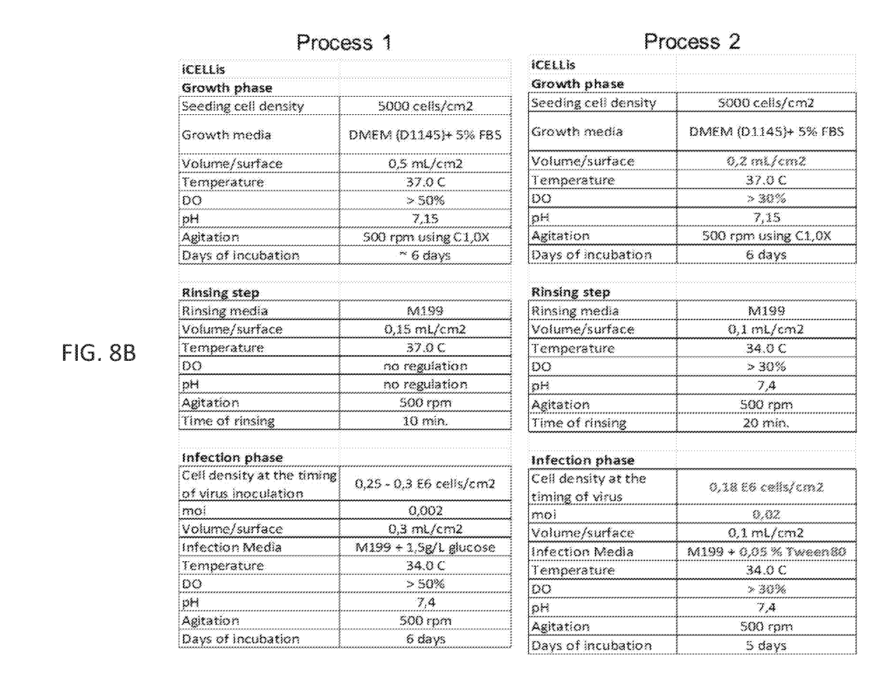

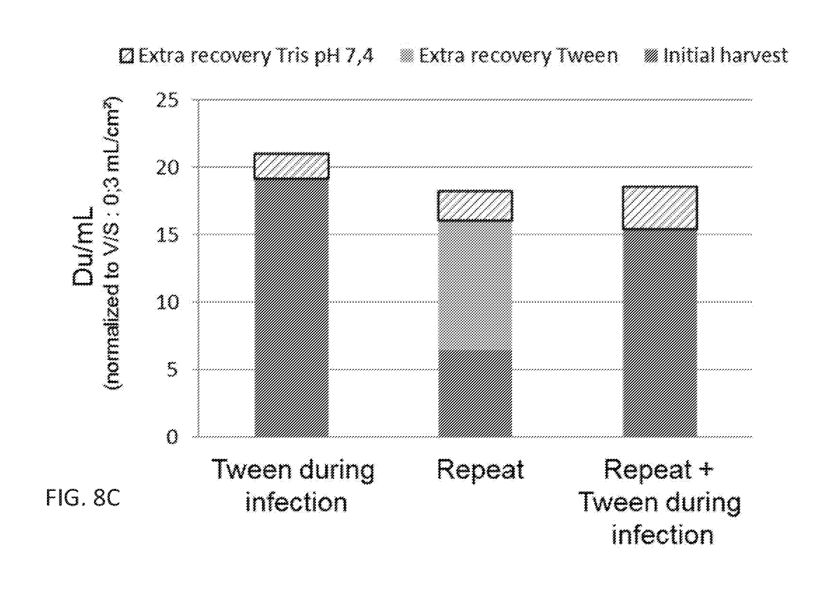

In some embodiments, a cell of the present disclosure is inoculated with an Enterovirus C virus of the present disclosure (e.g., poliovirus S1, S2, or S3) under conditions in which the Enterovirus C virus infects the cell. Conditions under which an Enterovirus C infects a cell are known in the art and may depend on the type of cell, the type of Enterovirus C, the culture medium, temperature, cell density, viral density (e.g., MOI), cell growth rate, number of cell passages, and so forth. Exemplary descriptions of conditions under which an Enterovirus infects a cell are provided infra. For example, in some embodiments, a cell culture may be cultured and/or inoculated using one or more of the conditions described in FIG. 8B in any combination.

Certain aspects of the methods of the present disclosure relate to the cell density at the time of viral inoculation. As described herein, and without wishing to be bound to theory, it is thought that the cell density at infection may impact virus production (e.g., viral productivity). An optimal cell density at viral inoculation may result in increased specific (per cell) productivity, volumetric (per mL of harvest) productivity, and/or stability, as well as reduced media consumption and/or contaminants in the harvest.

In some embodiments, between about 4,000 cells/cm² and about 16,000 cells/cm² are inoculated with Enterovirus C (e.g., poliovirus S1, S2, or S3). In some embodiments, between about 120,000 cells/cm² and about 300,000 cells/cm² are inoculated with Enterovirus C (e.g., poliovirus S1, S2, or S3). In some embodiments, between about 150,000 cells/cm² and about 300,000 cells/cm² are inoculated with Enterovirus C. In some embodiments, between about 120,000 cells/cm² and about 200,000 cells/cm² are inoculated with Enterovirus C. In some embodiments, the cell density at inoculation is less than about any of the following densities (in cells/cm²): 300,000; 275,000; 250,000; 225,000; 200,000; 175,000; 150,000; 125,000; 100,000; 75,000; 50,000; 45,000; 40,000; 35,000; 30,000; 25,000; 20,000, 17,500; 15,000; 12,500; 10,000; 7,500; or 5,000. In some embodiments, the cell density at inoculation is greater than about any of the following densities (in cells/cm²): 2,500; 5,000; 7,500; 10,000; 12,500; 15,000; 17,500; 20,000; 25,000; 30,000; 35,000; 40,000; 45,000; 50,000; 75,000; 100,000; 120,000; 150,000; 175,000; 200,000; 225,000; 250,000; or 275,000. That is, the cell density at inoculation can be any of a range of densities having an upper limit of 300,000; 275,000; 250,000; 225,000; 200,000; 175,000; 150,000; 125,000; 100,000; 75,000; 50,000; 45,000; 40,000; 35,000; 30,000; 25,000; 20,000, 17,500; 15,000; 12,500; 10,000; 7,500; or 5,000 and an independently selected lower limit of 2,500; 5,000; 7,500; 10,000; 12,500; 15,000; 17,500; 20,000; 25,000; 30,000; 35,000; 40,000; 45,000; 50,000; 75,000; 100,000; 120,000; 150,000; 175,000; 200,000; 225,000; 250,000; or 275,000; wherein the lower limit is less than the upper limit. In some embodiments, about 5,000 cells/cm² are inoculated with Enterovirus C (e.g., poliovirus S1, S2, or S3).

Certain aspects of the methods of the present disclosure relate to the volume/surface ratio at which a cell of the present disclosure is cultured (e.g., prior to, during, or after inoculation with an Enterovirus C virus, such as poliovirus S1, S2, or S3). As described herein, and without wishing to be bound to theory, it is thought that the volume/surface ratio at which a cell is cultured may impact virus production (e.g., viral productivity). An optimal volume/surface ratio may result in increased specific (per cell) productivity, volumetric (per mL of harvest) productivity, and/or stability, as well as reduced media consumption and/or contaminants in the harvest.

In some embodiments, the volume/surface ratio at which a cell of the present disclosure is cultured is from about 0.1 mL/cm² to about 0.3 mL/cm². In some embodiments, the volume/surface ratio at which a cell of the present disclosure is cultured refers to the conditions under which the cell is cultured prior to inoculation. In some embodiments, the volume/surface ratio at which a cell of the present disclosure is cultured refers to the conditions under which the cell is cultured during inoculation. In some embodiments, the volume/surface ratio at which a cell of the present disclosure is cultured refers to the conditions under which the cell is cultured after inoculation. In some embodiments, the cell is an adherent cell, and the cell is cultured in a fixed bed with a matrix, e.g., as described herein and/or otherwise known in the art. In some embodiments, the volume/surface ratio is less than about any of the following ratios (in mL/cm²): 0.3, 0.275, 0.25, 0.225, 0.2, 0.175, 0.15, or 0.125. In some embodiments, the volume/surface ratio is greater than about any of the following ratios (in mL/cm²): 0.1, 0.125, 0.150, 0.175, 0.2, 0.225, 0.25, or 0.275. That is, the volume/surface ratio can be any of a range of ratios having an upper limit of 0.3, 0.275, 0.25, 0.225, 0.2, 0.175, 0.15, or 0.125 and an independently selected lower limit of 0.1, 0.125, 0.150, 0.175, 0.2, 0.225, 0.25, or 0.275; wherein the lower limit is less than the upper limit.

Certain aspects of the methods of the present disclosure relate to the MOI of enterovirus used to inoculate a cell culture of the present disclosure. MOI is used herein consistent with its accepted meaning in the art, i.e., a known or predicted ratio of viral agent (e.g., Enterovirus C virus) to viral target (e.g., a cell of the present disclosure). MOI is known in the art to affect the percentage of cells in a culture that are infected with at least one virus. While a higher MOI may increase viral productivity, at a certain range of MOI infection rate may saturate, and reducing the MOI after reaching a threshold or desired infection rate may lower the volume and costs of the corresponding virus seed bank.

In some embodiments, the cell is inoculated with the Enterovirus C virus (e.g., poliovirus S1, S2, or S3) at an MOI of between about 0.01 and about 0.0009. In some embodiments, the MOI is less than about any of the following MOIs: 0.010, 0.008, 0.005, 0.002, or 0.0010. In some embodiments, the MOI is greater than about any of the following MOIs: 0.0009, 0.0010, 0.002, 0.005, 0.008, or 0.010. That is, the MOI can be any of a range of MOIs having an upper limit of 0.010, 0.008, 0.005, 0.002, or 0.0010 and an independently selected lower limit of 0.0009, 0.0010, 0.002, 0.005, 0.008, or 0.010, wherein the lower limit is less than the upper limit.

Certain aspects of the methods of the present disclosure relate to the pH in which a cell of the present disclosure is inoculated (e.g., a pH of the cell culture medium containing the cell). As described herein, pH can affect cellular virus production.

In some embodiments, the cell is inoculated with the Enterovirus C virus (e.g., poliovirus S1, S2, or S3) at a pH that ranges from about 6.8 to about 7.4. In some embodiments, the pH at inoculation is less than about any of the following pHs: 7.4, 7.3, 7.2, 7.1, 7.0, or 6.9. In some embodiments, the pH at inoculation is greater than about any of the following pHs: 6.8, 6.9, 7.0, 7.1, 7.2, or 7.3. That is, the pH at inoculation can be any of a range of pHs having an upper limit of 7.4, 7.3, 7.2, 7.1, 7.0, or 6.9 and an independently selected lower limit of 6.8, 6.9, 7.0, 7.1, 7.2, or 7.3, wherein the lower limit is less than the upper limit. For example, in some embodiments, the pH at inoculation may be about 6.8, about 6.9, about 7.0, about 7.1, about 7.2, about 7.3, or about 7.4.

Upon infection, an infected cell of the present disclosure may be cultured (e.g., in a fixed bed of the present disclosure) in a second cell culture medium. A cell cultured as described herein may be cultured in a first culture medium before viral inoculation and a second culture medium at and/or after viral inoculation. For example, in some embodiments, a cell of the present disclosure may be cultured in a first cell culture medium, then the first cell culture medium may be removed, the cell may optionally be rinsed (e.g., with an aqueous buffered solution such as PBS), and a second culture medium may be added to the cell. In some embodiments, the second culture medium may contain the Enterovirus C virus (e.g., poliovirus S1, S2, or S3) for inoculation of the cell. In some embodiments, the first culture medium and the second culture medium are the same culture medium (e.g., having the same composition, which in some embodiments is not necessarily the same physical medium). In other embodiments, the first culture medium and the second culture medium are different (e.g., having a different composition).

In some embodiments, the first cell culture medium contains serum (e.g., fetal bovine serum). Any type of serum suitable for growth of the cultured cell may be used. Examples of sera in the art include without limitation fetal bovine serum, fetal calf serum, horse serum, goat serum, rabbit serum, rat serum, mouse serum, and human serum. In some embodiments, the first cell culture medium contains less than 10% serum (e.g., fetal bovine serum), and the cell is not adapted to serum free medium. In certain embodiments, the first cell culture medium contains 5% serum (e.g., fetal bovine serum).

In some embodiments, the second culture medium is a serum-free medium. In some embodiments, the second culture medium is a protein free medium. A medium (e.g., a culture medium of the present disclosure) is referred to as a serum-free medium in the context of the present disclosure in which there are no additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

In some embodiments, surfactant and/or dextran is added to the second cell culture medium, e.g., during inoculation of the cell with the Enterovirus C virus (e.g., poliovirus S1, S2, or S3) or from approximately one hour to approximately four hours prior to harvest. A variety of surfactants may be suitably used in a cell culture medium of the present disclosure, including without limitation polysorbates such as polysorbate 20 (also known as TWEEN® 20), 40, 60, and 80 (also known as TWEEN® 80); and polyethylene glycol-based surfactants such as the TRITON™ series (e.g., TRITON™ X-100, Dow Chemical) and IGEPAL® CA-630 (Rhodia Operations)/NONIDET™ P-40 (Shell Chemical). In some embodiments, the amount of surfactant and/or dextran added to the cell culture medium ranges from 0.005% to 0.05% (e.g., as a v/v percentage of the volume of cell culture medium), such as 0.005%, 0.010%, 0.015%, 0.020%, 0.025%, 0.030%, 0.035%, 0.040%, 0.045%, or 0.050%, including any values therebetween. In certain embodiments, 0.05% polysorbate is added. In certain embodiments, 0.005% polysorbate is added.

As described herein, addition of surfactant and/or dextran to a cell culture medium during/at or prior to inoculation with virus was found to promote a significant increase in total viral harvest and productivity. In some embodiments, the yield of harvested Enterovirus C virus is increased by the addition of a surfactant (e.g., during or prior to harvest) and/or dextran as compared to a yield of Enterovirus C virus harvested in the absence of the surfactant and/or dextran. For example, in some embodiments, the yield of harvested Enterovirus C virus is increased by the addition of a surfactant (e.g., during or prior to harvest) and/or dextran by at least about 10%, at least about 20%, at least about 30%, at least about 40%, at least about 50%, at least about 60%, at least about 70%, at least about 80%, at least about 90%, at least about 100%, at least about two-fold, at least about three-fold, at least about four-fold, at least about five-fold, at least about six-fold, at least about seven-fold, at least about eight-fold, at least about nine-fold, or at least about ten-fold, as compared to a yield of Enterovirus C virus harvested in the absence of the surfactant and/or dextran. In certain embodiments, the yield of harvested Enterovirus C virus is increased by the addition of a surfactant (e.g., during or prior to harvest) and/or dextran by about 10%, about 20%, about 30%, about 40%, about 50%, about 60%, about 70%, about 80%, about 90%, about 100%, about two-fold, about three-fold, about four-fold, about five-fold, about six-fold, about seven-fold, about eight-fold, about nine-fold, or about ten-fold, as compared to a yield of Enterovirus C virus harvested in the absence of the surfactant and/or dextran.

In some embodiments, the cell culture is in a fixed bed bioreactor with a matrix (e.g., using adherent cells). In some embodiments, the cell culture is a liquid culture (e.g., using cells adapted for growth in suspension). In some embodiments, the cells are cultured on microcarriers. In some embodiments, the cell culture is in a bioreactor. A variety of bioreactors suitable for growth in suspension and using adherent cells are known in the art and/or described herein.

In some embodiments, polysorbate is added to the second cell culture medium during inoculation with the virus. In other embodiments, polysorbate is added to the second cell culture medium from approximately one hour to approximately four hours prior to harvest. In some embodiments, the polysorbate is added to the second cell culture after approximately two, three, or four hours prior to harvest. In some embodiments, the polysorbate is added to the second cell culture up to approximately one, two, or three hours prior to harvest. That is, polysorbate may be added to the second cell culture at any time prior to harvest having an upper limit of approximately two, three, or four hours and an independently selected lower limit of approximately one, two, or three hours, wherein the lower limit is less than the upper limit. For example, in some embodiments, polysorbate is added to the second cell culture approximately one, approximately two, approximately three, or approximately four hours prior to harvest.

Certain aspects of the methods of the present disclosure relate to the amount of glucose present in a cell culture medium during, or immediately prior to, inoculation of a cell with an Enterovirus C virus of the present disclosure (e.g., poliovirus S1, S2, or S3). As described herein, metabolic stress (e.g., as evidenced by low available glucose levels in a cell culture medium and/or lactate dehydrogenase activity in cultured cells) may enhance cellular production of virus. In some embodiments, glucose is depleted from a second cell culture medium of the present disclosure. For example, the cell culture medium may be replaced with a cell culture medium having a lower (e.g., depleted) level of glucose, or cells may be grown in the second cell culture medium such that glucose in the cell culture medium is depleted by cellular utilization without replacement/supplementation with exogenous glucose. In some embodiments, no additional glucose is added to a second cell culture medium of the present disclosure. In some embodiments, cells cultured in a cell culture medium of the present disclosure experience glucose shortage (e.g., a reduced amount of glucose relative to a starting amount present in the cell culture) at least 24 hours, at least 48 hours, or at least 72 hours prior to inoculation. In some embodiments, a cell culture glucose level below 250 mg/L indicates glucose shortage on the following day. In some embodiments, cells cultured in a cell culture medium of the present disclosure show increased lactate dehydrogenase (LDH) activity and/or increased lactate production at, or immediately prior to, infection, as compared to cells cultured through standard methods.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, bimaviruses, circoviruses, and/or parvoviruses [WO2006/027698].

Other parameters of cell culturing and the cell culture medium may also affect virus production. In some embodiments, a cell of the present disclosure may be cultured in a first and/or a second culture medium of the present disclosure at a desired volume/surface ratio. Without wishing to be bound to theory, the volume/surface ratio at which a cell is cultured may affect parameters such as cell productivity, cell size, growth rate, and/or access to nutrients and other components in the culture medium. In certain embodiments, the cell is cultured (e.g., before, during, and/or after viral inoculation) at a volume/surface ratio of about 0.3 mL/cm².

Under some conditions, a cell of the present disclosure may produce lactate during culturing. It is known in the art that cells in culture may produce lactate as a result of glycolytic or anaerobic-type metabolism. Without wishing to be bound to theory, it is thought that excessive lactate in cell culture medium may negatively impact cell growth, metabolism, and/or virus productivity, e.g., by reducing the pH of the culture medium. Further, excessive lactate production by cultured cells may indicate a cellular metabolic state that may not be desired for virus production and/or growth. In some embodiments, the lactate concentration in the first cell culture medium and/or the second cell culture medium does not exceed about 25 mM. Methods for measuring lactate concentration in a cell culture medium are known in the art and include without limitation use of a lactometer, a lactate enzymatic assay (e.g., through using lactate dehydrogenase and a colorimetric or fluorometric detection reagent), a microdialysis sampling device, and the like.

In some embodiments, an infected cell of the present disclosure may be cultured (e.g., in a fixed bed of the present disclosure) in a second cell culture medium under which the infected cell produces the Enterovirus C virus (e.g., poliovirus S1, S2, or S3). Conditions under which an infected cell produces an Enterovirus C are known in the art and may depend on the type of cell, the type of Enterovirus C, the culture medium, temperature, cell density, viral density (e.g., MOI), cell growth rate, number of cell passages, and so forth. Exemplary descriptions of conditions under which an Enterovirus infects a cell are provided infra.

Other aspects of the methods described herein relate to the density of oxygen (DO) in a cell culture medium. Maintaining suitable oxygen levels in a cell culture medium may promote cell growth and/or virus productivity by providing oxygen for cellular respiration. In addition, oxygen utilization by cells in a cell culture may reflect their state of health, growth, and/or metabolism. In some embodiments, the density of oxygen in the first cell culture medium and/or the second cell culture medium is maintained above about 50%. Methods for maintaining and/or measuring DO in a cell culture medium are known in the art and may include without limitation automatic oxygen injection, exposing the cell culture medium to oxygen (preferably while minimizing the risk of contamination), using an oxygen controller, using an oxygen sensor, and so forth. In some embodiments, the density of oxygen (DO) in a first cell culture medium of the present disclosure is maintained above about 50%. In some embodiments, the density of oxygen (DO) in a second cell culture medium of the present disclosure is maintained above about 50%. In some embodiments, the density of oxygen (DO) in a first cell culture medium of the present disclosure is maintained above about 60%. In some embodiments, the density of oxygen (DO) in a second cell culture medium of the present disclosure is maintained above about 60%.

In some embodiments, a first cell culture medium of the present disclosure contains a lactate concentration that does not exceed about 25 mM. In some embodiments, a second cell culture medium of the present disclosure contains a lactate concentration that does not exceed about 25 mM. Methods for measuring lactate concentration in a cell culture medium are known in the art, e.g., as described herein.

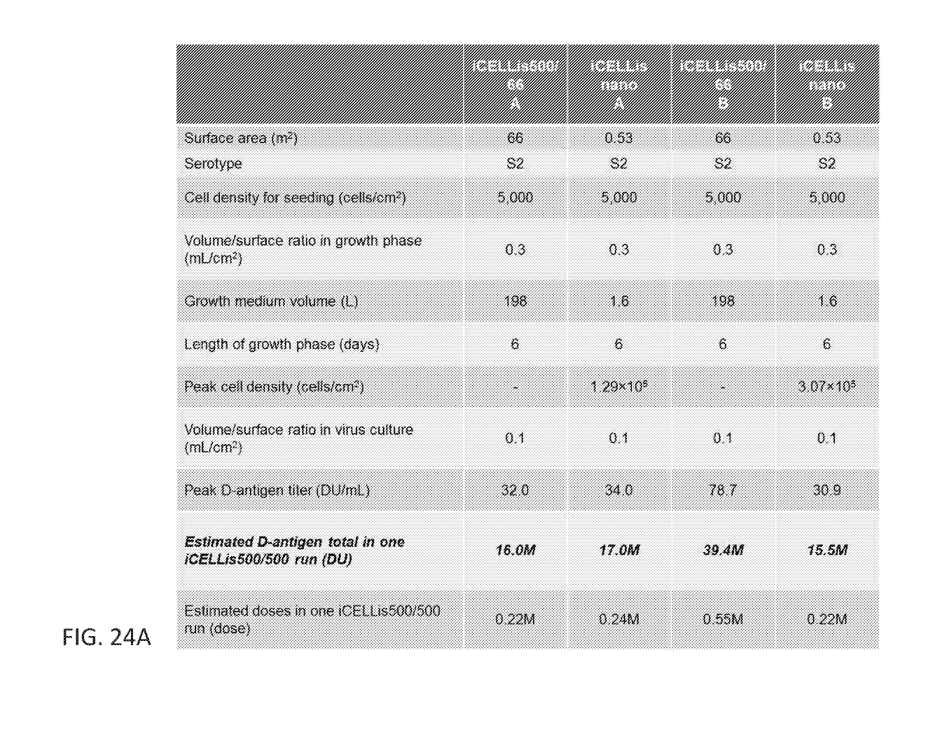

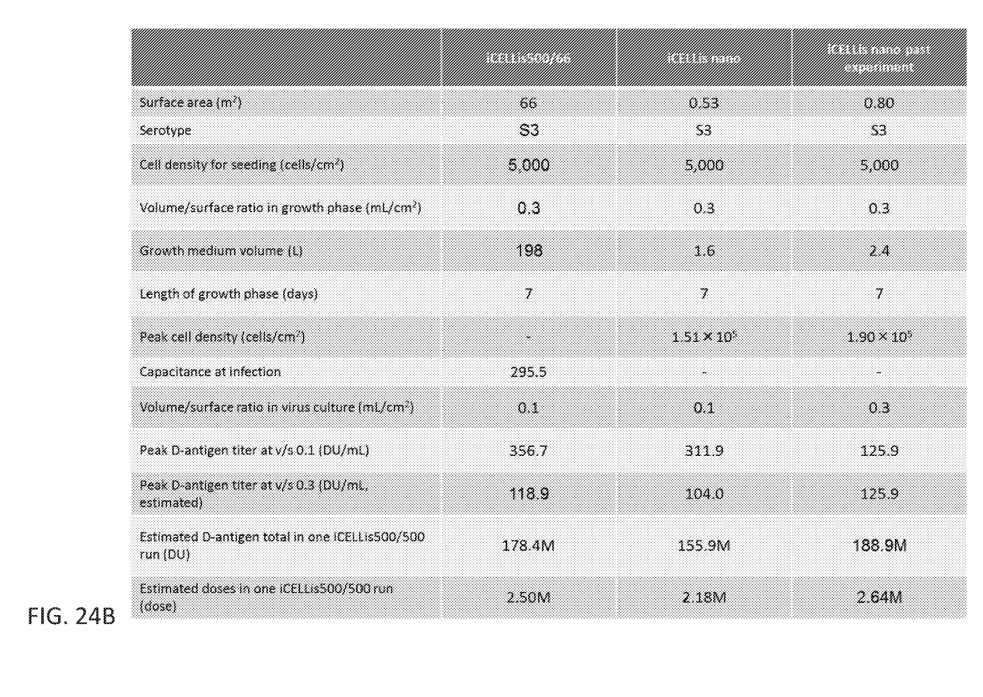

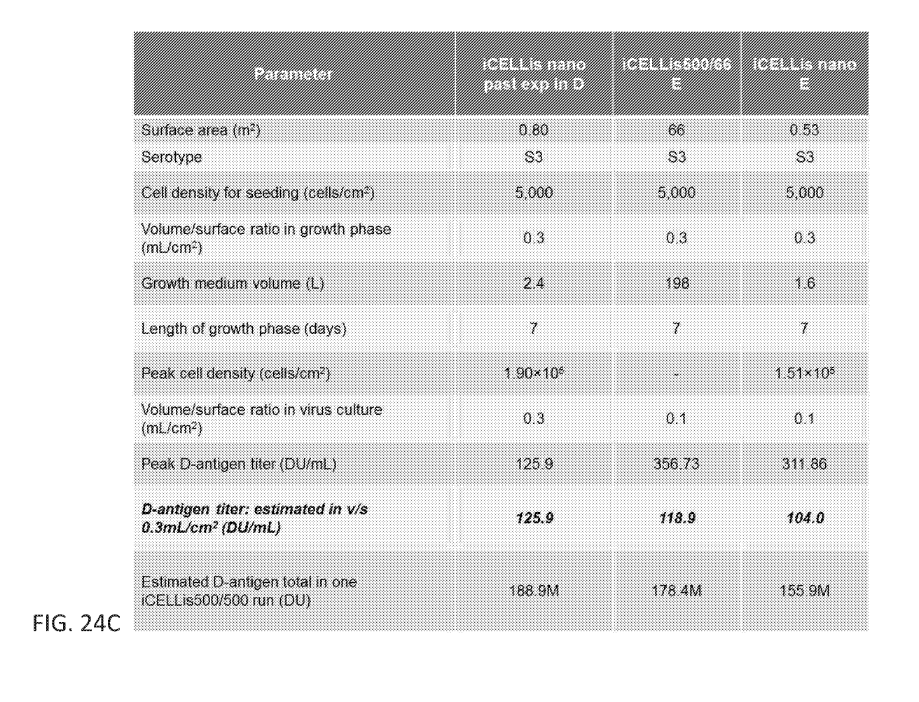

Exemplary culture parameters and conditions are described herein. It is contemplated that cell culturing techniques and parameters as described above may employ one or more of the conditions described in Example 11, and/or in reference to FIGS. 24A-24C, in any combination.

Various methods known in the art may be used to measure the titer of infectious virus produced by the methods of the present disclosure. Such methods include without limitation an endpoint dilution assay (e.g., to determine a TCID50 value), a protein assay, quantitative transmission electron microscopy (TEM), a plaque assay, a focus forming assay (FFA), and so forth. In some embodiments, virus titer is measured in TCID50/mL. In some embodiments, at least 5.0×10⁷ TCID50/mL of the Enterovirus A virus is harvested.

In some embodiments, an Enterovirus C virus of the present disclosure (e.g., poliovirus S1, S2, or S3) is harvested by lysing the host cells (i.e., the cells inoculated with and producing the virus). As described herein, a significant amount of virus produced may be found intracellularly in the production cells, and thus cell lysis may significantly increase viral harvest and yields. A variety of cell lysis methods are known in the art and suitable for a range of producer cells. In some embodiments, the cells are lysed by freeze-thawing. In some embodiments, the cells are lysed by a surfactant (including without limitation a polysorbate such as TWEEN®-80 or a polyethylene-glycol-based surfactant such as TRITON® X). In some embodiments, the cells are lysed by physical shearing.

### Devices for Cell Culture

Certain aspects of the present disclosure relate to methods for culturing a cell. In some embodiments, a cell is cultured in a device such as a fixed-bed, rocking, or stirring/stirred-tank bioreactor. Exemplary cell culture devices are commercially available and known in the art. See, e.g., the bioreactors described in U.S. Pat. Nos. 8,597,939, 8,137,959, US PG Pub 2008/0248552, WO2014093444, and Genzel, Y. et al. (2006) Vaccine 2:6074-87; bioreactors from Medorex; the WAVE or Xcellerex bioreactors from GE Healthcare Life Sciences; or the iCELLis® Bioreactors from Pall® Life Sciences, Port Washington, N.Y., such as the Nano, 500/100, and 500/66 bioreactors.

In some embodiments, the cell is cultured in a fixed-bed bioreactor. Fixed-bed bioreactors include a carrier in the form of a stationary packing material forming a fixed or packed bed for promoting cell adhesion and growth. The arrangement of the packing material of the fixed bed affects local fluid, heat, and mass transport, and usually is very dense to maximize cell cultivation in a given space. In one embodiment, the reactor includes a wall forming an interior with a packed or fixed bed comprised of a packing material (such as fibers, beads, spheres, or the like) for promoting the adhesion and growth of cells. The material is located in a compartment within the interior of the reactor, which compartment may comprise an upper portion of a hollow, vertically extending tube. A second compartment is provided within the interior of the reactor for conveying fluid to and from the material of compartment at least partially forming the fixed bed. Typically, the packing material should be arranged to maximize the surface area for cell growth, with 1,000 square meters being considered an advantageous amount of surface area (which, for example, may be achieved using medical grade polyester microfibers as the packing material). In one embodiment, evenly-distributed media circulation is achieved by a built-in magnetic drive impeller, ensuring low shear stress and high cell viability. The cell culture medium flows through the fixed-bed from the bottom to the top. At the top, the medium falls as a thin film down the outer wall where it takes up O₂ to maintain high K_{L}a, in the bioreactor. This waterfall oxygenation, together with a gentle agitation and biomass immobilization, enables the bioreactor to achieve and maintain high-cell densities. In some embodiments, the fixed bed has a bed height of about 2 cm. In other embodiments, the fixed bed has a bed height of about 10 cm.

In some embodiments, the fixed bed contains a macrocarrier (e.g., a matrix). In some embodiments, the macrocarrier is a fiber matrix. In some embodiments, the macrocarrier is a carbon fiber matrix. The macrocarrier may be selected from woven or non-woven microfibers, polyester microfibers (e.g., medical-grade polyester microfibers) porous carbon and matrices of chitosans. The microfibers may optionally be made of PET or any other polymer or biopolymer. In some embodiments, the macrocarriers include beads. The polymers may be treated to be compatible with cell culture, if such treatment is necessary.

Suitable macrocarrier, matrix or “carrying material” are mineral carriers such as silicates, calcium phosphate, organic compounds such porous carbon, natural products such as chitosan, polymers or biopolymers compatible with cells growth. The matrix can have the form of beads with regular or irregular structure, or may comprising woven or non-woven microfibers of a polymer or any other material compatible with cell growth. The packing can also be provided as a single piece with pores and or channels. The packing in the recipients can have a variety of forms and dimensions. In some embodiments the matrix is a particulate material of solid or porous spheres, flakes, polygons. Typically a sufficient amount of matrix is used to avoid movement of the matrix particles within the recipient upon use, as this may damage cells and may have an influence on the circulation of gas and/or medium. Alternatively the matrix consists of an element which fits into the inner recipient or into a compartment of the recipient, and having an adequate porosity and surface. An example hereof is a carbon matrix (Carboscale) manufactured by Cinvention (Germany). In some embodiments, the fiber matrix has a surface area accessible to the cell of between about 150 cm²/cm³ and about 1000 cm²/cm³. In some embodiments, the fiber matrix has a surface area accessible to the cell of between about 10 cm²/cm³ and about 150 cm²/cm³. For example, the fiber matrix may have a surface area accessible to the cell of about 10 cm²/cm³; about 20 cm²/cm³; about 40 cm²/cm³; about 60 cm²/cm³; about 80 cm²/cm³; about 100 cm²/cm³; about 120 cm²/cm³; about 150 cm²/cm²; about 200 cm²/cm³; about 250 cm²/cm³; about 500 cm²/cm³; or about 1000 cm²/cm³, including any value therebetween. In some embodiments, the fiber matrix has a surface area accessible to the cell that is less than about any of the following (in cm²/cm³): 1,000; 900; 800; 700; 600; 500; 400; 300; 200; 175; 150; 125; 100; 90; 80; 70; 60; 50; 40; 30; or 20. In some embodiments, the fiber matrix has a surface area accessible to the cell that is greater than about any of the following (in cm²/cm³): 10; 20; 30; 40; 50; 60; 70; 80; 90; 100; 125; 150; 175; 200; 300; 400; 500; 600; 700; 800; or 900. That is, the fiber matrix has a surface area accessible to the cell that can be any of a range (in cm²/cm³) having an upper limit of 1,000; 900; 800; 700; 600; 500; 400; 300; 200; 175; 150; 125; 100; 90; 80; 70; 60; 50; 40; 30; or 20 and an independently selected lower limit of 10; 20; 30; 40; 50; 60; 70; 80; 90; 100; 125; 150; 175; 200; 300; 400; 500; 600; 700; 800; or 900; wherein the lower limit is less than the upper limit. In some embodiments, the fiber matrix has a surface area accessible to the cell of about 120 cm²/cm³.

In some embodiments, the macrocarrier (e.g., a fiber matrix) has a porosity between about 60 and 99%. In some embodiments the macrocarrier (e.g., a fiber matrix) has a porosity between about 80 and about 90%. Optionally, the packing may have a porosity P in the range of 50% to 98%. The term porosity P is the volume of air present in a given volume of the material, and expressed as percentage of the given volume of the material. The porosity can be measured by measuring the weight Wx per volume of the porous material, and using the formula:

P=100−(1−W_{X}Wspec)

where Wspec is the specific weight of the material. The porous material may be one solid unit of porous material, or may be a plurality of individual units, such as grains, chips, beads, fibers or fiber agglomerates.

In some embodiments, the fixed bed is a single-use or disposable fixed bed. For example, commercially available bioreactors such as the iCELLis® Nano, 500/100, and 500/66 bioreactors Bioreactors (Pall® Life Sciences, Port Washington, N.Y.) may include a bioreactor system with a removable, disposable, or single use fixed bed that provides a large growth surface area in a compact bioreactor volume. Compared to a standard stirred-tank bioreactor using microcarriers, such systems avoid several delicate and time-consuming procedures, including manual operations, sterilization and hydration of microcarriers and bead-to-bead transfers from preculture to final process. As described herein, such bioreactors may enable process at a large scale (e.g., 500 square meters) culture area equivalent and harvest fluid volumes of up to 1500 to 2000 L, which is advantageous for industrial scale production of virus (e.g., for use in vaccine production). As exemplified herein, such devices may enable further advantages such as low cell inoculums; reaching of optimal cell density for infection at a short preculture period; and/or optimization of MOI, media and serum concentrations during the culture growth phase, such devices may be configured to allow rapid perfusion of the cells in culture, e.g., such that 90% or more of the cells experience the same medium environment. Moreover, and without wishing to be bound to theory, a single-use or disposable fixed bed may allow streamlined downstream processing to maximize the productivity as well as reduce the foot print of the process area even with scale up equivalent to several large scale conventional culture vessels. As such, advantageous productivity and purity may be achieved with minimal steps and costs.

In one embodiment, the recipient for cell cultivation has an inner space, which may be annular but may take other forms. The space contains packing. When the recipient is to be used for cell culture the packing should be compatible with ceil growth. In the annular configuration, the inner space has an annular volume delimited by: an outer tubular wall having a first outer end and a second outer end and a longitudinal wall extending in longitudinal direction. The outer tubular wall delimits an outer boundary of the annular volume in a longitudinal direction; a first and a second closure delimiting and closing the annular volume at the first outer end respectively the second outer end of the outer tubular wall; an inner elongate wall having a first outer end oriented towards the first outer end of the outer tubular wall, and a second outer end oriented towards the second outer end of the outer tubular wall. The inner elongate wall is positioned within the outer tubular wail. The inner elongate wall extends in a longitudinal direction and delimits an inner boundary of the annular volume, the inner boundary being encompassed by the outer boundary. The second outer end of the inner elongate wall coincides with the second closure. As an example, the outer tubular wall is provided by a cylindrical outer tubular element. The inner elongate wall may be provided by a solid inner cylindrical element, such as a cylindrical rod. The outer tubular element is a cylindrical tubular element, and has a central axis, parallel to the longitudinal direction. The inner cylindrical element and the outer tubular element may be coaxially mounted.

In some embodiments, the first outer end of the inner cylindrical element may comprise a coupling element to couple the inner cylindrical element, and by means of the closures being fixed to the inner cylindrical element and the outer tubular element, the outer tubular element as well, to a drive mechanism, e.g. a motor of the bioreactor. The second closure is provided with a connector, suitable to couple the recipient to a medium or gas source, for providing and/or extracting medium and/or gas to and/or from the inner space. This connector or alternatively additional connectors may be provided to the first closure or the second closure.

In some embodiments, upon moving the recipient, the packing, in particular the porous material, may rest in a fixed relative position to the recipient, or may move within and relative to the recipient or, as the case may be, within the compartment of the recipient. The recipient is to be rotated about its axis, optionally at a rotational speed of between 0.1 and 25 rotations per minute.

In some embodiments, the inner space is partially filled with cultivation medium, such as cell cultivation medium. As an example, the liquid level at least contacts the inner elongate wall, or the inner elongate wall is partially submerged in the medium. The part of the packing positioned under the liquid level is wetted by the cultivation medium, such as cell cultivation medium. The packing positioned above the liquid level is in contact with the gas or air present in the inner space. When the recipient is rotated in one direction about the axis, e.g. clockwise rotated, the cultivation medium, such as ceil cultivation medium rotates in opposite, say anti-clockwise direction relative to the packing. The cultivation medium, such as cell cultivation medium, is passed through the complete packing according to a plug flow. Upon rotation, e.g. clockwise, of the recipient, the cultivation medium, such as cell cultivation medium forces the gas or air at the leading edge of the plug flow to displace anti-clockwise. At the tailing edge of the medium, an optionally limited depression is created, causing gas or air to be sucked towards the trailing edge. As such the medium and the gas or air passes through the complete packing.

In some embodiments, the inner elongate wall of an alternative recipient may be provided by a cylindrical tubular element. The outer tubular wall is provided by an outer tubular element. The inner elongate wall is provided by an elongate cylindrical tubular element. The outer tubular element and the elongate cylindrical tubular element are fixed to two removable closures. The first closure is provided with a coupling element for coupling the recipient to a driving means for rotating the recipient along an axis in a longitudinal direction. The first closure further comprises a connector for connecting the inner space to a conduit, such as a flexible tube.

In some embodiments, the outer tubular element may be a glass tube, having a length L of e.g. 110 mm and an inner diameter Do of, for example, 135 mm. The inner elongate element may be a polyvinylidenefluoride (PVDF) tube having an outer diameter Di of, for example, 88.9 mm. The outer ends of the inner elongate element, hence of the inner elongate wail, coincide with the closures. The closures may be stainless steel or PVDF annular discs, which may be attached to the inner and outer element using silicone. The first closure, which may be provided with a connector, has a coupling element having an outer diameter Di of, for example, about 35 mm. The inner space is at least partially filled with packing.

In some embodiments, the recipient further comprises 2 fluid permeable dividers dividing the inner space in 2 compartments. The fluid permeable dividers extend from the inner elongate wall to the outer tubular wall and from the first closure to the second closure in the longitudinal direction parallel to the direction of the tubular axis. One compartment is provided with the packing. One compartment is not provided with the packing. The fluid permeable dividers may be provided with pores, such as by using porous material for providing porous dividers, or are provided with apertures, in any case allowing passage of liquid, i.e. cultivation medium, and gas. The dividers are however provided with pores or apertures small enough to prevent the packing to pass from one side of the divider to the other.

In some embodiments, the outer tubular wall is provided by an outer tubular element having a racial cross-section along a plane perpendicular to the longitudinal direction, which cross-section has the shape of a circle. The inner elongate wall is provided by an inner elongate element having a racial cross-section along a plane perpendicular to the longitudinal direction, which cross-section has the shape of a truncated circle or circular segment. In one embodiment the circle segment has a circle section and a chord. For example the height of the circle segment has the dimensions of 200 mm (Dec), 400 mm (Do), 240 mm (Di) and 125 mm (L). The dividers are in this embodiment coplanar with the chord of the circle segment. The second closure of the two closures is provided with two connectors. The first connector is provided near the outer tubular wall. The second connector is provided near the inner elongate wall. When the recipient is only partially filled with cultivation medium, which medium has a liquid surface, the recipient may be rotated to such a position that the liquid does not contact the inner elongate wail, but remains on a given distance from the inner elongate wall. When the recipient is brought in this position, the first connector may be used to remove or provide medium to the compartment, which is not filled with packing. Gas may be removed or provided above the medium liquid surface by means of connector. By rotation of the recipient either clockwise or counter clockwise, e.g. over an angle of up to 360° or even more, the medium will pass through to one of the two dividers, more particular through the divider which will gradually be submerged in the medium. The medium will slowly flow through the packing, as the packing gradually will pass through the medium because of the rotation. Due to the rotation of the recipient, the medium will pass and flow through the complete packing according to a plug flow. A uniform contact between medium and packing throughout the annular volume will occur. Once a part of the packing has passed through the medium, the medium will gradually seep out of the packing and hence the gas in the recipient may again contact the packing, allowing the cells to grow uniformly throughout the packing.

In an alternate embodiment of the recipient, the annular volume of the inner space is provided by a plurality of annular sections, in this particular case four annular quarters. Each of the sections provides one part of the outer tubular wall by means of an outer tubular wall section. Each of the sections provides one part of the inner elongate wall by means of an inner elongate wall section. Each of the sections has two radially extending section walls. These section walls are liquid and gas impermeable. Each of the section walls is provided with mounting means allowing adjacent sections to couple one to the other. A first and a second section closure delimit and close the volume of the annular sections at the first respectively the second outer end of the outer tubular wall. The section closures together form the first respectively second closure of the annular volume of the recipient.

In some embodiments, each of the sections may further be provided with two fluid permeable dividers, dividing the inner space of each annular section in three compartments. The fluid permeable dividers, e.g. porous dividers extend from the inner elongate wall to the outer tubular wall and from the first closure to the second closure in the longitudinal direction parallel to the direction of the tubular axis. One compartment is provided with the packing. Two compartments are not provided with the packing. For each annular section, a first connector is provided near the outer tubular wall. The second connector is provided near the inner elongate wall. Each of the sections may function as an independent recipient section of the recipient, when the recipient is rotated about the axis. The packing in each of the sections is provided with medium, which is present in this section depending upon the radial position of the section.

By mounting the sections, in this embodiment four sections, a recipient with an outer tubular wall and an inner elongate wall is obtained, of which the annular volume is closed at the two outer ends of the outer tubular wall by means of two closures. Because the coupling of the sections is provided by mounting and coupling two radially extending section walls, the combination of two contiguous section walls form a liquid and gas impermeable divider extending from the inner elongate wall to the outer tubular wall and from the first closure to the second closure in the longitudinal direction parallel to the direction of the tubular axis. This embodiment has the advantage that each of the sections may be provided with different medium for a different cell culture. Also, in case of an incorrect functioning of the packing of reaction in one of the sections, only one section is to be replaced. The rotation of the recipient (or any other recipient disclosed herein) may be provided by a rotator. In one example, this rotator may include contacting the outer surface of the outer tubular wall with at least two supporting wheels of which at least one is driven.

In an alternate embodiment, each of the sections of the recipient has two radially extending section walls. These section walls are liquid and gas permeable. Each section wall comprises a number of apertures, each of these apertures finding a corresponding aperture in a second section wall of an adjacent compartment. The aperture of the first section wall may be provided with an outwardly extending rim which extends through the corresponding aperture of the second section wall. Optionally a seal is provided around the apertures in the adjacent section walls to prevent medium from leaking between the contacting walls. In alternative embodiments, flexible tubing is placed between the recipients instead of seals.

In an embodiment of the bioreactor utilizing the recipient, at least one recipient is rotatably mounted in a vessel, which may have any suitable radial cross-section such as e.g. circular or polygonal such as rectangular (optionally square). The vessel is partially filled with cultivation medium, so the liquid level optionally does not raise higher than the axis of the bioreactor, but at least contacts the inner elongate wall. The recipient is rotated about this axis, which is identical to the longitudinal axis of the recipient. The longitudinal axis of the recipient is an axis parallel to the longitudinal direction of the outer tubular wall. Optionally the outer tubular wall is provided with apertures or is made from a porous, e.g. liquid and gas permeable material. When such fluid permeable outer tubular wall is rotated in a vessel partially filled with medium, so that only part of the outer tubular wall is submerged in the medium, the packing may be provided with medium flowing into the annular volume through the outer tubular wall. Part of the medium may be dragged along with the packing when the recipient is to rotate in the vessel.

In some embodiments, at least one of the recipients comprises a magnetic element. The bioreactor further comprises a magnetic element, which co-operates with the magnetic element of the recipient. Both magnetic elements are positioned such that the rotation of the magnetic element of the bioreactor around the axis of rotation will induce the rotation of the magnetic element of the recipient, hence will rotate the complete recipient. The adjacent recipients may be mounted on a common shaft, around which they rotate. The adjacent recipients may be coupled to each other in a fixed position, so the rotation of recipient induces the rotation of the recipient as well.

In some embodiments, the recipients have a liquid and gas impermeable outer tubular wall, which has connectors for connecting the recipient to a gas or medium storage by optionally flexible tubing. The recipients may be rotated by means of a driving system for providing a bioreactor. The recipient is mounted on a rotatable shaft, which is rotatable about an axis of rotation coinciding with the axis of the recipient. The shaft is profiled and fits in a unique rotational position within the inner void of the inner elongate element. As such, by controlling the rotational position of the shaft, the position of the recipient about the axis is unambiguously defined. The driving system may further comprise a motor, such as a linear motor, or any other suitable means to precisely control the rotation of the shaft. A clamp screw or any suitable means to prevent the recipient to displace in longitudinal direction over the shaft may fix the position of the recipient on the shaft in longitudinal direction. It is understood that optionally more than one recipient may be mounted on a common shaft. The shape of the inner void of the inner elongate element and the perimeter of the shaft are chosen such that the shaft and the recipient may be mounted in a limited or even in a unique way.

In one embodiment of the recipient, the inner elongate element has a longitudinal recess in the inner wall of the inner elongate element. A ridge on the shaft fits into this recess. The recipient fits in an unambiguous way on the shaft. The ridge may be slidingly moveable in the recess. In an alternate embodiment of the recipient, the inner elongate element has two longitudinal recesses in the inner wall of the inner elongate element. Two mutually perpendicular ridges on the shaft fit into these recesses. The recipient fits in two ways on the shaft, the first position being 180° rotated about the axis relative to the second position. The ridges may be slidingly moveable in the recesses. In a further embodiment of the recipient, the inner elongate element has four longitudinal recesses, one in each of the inner walls of the inner elongate element provided by a compartment. The shaft has a substantially cross-like cross-section, comprising four mutually perpendicular ridges on the shaft. Each of the ridges fits into a recess of one of the compartments. The recipient fits in four ways on the shaft. The ridges may be slidingly moveable in the recesses.

In a further embodiment of the bioreactor recipient, the volume of the inner space is provided by a plurality of segments, in this particular case four quarters. Each of the sections provides one part of the outer tubular wall by means of an outer tubular wail part. Each of the segments provides one part of the inner elongate wall by means of an inner elongate wall part. Each of the segments has two radially extending segment walls. As an example segment has two radially extending segment walls. The cultivation medium further may fill the inner void of the inner elongate wall. Through the apertures, the cultivation medium may flow in or out of the sections, and optionally to the adjacent section. The recipient includes a body forming a longitudinal wall and ends in the form of caps. The caps may be removable, and in the connected position form a fluid-tight seal for containing any fluid within the body. Each cap may include an opening forming an inlet or outlet for receiving the culture medium, but the inlet and outlet could each be provided in the same cap as well, or in the longitudinal wall of the body. At least one or a pair of fluid-permeable structures, such as perforated partitions, are provided forming a compartment for containing any packing. The perforations in each partition may be provided in a shape and size to control the flow and residence time of the fluid in the compartment, and may be the same or different among the partitions. The packing may be provided in a manner such that it completely occupies the compartment of the recipient, and thus circumferentially contacts the inner surfaces of the wall of the body, as well as the fluid-permeable structures.

In some embodiments, the recipient may be associated with a rotary device. The device may include a pair of rollers for receiving, supporting, and rotating the recipient about the longitudinal axis of the body. The recipient may be provided with a generally cylindrical body adapted for engaging and being rotated by the rollers to help distribute any fluid (e.g., the culture medium, or any rinsing or recovery agent) through any packing present in the compartment. Tubular connectors may also be provided in association with the inlet and outlet for delivering fluid to the compartment. This may be done while the recipient is stationary or while it is rotating. In the latter case, the connectors may be connected in a manner that permits relative rotation, such as by using a rotary joint created by way of a snap-fit engagement or using a bearing. Seals, such as O-rings, may be used to help prevent any leakage and help maintain the sterile conditions desirable for cell culturing. One advantage of the recipient is the simplicity of the arrangement. For example, the recipient includes no sensors, probes, mixers, or the like, in the event it is desirable to include such structures, this is possible, and may be accomplished by connecting the recipient in a closed loop with a reservoir. The reservoir may include any number of sensors or the like for measuring one or more characteristics of the circulated fluid, and may include a single use vessel (such as a flexible bag) to avoid the need for cleaning and sterilization. A pump, such as a peristaltic pump, may also be provided for circulating the fluid through the loop.

In this embodiment, the recipient may be constructed according to any of the above details (thus forming a roller bottle), and includes an inlet and an outlet. Each of the inlet and outlet may be connected to a conduit that permits rotation of the recipient without unduly biding or twisting the conduit in a manner that does not interfere with the fluid transmission, and thus may allow for the continuous flow of fluid to and from the recipient while it is rotated. Specifically, the conduit may comprise a coiled tube having an open end for connecting to the inlet or outlet, respectively, and may at the opposite ends associate with any fluid reservoir. A suitable pumping arrangement may also be provided for moving fluid through the conduit and the recipient.

In one embodiment, the recipient may be rotated in a first direction, such as clockwise, for one or more complete rotations, using a suitable rotator (such as rollers). The number of rotations possible without binding of the conduit may vary, but it is envisioned that 2-3 rotations should be possible at a minimum. The recipient may then be rotated in a second, opposite direction for one or more complete rotations. More specifically, the rotation may be for the number of rotations in the first direction to return the coiled tube to its home position, plus a corresponding number of rotations in the second direction for so long as the coiled tube does not bind or otherwise interfere with the fluid transmission. The rotation and counter-rotation may occur continuously or intermittently, and the same is true for the delivery and recovery of fluid via the conduit. It can also be understood that, in the event the recipient includes a sensor, it may also be connected to any source of energy used for sensing. In such case, any transmission line, such as a cable, may also be coiled or spiral to accommodate the relative rotation in the manner contemplated above without twisting or binding. Again, it is desirable to place any sensors or the like in any recirculation loop, though, since this drives down the cost of the recipient.

In a further embodiment of a recipient in the form of a roller bottle, the inlet and outlet may be provided in a common wall. The inlet may also be associated with a tube positioned within a fluid permeable internal cylinder within the fixed bed, which helps to ensure the fluid introduced (gas, liquid, or both) does not simply immediately exit through the inlet. A gas may be introduced into a liquid by placing an injector, such as a rotameter, in the recirculation loop. The placement may be immediately upstream of the inlet. This manner of gas introduction in connection with liquid flow advantageously helps to reduce the incidence of foaming and improve the mass transfer rate, since the gas remains in pockets separated by the liquid. The transmission line associated with the outlet returns to the reservoir, which may be vented through a filter as shown, to avoid a vent for direct connection with the recipient.

In some embodiments, other elements may be added to the recipients and the bioreactors. As an example the inner elongate wall may be an elongate tubular, optionally cylindrical wall. The inner void of the inner tubular wall may be used to accommodate one or more sensors, such as temperature sensors, position sensors (e.g. for defining the orientation of the recipient relative to the axis of rotation), optical sensors (e.g. for generating data on the color of the cultivation medium, such as cell cultivation medium), pH-sensors, oxygen sensors (such as Dissolved Oxygen (DO)-sensors), CO₂-sensors, ammonia sensors or cell biomass sensors (e.g. turbidity densitometers). Such sensors may additionally or optionally be located in or on the closures. The recipient may also accommodate perfusion, continuous addition of fresh nutrient medium and the withdrawal of an equal volume of used medium, allowing the realization of cell cultivation conditions that are approximated as closely as possible to the in vivo situation. The combination of a perfusion ceil culture with e.g. an enzyme glucose biosensor allows the glucose consumption of the cell culture to be monitored continuously. It is also understood that heating elements, such as heating blankets, may be provided to the outer and optionally the inner wall for heating or maintaining the temperature of the medium and the packing in the recipient.

In an alternate embodiment, the bioreactor comprises a cell culture vessel comprising a substantially vertical and cylindrical culture vessel, although other forms can also be envisaged, for example any prismatic shape, preferably regular. The culture vessel comprises at least four zones in communication with one another. From the center of the vessel towards the outside, the vessel comprises a first zone, a third zone, second zone and a fourth zone.

In some embodiments, the culture vessel comprises medium circulation means in its bottom part. The medium circulation means are, in this preferential embodiment, composed of a magnetic device, for example a magnetic bar in rotation about a central rotation axis, real or virtual, a first end of which is housed in a top engagement means and a second end of which is housed in a bottom engagement means. The magnetic bar is driven by a rotary magnetic drive motor external to the culture vessel and which is not shown here. The circulation means comprise at least one medium inlet. The medium inlet comprises at least one first end which ends in a diversion baffle for the flow of medium. The magnetic bar functions as a centrifugal pump, that is to say the medium is sucked into a relatively central zone by the movement of the medium created by the bar and the medium is propelled outwards with respect to the central point. The medium diversion baffle guides the medium in the relatively central zone of the bar so that the medium is sucked therein and is then propelled outwards. Advantageously, the inlets are in the same plane (star configuration) and the number of inlets will be a number such that their positions will exhibit symmetry. More particularly, if three inlets are considered, it is advantageous for them each to be separated from one another by an angle of approximately 120, if the number of inlets equal 4, the inlets will be separated from one another by an angle substantially equivalent to 90°, if the number of inlets is equal to 10, the inlets will be disposed with a separation angle approximately equal to 36°. The medium circulation means also comprises at least one medium outlet. The medium outlet is advantageously situated at the point where the medium is propelled by the centrifuge effect of the magnetic bar. Advantageously, the number of outlets will be a number such that their positions will exhibit symmetry. More particularly, if three outlets are considered it is advantageous for them each to be separated from the other by an angle of approximately 120°, if the number of outlets is equal to four, the outlets will be separated from one another by an angle substantially equivalent to 90°, if the number of outlets is equal to 10, the outlets will be disposed with a separation angle of approximately 36°. Preferably, the outlets are not situated in the same horizontal plane as the inlets. The bottom part of the culture vessel comprises at least one medium guiding means, adjacent to said at least one outlet, which guides the culture medium propelled towards to the top of the culture vessel.

In some embodiments, the first zone of the culture vessel is a substantially central zone and is a medium transfer zone. The first zone comprises a basal part and in particular embodiments optionally also a cylindrical part. The diameter of the basal part is less than the diameter of the culture vessel. The basal part is in medium communication with said at least one medium outlet of the medium circulation means. The basal part is reduced in the top part of the first zone to a cylinder with a smaller diameter than the basal part. The top cylindrical part comprises an external wall and is in direct medium communication with the basal part of said first medium transfer zone. The third zone is a medium transfer zone, external to the first medium transfer zone. The third zone also comprises a substantially basal part (in the form of a sleeve) and in particular embodiments optionally also a substantially cylindrical top part.

In some embodiments, the substantially cylindrical part of the third medium transfer zone is essentially concentric with the substantially cylindrical part of the first medium transfer zone and these two parts are in medium communication. The medium communication is achieved by means of an orifice or a tube, by overflowing (as shown in the figure) via overflow or any other possible means for achieving this communication. The second zone is a cell culture zone, with or without carriers or microcarriers. The second zone is also in the form of a sleeve, at the center of which are the first and third medium transfer zones. The second zone comprises a bottom wall and a top wall, each wall and being provided with orifices allowing a transfer of cultured medium essentially free from cells. The second culture zone is in medium communication with the relatively basal part of the third medium transfer zone by means of orifices in the bottom wall allowing the medium to pass. The fourth zone is a medium transfer zone, external to the second culture zone but internal to the culture vessel. The fourth zone is in medium communication with the second culture zone. It is also in medium communication with the medium circulation means, via said at least one inlet. The medium communication is achieved by means of an orifice or a tube, by overflowing or by any other possible means for achieving this communication.

In some embodiments, the culture device comprises a substantially cylindrical culture vessel, but other embodiments can also be envisaged, as mentioned previously, for example a substantially prismatic vessel, preferably regular. Obviously, this is also the case with the various medium and culture transfer zones. They can also be prismatic, preferably regular, any combination of shapes being possible. In this case, the term sleeve must be envisaged as an envelope with a cross-section similar to the cross-section of the prism envisaged. When the medium circulation means are in operation, the medium leaves them through said at least one outlet, when there are several of them, through the various outlets, and is diverted by the guiding means, it ends up in the substantially basal part of the first medium transfer zone. The structure of the first medium transfer zone and the output of the pump require the medium to be directed towards the substantially cylindrical part of the first medium transfer zone. When it reaches the top of the wall of the substantially cylindrical part, it overflows via overflow into the third medium transfer zone.

It is clear to the skilled in the art that, in this particular embodiment, the wall of the substantially cylindrical part of the first medium transfer zone is less high than the wall of the third medium transfer zone for reasons of efficiency and flow rate, but he will easily understand that the wall of the substantially cylindrical part of the first medium transfer zone can also be higher than the wall of the substantially cylindrical part of the third medium transfer zone. The medium is therefore subjected to the flow rate imposed by the pump and to gravity, it is directed downwards from the third medium transfer zone running down the substantially cylindrical part and reaches the substantially basal part of the third medium transfer zone. Next the flow of medium has a rising direction through a communicating vessels effect by the imposed flow rate of the pump and reaches the top of the second culture zone. The medium reaches the second culture zone from the third medium transfer zone via the orifices for the passage of medium substantially free from cells in the bottom wall of the second culture zone. As already mentioned, the medium passage orifices are sized according to the type of culture. If the culture is a culture without carrier, the wall comprising orifices will be a porous membrane where the pore size is less than the diameter of the cells. If the culture is on microcarriers or on carriers, the size of the orifices will be less than the size of the microcarriers or carriers. When the medium flow edge reaches the top of the wall of the second culture zone, it overflows into the fourth medium transfer zone. Naturally, if orifices are present or a tube, it must be understood that, when the medium flow edge reaches the orifice or tube, it flows into the fourth zone.

In one embodiment, the fourth medium transfer zone comprises an inclined wall on which the medium flows when it passes from the second zone to the fourth zone. The inclined wall preferably comprises a hydrophilic membrane in order to improve the formation of the film on said inclined wall. The film must preferably be laminar in order to prevent as far as possible the formation of foam. In order to stabilize the film, it is also possible to add additives to the culture medium in order to modify the rheological properties of the water, in particularly of the culture medium, such as the additives included in the group consisting of surfactants, Pluronic F68, glycerine, quaternary ammoniums and any other additive for modifying the rheological properties of the culture medium. The hydrophilic membrane will for example be a membrane consisting of polyoxyethylene. The formation of the film on the inclined wall is an important step since it allows oxygenation on “thin film”. Indeed the gaseous volume with respect to the quantity of medium in this fourth medium transfer zone is large and improves exchanges. In addition, the formation of the film on an inclined wall increases the gas-liquid contact surface area.

In some embodiments, the culture vessel preferably comprises a cover through which at least one gas inlet orifice and at least gas outlet orifice pass. The gas inlet orifice is preferably situated so as to communicate directly with the fourth medium transfer zone. In some variants, it may be preferable for the gas inlet orifice to be present on the vertical wall of the culture vessel or at the bottom of the culture vessel, that is to say the gas passes by means of an orifice through the wall of the culture vessel opposite to the cover, and for this orifice to be provided with a tube in order to end above the liquid level.

In this embodiment, the cover is fixed by fixing means to the top wall of the second culture zone. In variants, the cover can be made an integral part of the top wall of the second culture zone, this part opening when the cover of the culture vessel is raised. In this way, it is easy to take off a cell sample with or without carriers in order for example to evaluate the cell density, the structure of the cells and other physical characteristics of the cell which reflect the health of the culture. Indeed, connecting the two together makes it possible to open the culture compartment simply by raising the cover of the culture vessel. In the case of culture in suspension, it could be advantageous to connect a porous membrane to the top wall provided with orifices of the second culture zone, this assembly can improve the rigidity of the cover/membrane assembly for taking samples.

In some embodiments, the magnetic bar has the shape of a helix. The design of the magnetic device with a substantially central rotation axis will depend essentially on the volume of the culture. Indeed, for small cultures, the device sets out to be able to us a simple bar such as a magnetic chip for circulating the medium. For large volumes, the device envisages a magnetic rotor, also driven by an external motor, for example rotors like the ones used in aquariums which allow high medium circulation rates.

Some embodiments of the cell culture device can be envisaged using bubble production devices, more commonly referred to as “spargers” or “microspargers” according to the size of bubble produced. Advantageously, when bubbles will be used, the pierced end of the bubble production device, for example of the tube, will be immersed in the medium at the bottom of the fourth medium transfer zone or in the first medium transfer zone. When this type of oxygenation is chosen, it is always also possible to continue the oxygenation on thin film, which makes it possible to reduce the flow of gas and to form fewer bubbles and therefore to reduce the formation of foam. In this case, provision is also made for having two gas inlets in the cover of the culture vessel or on the vertical wall of the latter. In addition, it is also possible to envisage that the bubble production device be present solely as an SOS procedure, and used solely when necessary.

In some embodiments, the culture device also comprises a series of culture parameter sensors, for example for the dissolved oxygen partial pressure pO2, acidity pH, temperature, cloudiness, optical density, glucose, CO2, lactate, ammonium and any other parameter normally used for monitoring cell cultures. These sensors are preferably optical sensors which do not require connections between the inside of the culture vessel and the outside thereof. The preferential position of these sensors is a critical position in that it is advantageous for these to be situated close to the wall of the culture vessel, for them to be in contact with the medium and preferably in strategic positions, as in the zone through which the medium passes before it passes through the cells or just after.

In some embodiments, the cell culture device comprises a disposable bioreactor for all the reasons of simplicity and economy mentioned previously. Consequently, this is why the connections between the inside and the outside of the culture vessel have been reduced. In addition, the bioreactor comprises a particularly reliable bioreactor in which the risks of contamination are particularly low by being disposable.

An embodiment of a device also envisages a modular design which comprises a series of modules for cultures on a larger volume. For example, with this type of modular design, culture volumes of around 500 ml to 100 liters are for example envisaged, through the use of a very limited number of standard modules. In some embodiments the device provides a series of modules that can be “slipped” around the first medium transfer zone to be placed in a standard culture vessel comprising medium circulation means and a cover. In a particular variant, the device comprises a mounting system which comprises various standard modules. These standard modules are for example a circulation means module to be placed at the bottom of the assembly, one or more culture modules and a cover module. Although other means of fixing these modules can be envisaged, the modules will be clamped on one another, for example by means of rapid connectors perfectly impermeable from the liquid and gaseous point of view.

Consequently, according to the type of culture and the required volume, the user will be able to take from the stock a base module comprising the medium circulation means, he will also have to take therefrom the number of culture modules that he requires according to the required culture volume and then take a head module corresponding to the cover. Next, all these modules being packaged in sterile fashion, he will merely need to unpack them and “clip” them one above the other. The stacking can form the “disposable bioreactor” or can be placed in an appropriate vessel.

In some embodiments, the base module comprising the circulation means can be fixed to the bottom of the culture vessel can also be slid into the culture vessel in order to be able to dispose it and to use another one for another culture and thus prevent cross contaminations. These circulation means comprise a magnetic device, rotating about a central rotation axis, a first end of which is housed in a top engagement means and a second end of which is housed in a bottom engagement means. The circulation means comprise at least one medium inlet. The medium circulation means also comprise at least one medium outlet. The base module of the culture vessel comprises at least one medium guiding means adjacent to said at least one outlet, which guides the culture medium propelled towards the top of the culture vessel.

In some embodiments, the culture vessel comprises a series of culture modules which are stacked one above the other. It could also be envisaged that they be simply adjacent to one another, that is to say placed side by side. In some embodiments, the modules are clamped to one another by means of rapid connectors or clips. In addition, it may be advantageous for each module to comprise a gas or gas mixture inlet in communication with the fourth zone of each culture module. The vessel may also comprise for its part an outlet for the excess gas or gas mixture. For example, the gas inlet orifice may be present at the bottom of the culture vessel, that is to say the gas passes by means of an orifice through the wall of the culture vessel opposite to the cover and this orifice is provided with a tube in order to end above liquid level of the module. Consequently, the gaseous mixture reaches the fourth medium transfer zone of this module. The module placed above the module can also comprise a tube which enables the gaseous mixture present in the fourth zone of the culture module to communicate with the fourth zone of the module. This tube therefore advantageously passes through the bottom wall of the module.

In certain embodiments, for long duration culture, it may be advantageous to replace part of the culture medium with fresh medium or to carry out an addition of nutriment. Consequently, the base module can then comprise a nutriment inlet. Also advantageously, the culture vessel can comprise, at the medium circulation means, a medium outlet in order to prevent overflowing. In a similar manner, the culture vessel comprises a head module comprising a cover, advantageously connected to a top wall provided with medium passage orifices by fixing means in order to simplify taking samples in the module situated above. In addition, advantageously culture parameter sensors can also be provided in each culture module. It is also possible to provide sensors in only one or several culture modules at all zones or in the base module.

In some embodiments or the base module, the medium is propelled from the medium circulation means via said at least one outlet, when there are several of them, through the various outlets and is diverted by the guiding means. It ends up in the substantially basal part of the first medium transfer zone. The substantially basal part of this embodiment is a zone common to all the culture modules and, in the embodiment illustrated, is situated in the base module. This is valid whether the culture modules are stacked or juxtaposed. The structure of the first medium transfer zone and the output of the pump require the medium to be directed towards the substantially cylindrical part of the first medium transfer zone of the first module towards the substantially cylindrical part of the first medium transfer zone of the second module. In this embodiment, it is the assembling of the modules which creates a large first medium transfer zone comprising a substantially cylindrical part. When the medium reaches the top of the wall of the substantially cylindrical part of the second culture module, it overflows into the third medium transfer zone of the second culture module.

In some embodiments, the medium is therefore subjected to the flow rate imposed by the pump and to gravity, it is directed towards the bottom of the third medium transfer zone of the second culture module, flowing down the substantially cylindrical part of the second culture module, and reaches the substantially basal part of the third medium transfer zone of the second culture module. Next, the flow of medium has a rising direction through a communicating vessels effect and through the imposed flow rate of the pump and reaches the top of the second culture zone of the second culture module. The medium reaches the second zone of the second culture module from the third medium transfer zone of the second culture module via the orifices for the passages of medium substantially free from cells of the bottom wall of the second culture module. When the medium flow edge reaches the top of the external wall of the second culture zone of the second culture module, it overflows into the fourth medium transfer zone of the second culture module. Naturally, if orifices or a tube are present in this external wall of the culture zone, it is necessary to understand that, when the medium flow edge reaches the orifice or tube, it flows into the fourth zone of the second culture module. In the particularly preferential embodiment of the device, the fourth medium transfer zone of the second culture module comprises an inclined wall on which the medium flows when it passes from the second zone of the second culture module to the fourth zone of the second culture module. The inclined wall preferably comprises a hydrophilic membrane in order to improve the formation of the film on said inclined wall. The film must preferably be laminar in order to prevent as far as possible the formation of foam. In order to stabilize the film, it is also possible to add additives to the culture medium in order to modify the rheological properties of the water, as mentioned before. Next, the culture medium present in the fourth medium transfer zone of the second culture module overflows either through a tube or over the top of the wall of the fourth medium transfer zone of the second culture module into the third medium transfer zone of the first culture module.

In some embodiments, the medium is subjected to the flow rate imposed by the pump and to gravity, it is directed downwards from the third medium transfer zone of the first culture module, flowing down the substantially cylindrical part of the first culture module, and reaches the substantially basal part of the third medium transfer zone of the first culture module. Next, the flow of medium has an upward direction through a communicating vessels effect and through the flow rate imposed by the pump and reaches the top of the second culture zone of the first culture module. The medium reaches the second zone of the first culture module from the third medium transfer zone of the first culture module via the orifices for passage of medium substantially free from cells in the bottom wall of the first culture module. When the medium flow edge reaches the summit of the wall of the second culture zone of the first culture module, it overflows into the fourth medium transfer zone of the first culture module. Obviously, if orifices or a tube are present in this wall, it must be understood that, when the medium flow edge reaches the orifice or the tube, it flows into the fourth medium transfer zone of the first culture module. The fourth medium transfer zone of the first culture module can also comprise an inclined wall on which the medium flows when it passes from the second culture zone of the first culture module to the fourth medium transfer zone of the first culture module. The inclined wall is possibly provided with a hydrophilic membrane as above. Next, the medium returns to the base module and to the medium circulation means through the inlet (pipe), that is to say the culture medium present in the fourth medium transfer zone of the first culture module overflows either via a tube or over the top of the wall of the fourth medium transfer zone of the first culture module in a pipe which ends in a substantially central zone of a siphon created by said centrifugal pump which constitutes the medium circulation means.

In a variant of this embodiment, the stacked modules constitute the culture vessel. In this variant, there can exist for example three kinds of module, base modules, modules comprising the four zones, and a head module mx. The base module or basal module comprises medium circulation means and assembly means; it is designed to engage the first assembly means of a four zones module as explained above, and to constitute the bottom of the vessel. The head module is designed to engage the second assembly means of a four zones module. The four zones module engaged by the base module can be the same as that engaged by the head module or the four zones module engaged by the base module can be the first in a series of four zones modules and the one engaged by the head module is consequently the second four zones module in said series of four zones modules.

In some embodiments, all the modules comprise fixing means which makes it possible to obtain a single culture module which can be assembled both with another culture module and with the base module or the head module. These fixing means are for example two concentric circles provided with a circular seal, rapid connectors well known in the art of cell culture, a screw pitch and a serration or any other device for assembling these modules.

In this embodiment, the basal part of the base module is bond with orifices substantially tubular in shape which are orifices allowing in this case an introduction of gas or gas mixture. The gas inlet orifice is connected to a tube which ends above the level of the culture medium, enabling the gas or gas mixture to reach at least a fourth medium transfer zone of the culture device. All the ambient atmospheres of the fourth medium transfer zone are connected by similar tubes so that the gas mixture can reach the top. It is particularly advantageous in a device with modules stackable for height which can rise very high to provide a gaseous supply through the bottom of the reactor. In a variant, the basal part comprises a gas or gas mixture feed tube for bringing the gaseous substance into the zone in which the magnetic device is situated. In this way, the incoming gas is stirred by the rotation of the magnetic device and the dissolution of the oxygen is improved by the movement of the medium. The excess gas is also stirred and moves upwards again in the form of small bubbles. In addition, a recess is provided for accessing these orifices from the outside, which makes it possible to connect these orifices to a supply of gas, gas mixture, fresh medium, etc. The top part mob of the module is an element designed to be gripped by virtue of the fixing means and sealingly by virtue of the circular seal on the bottom part of the base module.

In some embodiments, the device also comprises a nutriment feed, either in a tube through the cover, or a tube through one of the walls of the device. Likewise, heating means can also be present in the first or fourth zone of the device or of a module or each four zones module. Possibly, the device can also comprise several medium circulation means, for example several centrifugal pumps. The devices enable a homogenous flow of culture medium upon entry trough the orifices in the bottom part of the second zone and consequently also during the further passage through this second zone. This in contrast to devices wherein the first zone is in direct contact with the second zone which results in a non-homogenous flow throughout the second zone. Such non-homogeneous flow results in the present of undersupplied or dead zones within the cell culture zone which are insufficiently supplied with oxygen and nutrients, and wherein cell growth and/or metabolism is far from optimal.

A particular embodiment of the devices and methods relates to devices and their use, wherein the third zone is a zone internal to said second zone and external to said first zone. The flow of the medium from the basal part of the first zone upwards via the top cylindrical part of the first zone, further downwards via the cylindrical top part of the third transfer zone to the basal part of the third zone generates the desired homogeneous flow upon entry in the bottom part of the second zone. The presence of the cylindrical parts and further allows an easy access to the medium for assaying its properties prior to entry in the second zone. The presence of a cylindrical element also prevents that a high pressure is built up in the device. The presence of cylindrical parts allows in addition the manufacture of a device comprising different modules.

Other embodiments relate to devices which are modified such in that the liquid flow through the cylindrical parts is bypassed. In these alternative embodiments of devices the third zone is a located entirely below the second zone and entirely above the first zone. In one embodiment of the device the part of the device corresponding to the cylindrical parts of the first zone and the second zone is replaced by a solid element in e.g., a plastic, glass or metal.

In another embodiment, the first zone and the second zone consist of a flattened shaped volume corresponding respectively to the basal parts and lack the cylindrical parts. With this adaptation the culture medium can equally overflow from the first zone to the third zone via the overflow. The flow of the medium created by the stirring device is rendered homogeneous by the separating wall between first zone and third zone and results in a homogeneous flow upon entry of the second zone.

In particular embodiments, the separating wall between the first zone and the third zone consist of a horizontal part as well as a of a vertical part, wherein this vertical part with the overflow has a height of about up to 5%, up to 10%, up to 20% or even up to 50% of the height of the third zone. In other particular embodiments, the vertical part of the separating wall between the first zone and the third zone is absent.

In particular embodiments the solid element is provided with channels adapted to incorporate for example a probe to measure a condition of the medium in the third zone (pH, oxygen, temperature). In other particular embodiments, solid element is provided with a channel comprising a safety pressure valve which can open when an excessive pressure is built up in the first zone and third zone. In an alternative embodiment of the device the cylindrical parts of the respectively the first zone and the third zone second zone are absent. The volume previously occupied by elements now becomes parts of the second zone resulting in more efficient use of the device resulting from the enlarged volume which is suitable for cell growth.

In order to prevent the direct inflow of medium from the first zone into the second zone without a homogenous flow distribution into the third zone, the orifices in the bottom wall of the second zone are closed at those regions which are located above an opening in the wall between the first and the third zone. The adaptation of the device by providing a closed region above the opening results in the overflow of the culture medium from the first culture medium transfer zone into the third culture medium transfer zone before it enters as a homogenous flow into the second zone.

In particular embodiments, a plurality of openings and corresponding closed regions is provided into respectively the separating wall between first zone and third zone, and into the wall between third zone and second zone. Typically such plurality of openings and corresponding closed regions are distributed symmetrically.

In an alternative embodiment of the device, the homogenous flow of the medium is achieved by providing a flow redistributing element within the third zone. Such element can have any shape suitable for an appropriate redistribution of the medium coming from the first zone to obtain a homogenous liquid flow in third zone prior to entry in the second zone. In a particular embodiment the element has the form of a set of radially extending rods with a circular, diamond or oval cross section, positioned above corresponding radially applied openings.

### Downstream Virus Purification

In some embodiments, a method of the present disclosure for producing an Enterovirus C virus (e.g., poliovirus S1, S2, or S3) includes culturing an adherent cell in a fixed bed comprising a matrix, where the cell is cultured in a first cell culture medium; inoculating the cell with the Enterovirus C virus in a second cell culture medium under conditions in which the Enterovirus C virus infects the cell and the infected cell produces the Enterovirus C virus; and harvesting the Enterovirus C virus produced by the cell. In some embodiments, polysorbate is added to the second cell culture medium during inoculation of the cell with the Enterovirus C virus or from approximately one hour to approximately four hours prior to step (c). In some embodiments, the cell is inoculated with the Enterovirus C virus at an MOI of between about 0.01 and about 0.0009. In some embodiments, between about 150,000 cells/cm² and about 300,000 cells/cm² are inoculated. In some embodiments, the cell is cultured during steps (a) and/or (b) at a volume/surface ratio of about 0.1 mL/cm² to about 0.3 mL/cm². In some embodiments, step (b) further comprises culturing the inoculated cell under conditions in which the Enterovirus C virus infects the cell and the infected cell produces the Enterovirus C virus, and step (c) comprises lysing the cell to harvest the Enterovirus C virus produced by the cell. In some embodiments, the cell is inoculated at a pH that ranges from about 6.8 to about 7.4. In some embodiments, the method further includes passing the harvested Enterovirus C virus produced by the cell through a depth filter to produce a first eluate, where the first eluate comprises the Enterovirus C virus; binding the first eluate to a cation exchange membrane to produce a first bound fraction, where the first bound fraction comprises the Enterovirus C virus; eluting the first bound fraction from the cation exchange membrane to produce a second eluate, where the second eluate comprises the Enterovirus C virus; binding the second eluate to an anion exchange membrane to produce a second bound fraction, where the second bound fraction comprises the Enterovirus C virus; and eluting the second bound fraction from the anion exchange membrane to produce a purified Enterovirus C virus. In some embodiments, a method of the present disclosure for producing an Enterovirus C virus includes culturing a cell in a first cell culture medium; inoculating the cell with the Enterovirus C virus in a second cell culture medium under conditions in which the Enterovirus C virus infects the cell and the infected cell produces the Enterovirus C virus; and harvesting the Enterovirus C virus produced by the cell, wherein polysorbate is added to the second cell culture medium during inoculation of the cell with the Enterovirus C virus or from approximately one hour to approximately four hours prior to step (c).

In some embodiments, a harvested Enterovirus C virus of the present disclosure (e.g., poliovirus S. S2, or S3) is passed through a depth filter to produce a first eluate, which contains the virus. As is known in the art, depth filtration may be used to separate production cells, cellular debris, and other agents from Enterovirus C (e.g., produced by the cultured cells and/or harvested from the cultured cells by cell lysis), providing clarification for the harvested virus. Depth filters may be applied in a cartridge or capsule format, such as with the SUPRACAP™ series of depth filter capsules (Pall Corporation) using a Bio 20 SEITZ® depth filter sheet. Other suitable depth filtration techniques and apparatuses are known in the art. In some embodiments, the depth filter has a pore size of between about 0.2 μm and about 3 μm. In some embodiments, the pore size of the depth filter is less than about any of the following pore sizes (in μm): 3, 2.8, 2.6, 2.4, 2.2, 2.0, 1.8, 1.6, 1.4, 1.2, 1.0, 0.8, 0.6, and 0.4. In some embodiments, the pore size of the depth filter is greater than about any of the following pore sizes (in μm): 0.2, 0.4, 0.6, 0.8, 1.0, 1.2, 1.4, 1.6, 1.8, 2.0, 2.2, 2.4, 2.6, or 2.8. That is, the pore size of the depth filter can be any of a range of pore sizes (in μm) having an upper limit of 3, 2.8, 2.6, 2.4, 2.2, 2.0, 1.8, 1.6, 1.4, 1.2, 1.0, 0.8, 0.6, and 0.4 and an independently selected lower limit of 0.2, 0.4, 0.6, 0.8, 1.0, 1.2, 1.4, 1.6, 1.8, 2.0, 2.2, 2.4, 2.6, or 2.8; wherein the lower limit is less than the upper limit.

As described herein, cation exchange and anion exchange chromatography may be used in the methods of the present disclosure to purify an Enterovirus C virus (e.g., poliovirus S1, S2, or S3) harvested from a cell of the present disclosure. Advantageously, as demonstrated herein, the combination of these chromatography techniques allows for high yield and purity of harvested virus. For example, clarified viral harvest may be acidified, loaded onto a cation exchange membrane, eluted by salt or pH, filtered, basified, loaded onto an anion exchange membrane, eluted by salt or pH, filtered, and inactivated. This is only an exemplary scheme, and one of skill in the art may readily contemplate variants thereof with substituted, deleted, inserted, or reordered steps.

Anion and cation exchange chromatography both rely on the attraction of charged macromolecules of interest (e.g., a virus) in a mobile phase to a substrate having an opposite charge. In cation exchange chromatography, the negatively charged substrate or membrane attracts positively charged macromolecules. In anion exchange chromatography, the positively charged substrate or membrane attracts negatively charged macromolecules. Once macromolecules are bound or loaded onto the substrate, they may be eluted in linear or step-wise fashion from the substrate in a manner dependent on their characteristics, thereby enacting a separation of differently charged molecules. This principle may be used to purify viruses from other macromolecules. Elution may be effected by varying pH or salt content of the mobile phase buffer. As demonstrated herein, particular loading and elution parameters such as pH and salt content have dramatic effects on yield and purity of Enterovirus C virus purification. A variety of suitable buffers are known in the art and described herein. Viral purification methods using ion exchange chromatography are also generally known; see, e.g., purification of influenza virus available online at https://www.pall.com/pdfs/Biopharmaceuticals/MustangQXT_AcroPrep_USD2916.pdf.

Cation exchange chromatography may be used to purify an Enterovirus C virus of the present disclosure. As demonstrated herein, the buffers and conditions used for cation exchange chromatography loading (e.g., binding to a cation exchange membrane) and elution greatly impact virus purity and yield. In some embodiments, an eluate containing an Enterovirus C virus of the present disclosure (e.g., poliovirus S1, S2, or S3) is bound to a cation exchange membrane to produce a bound fraction containing the virus. In some embodiments, the eluate has been subject to depth filtration. In some embodiments, an eluate containing an Enterovirus C virus of the present disclosure is diluted (e.g., using a 2×, 3×, 4×, or 5× dilution factor) prior to cation exchange chromatography. In other embodiments, an eluate containing an Enterovirus C virus of the present disclosure is not diluted prior to cation exchange chromatography. In some embodiments, an eluate containing an Enterovirus C virus of the present disclosure is adjusted to a pH of about 5.7 prior to binding to the cation exchange membrane (e.g., using a poliovirus such as S1 or S2). In some embodiments, an eluate containing an Enterovirus C virus of the present disclosure is adjusted to a pH of about 5.0 prior to binding to the cation exchange membrane (e.g., using a poliovirus such as S3). A variety of devices known in the art are suitable for cation exchange chromatography (optionally including filtration), such as the Mustang® S system (Pall Corporation), which uses a cation exchange membrane with a 0.65 μm pore size. A variety of functional groups are used for cation exchange membranes, including without limitation pendant sulfonic function groups in a cross-linked, polymeric coating. A variety of buffers may be used to bind an eluate containing an Enterovirus C virus of the present disclosure to a cation exchange membrane. Exemplary buffers include, without limitation, citrate and phosphate buffers (additional buffers are described infra). In some embodiments, a buffer used in cation exchange chromatography (e.g., in loading and/or elution) contains polysorbate (e.g., TWEEN®-80 at 0.05%, 0.1%, 0.25%, or 0.5%).

In some embodiments, an eluate containing an Enterovirus C virus of the present disclosure (e.g., poliovirus S1, S2, or S3) is bound to a cation exchange membrane at a pH that ranges from about 4.5 to about 6.0. In some embodiments, the eluate is bound to the cation exchange membrane at a pH that is less than about any of the following pHs: 6.0, 5.5, or 5.0. In some embodiments, the eluate is bound to the cation exchange membrane at a pH that is greater than about any of the following pHs: 4.5, 5.0, or 5.5. That is, the eluate can be bound to the cation exchange membrane at a pH in a range of pHs having an upper limit of 6.0, 5.5, or 5.0 and an independently selected lower limit of 4.5, 5.0, or 5.5; wherein the lower limit is less than the upper limit. In some embodiments, an eluate containing an Enterovirus C virus of the present disclosure is bound to a cation exchange membrane at between about 8 mS/cm and about 10 mS/cm. For example, the eluate may be bound at about 8, about 9, or about 10 mS/cm.

In some embodiments, a bound fraction containing an Enterovirus C virus of the present disclosure (e.g., poliovirus S1, S2, or S3) is eluted from a cation exchange membrane to produce an eluate containing the virus. In some embodiments, cation exchange chromatography of the present disclosure includes a filtration step, e.g., before binding to the membrane, during chromatography, and/or after elution from the membrane. Elution may be gradient or step-wise. As described herein, elution may be effected using a change in pH of the mobile phase or by using a change in ionic strength of the mobile phase (e.g., through addition of a salt). A variety of salts are used for elution, including without limitation sodium chloride, potassium chloride, sodium sulphate, potassium sulphate, ammonium sulphate, sodium acetate, potassium phosphate, calcium chloride, and magnesium chloride. In certain embodiments, the salt is NaCl. For example, in some embodiments, a bound fraction containing an Enterovirus C virus of the present disclosure is eluted from a cation exchange membrane by adding from about 0.20 M to about 0.30 M sodium chloride, e.g., by adding about 200 mM, about 225 mM, about 250 mM, about 275 mM, or about 300 mM sodium chloride. In some embodiments, a bound fraction containing an Enterovirus C virus of the present disclosure is eluted from a cation exchange membrane at between about 20 mS/cm and about 25 mS/cm. e.g., at about 20, about 21, about 22, about 23, about 24, or about 25 mS/cm. A variety of buffers are used for pH elution, including without limitation maleic acid, methyl malonic acid, citric acid, lactic acid, formic acid, succinic acid, acetic acid, MES, phosphate, HEPES, and BICINE. In certain embodiments, the buffer is phosphate or citrate. Suitable pH ranges for elution using each of these buffers are known in the art; generally the pH of the buffer is between the pI of the molecule (e.g., an Enterovirus C virus) and the pKa of the charged groups on the stationary phase. For example, in some embodiments, a bound fraction containing an Enterovirus C virus of the present disclosure is eluted from a cation exchange membrane by adjusting the pH to about 8.0.

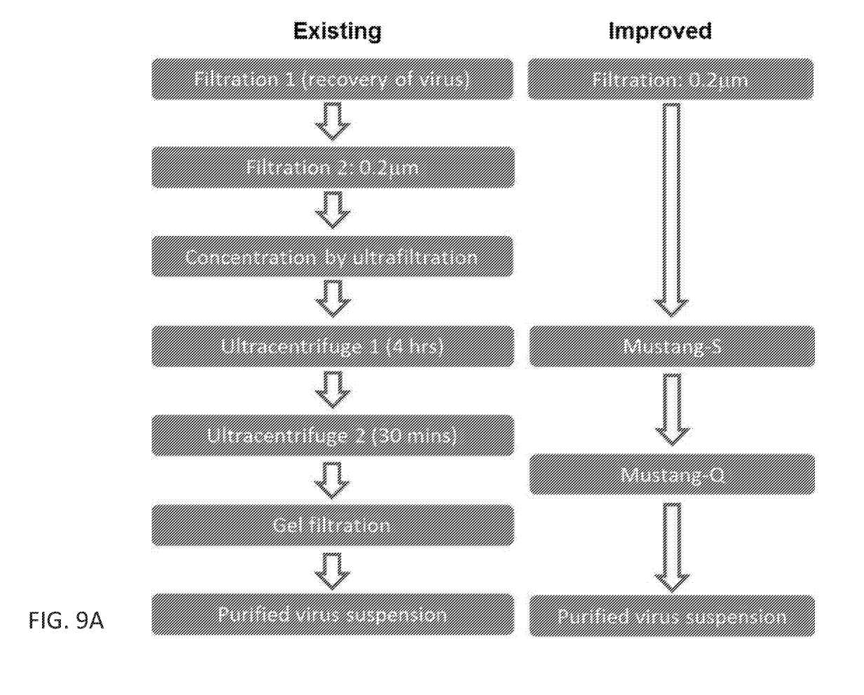

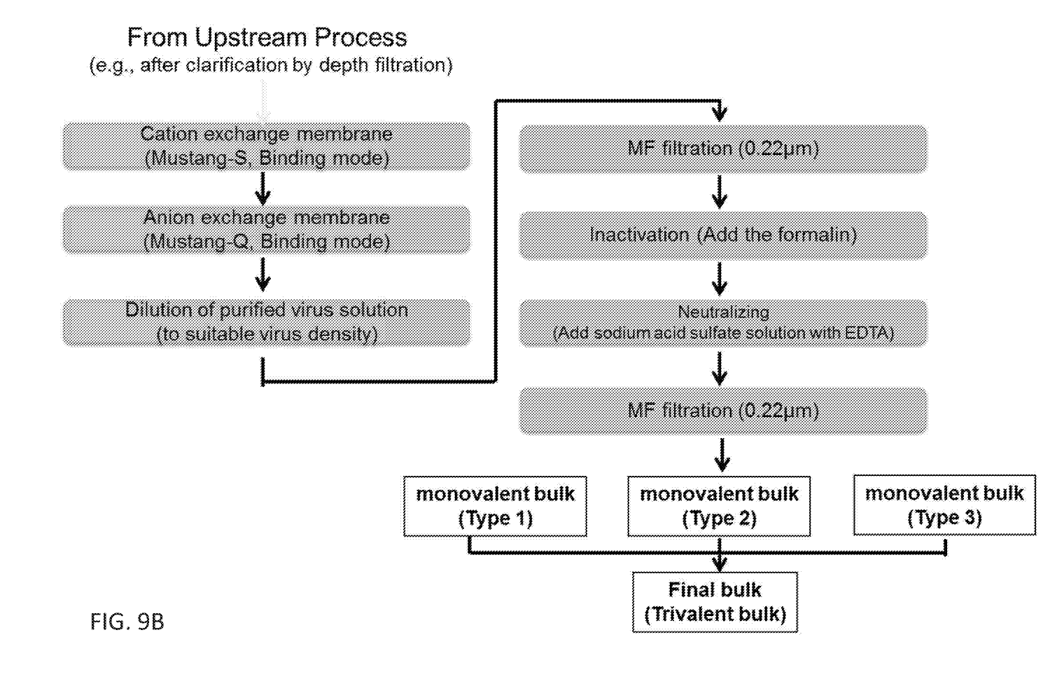

Exemplary cation exchange binding and elution parameters and conditions are described herein. It is contemplated that cation exchange chromatography as described above may employ one or more of the conditions described in Examples 8-10 and 12, and/or in reference to FIGS. 9A-11E, 12A-20B, 21A-23G, and 25-33B, in any combination.

Anion exchange chromatography may be used to purify an Enterovirus C virus of the present disclosure (e.g., poliovirus S1, S2, or S3). As demonstrated herein, the buffers and conditions used for anion exchange chromatography loading (e.g., binding to an anion exchange membrane) and elution greatly impact virus purity and yield. In some embodiments, an eluate containing an Enterovirus C virus of the present disclosure is bound to an anion exchange membrane to produce a bound fraction containing the virus. In some embodiments, the eluate has been subject to depth filtration and/or cation exchange chromatography. In some embodiments, an eluate containing an Enterovirus C virus of the present disclosure is diluted (e.g., using a 2×, 3×, 4×, or 5× dilution factor) prior to anion exchange chromatography. In other embodiments, an eluate containing an Enterovirus C virus of the present disclosure is not diluted prior to anion exchange chromatography. In some embodiments, an eluate containing an Enterovirus C virus of the present disclosure (e.g., using a poliovirus such as S1, S2, or S3) is adjusted to a pH from about 8.0 to about 8.5 prior to binding to the anion exchange membrane. In some embodiments, an eluate containing an Enterovirus C virus of the present disclosure is adjusted to a pH of about 8.0 prior to binding to the anion exchange membrane (e.g., using a poliovirus such as S2). In some embodiments, an eluate containing an Enterovirus C virus of the present disclosure is adjusted to a pH of about 8.5 prior to binding to the anion exchange membrane (e.g., using a poliovirus such as S1 or S3). A variety of devices known in the art are suitable for anion exchange chromatography (optionally including filtration), such as the Mustang® Q system (Pall Corporation), which uses an anion exchange membrane with a 0.8 μm pore size. A variety of functional groups are used for anion exchange membranes, including without limitation pendant quaternary amine functional groups in a cross-linked, polymeric coating. A variety of buffers may be used to bind an eluate containing an Enterovirus C virus of the present disclosure to an anion exchange membrane. Exemplary buffers include, without limitation, phosphate buffer (additional buffers are described infra). In some embodiments, a buffer used in anion exchange chromatography (e.g., in loading and/or elution) contains polysorbate (e.g., TWEEN®-80 at 0.05%, 0.1%, 0.25%, or 0.5%).

In some embodiments, an eluate containing an Enterovirus C virus of the present disclosure (e.g., poliovirus S1, S2, or S3) is bound to an anion exchange membrane at a pH that ranges from about 7.5 to about 8.5. For example, the pH may be adjusted to about 7.5, about 7.6, about 7.7, about 7.8, about 7.9, about 8.0, about 8.1, about 8.2, about 8.3, about 8.4, or about 8.5. In certain embodiments (e.g., using poliovirus S1 or S3), the eluate is bound to anion exchange membrane at a pH of about 8.5. In other embodiments (e.g., using poliovirus S2), the eluate is bound to anion exchange membrane at a pH of about 8.0. In some embodiments, an eluate containing an Enterovirus C virus of the present disclosure is bound to an anion exchange membrane at between about 3 mS/cm.

In some embodiments, a bound fraction containing an Enterovirus C virus of the present disclosure (e.g., poliovirus S1, S2, or S3) is eluted from an anion exchange membrane to produce an eluate containing the virus. In some embodiments, anion exchange chromatography of the present disclosure includes a filtration step, e.g., before binding to the membrane, during chromatography, and/or after elution from the membrane. Elution may be gradient or step-wise. As described herein, elution may be effected using a change in pH of the mobile phase or by using a change in ionic strength of the mobile phase (e.g., through addition of a salt). A variety of salts are used for elution, including without limitation sodium chloride, potassium chloride, sodium sulphate, potassium sulphate, ammonium sulphate, sodium acetate, potassium phosphate, calcium chloride, and magnesium chloride. In certain embodiments, the salt is NaCl. For example, in some embodiments, a bound fraction containing an Enterovirus C virus of the present disclosure is eluted from an anion exchange membrane by adding from about 0.05 M to about 0.10 M sodium chloride. In some embodiments, a bound fraction containing an Enterovirus C virus of the present disclosure is eluted from a cation exchange membrane at between about 5 mS/cm and about 10 mS/cm, e.g., at about 5, about 6, about 7, about 8, about 9, or about 10 mS/cm. A variety of buffers are used for pH elution, including without limitation phosphate, N-methylpiperazine, piperazine, L-histidine, bis-Tris, Bis-Tris propane, triethanolamine, Tris, N-methyl-diethanolamine, diethanolamine, propane 1,3-diamino, ethanolamine, and piperidine. In certain embodiments, the buffer is phosphate or Tris. Suitable pH ranges for elution using each of these buffers are known in the art; generally the pH of the buffer is between the pI of the molecule (e.g., an Enterovirus C virus) and the pKa of the charged groups on the stationary phase.

Exemplary anion exchange binding and elution parameters and conditions are described herein. It is contemplated that anion exchange chromatography as described above may employ one or more of the conditions described in Examples 8-10 and 12, and/or in reference to FIGS. 9A-11E, 12A-20B, 21A-23G, and 25-33B, in any combination.

### Viruses

Certain aspects of the present disclosure relate to producing an Enterovirus C virus. Poliomyelitis in humans is caused by three serotypes of poliovirus (PV1, PV2, and PV3) of the human enterovirus C (HEV-C) group. Human enterovirus C belongs to the Picornaviridae family of non-enveloped, positive-sense RNA viruses, which also includes polioviruses and numerous Coxsackie A virus serotypes (e.g., CAV serotypes 1, 11, 13, 15, 17, 18, 19, 20, 21, 22, and 24) (Brown, B. et al. (2003) J. Virol. 7:8973-84). Accordingly, examples of suitable Enteroviruses C include, without limitation, PV1, PV2, PV3, or any combination, variant, or recombinant thereof. In some embodiments, the Enterovirus C may refer to one or more of the three Sabin strains (e.g., S1, S2, and S3), including recombinants and vaccine-derived variants thereof (see, e.g., Kew O M. Nottay B K. Evolution of the oral poliovaccine strains in humans occur by both mutation and intramolecular recombination. In: Chanock R, Lerner R, editors. Modern approaches to vaccines. N.Y: Cold Spring Harbor Press; 1984. pp. 357-367). Examples of producing all three poliovirus serotypes are provided herein. In certain embodiments, the Enterovirus C virus is a poliovirus strain selected from LSc,2ab (S1); P712,Ch,2ab (S2); Leon, 12_{a1b} (S3); and any combination. These poliovirus strains are known in the art: see, e.g., Toyoda. H. et al. (1984) J. Mol. Biol. 17:561-85.

Accordingly, in some embodiments, an Enterovirus C of the present disclosure (e.g., poliovirus S1, S2, or S3) may be used in any of the vaccines and/or immunogenic compositions disclosed herein. For example, an Enterovirus C of the present disclosure (e.g., poliovirus S1, S2, or S3) may be used to provide one or more antigens or viral strain(s) (e.g., inactivated or live attenuated strain(s)) useful for treating or preventing polio in a subject in need thereof and/or inducing an immune response, such as a protective immune response, against polio in a subject in need thereof.

An antigen of the present disclosure may be derived from an Enterovirus C of the present disclosure (e.g., an Enterovirus C produced and/or purified by the methods described herein, such as poliovirus S1, S2, or S3). An antigen of the present disclosure may be any substance capable of eliciting an immune response. Examples of suitable antigens include, but are not limited to, whole virus, attenuated virus, inactivated virus, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates.

An Enterovirus C of the present disclosure (e.g., poliovirus S1, S2, or S3) may include at least one non-human cell adaptation mutation. Adaptation mutations may be generated by adapting a virus to growth in a particular cell line. For example, a cell may be transfected with a virus and passaged such that the virus replicates and its nucleic acid mutates. Nucleic acid mutations may be point mutations, insertion mutations, or deletion mutations. Nucleic acid mutations may lead to amino acid changes within viral proteins that facilitate growth of the virus in a non-human cell. Adaptation mutations may facilitate phenotypic changes in the virus, including altered plaque size, growth kinetics, temperature sensitivity, drug resistance, virulence, and virus yield in cell culture. These adaptive mutations may be useful in vaccine manufacture by increasing the speed and yield of virus cultured in a cell line. In addition, adaptive mutations may enhance immunogenicity of viral antigens by altering the structure of immunogenic epitopes.

Accordingly, in certain embodiments, an Enterovirus C of the present disclosure (e.g., poliovirus S1, S2, or S3) may include at least one non-human cell adaptation mutation. In certain embodiments, the adaptation mutations are mutations of a viral antigen to a non-human cell. In some embodiments, the non-human cell may be a mammalian cell. Examples of non-human mammalian cells include, without limitation, those described above, such as, Vero cells (from monkey kidneys), MDBK cells, MDCK cells, ATCC CCL34 MDCK (NBL2) cells, MDCK 33016 (deposit number DSM ACC 2219 as described in WO97/37001) cells, BHK21-F cells, HKCC cells, or Chinese hamster ovary cells (CHO cells). In some embodiments, the non-human cell may be a monkey cell. In some embodiments, the monkey cell is from a Vero cell line. Examples of suitable Vero cell lines include, without limitation, WHO Vero 10-87, ATCC CCL-81, Vero 76 (ATCC Accession No. CRL-1587), or Vero C1008 (ATCC Accession No. CRL-1586).

Enteroviruses C such as polioviruses possess linear, positive sense, single-stranded RNA genomes (see, e.g., Brown, B. et al. (2003) J. Virol. 7:8973-84). Each of these viral genomes encodes both structural and nonstructural polypeptides. Structural polypeptides encoded by each of these viruses include, without limitation, VP1, VP2, VP3, and VP4, which together may compose the viral capsid. Non-structural polypeptides encoded by each of these viruses include, without limitation, 2A, 2B, 2C, 3A, 3B, 3C, and 3D, which are involved in, for example, virus replication and virulence.

Accordingly, in certain embodiments, an Enterovirus C of the present disclosure (e.g., poliovirus S1, S2, or S3) may contain at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or more non-human cell adaptation mutations within one or more, two or more, three or more, four or more, five or more, six or more, seven or more, eight or more, nine or more, or ten or more viral antigens, including, without limitation, VP1, VP2, VP3, 2A, 2B, 2C, 3A, 3B, 3C, and 3D. In some embodiments, an Enterovirus A of the present disclosure includes whole, inactivated virus that may contain at least one, at least two, at least three, at least four, at least five, at least six, at least seven, at least eight, at least nine, at least 10, or more non-human cell adaptation mutations within the 5′ or 3′ untranslated region (UTR) of the virus.

In some embodiments, an Enterovirus C of the present disclosure (e.g., poliovirus S1, S2, or S3) may contain at least one, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or more attenuation mutations within one or more, two or more, three or more, four or more, five or more, six or more, seven or more, eight or more, nine or more, or ten or more noncoding regions and/or viral antigens, including, without limitation, 5′ and 3′ noncoding regions, VP1, VP2, VP3, 2A, 2B, 2C, 3A, 3B, 3C, and 3D. For example, attenuation mutations from the Sabin poliovirus strains are known in the art, including without limitation mutations found in the 5′ and 3′ noncoding regions (e.g., IRES mutations), capsid proteins, and so forth (see, e.g., Kawamura, N. et al. (1989) J. Virol. 6:1302-9 and Minor, P. D. (1992) J. Gen. Virol. 7:3065-77).

In some embodiments, an Enterovirus C (e.g., poliovirus S1, S2, or S3) may be used in any of the vaccines and immunogenic compositions of the present disclosure. For example, the Enterovirus C of the present disclosure may be useful for treating or preventing polio in a subject in need thereof and/or inducing an immune response, such as a protective immune response, against polio in a subject in need thereof. In some embodiments, the Enterovirus C may be an inactivated poliovirus or combination of polioviral serotypes useful in a Salk-type inactivated vaccine. In some embodiments, the Enterovirus C may be an attenuated poliovirus or combination of polioviral serotypes useful in a Sabin oral polio vaccine, including recombinants and derivants thereof (see, e.g., Kohara. M. et al. (1988) J. Virol. 6:2828-35 and Shimizu, H. (2016) Vaccine 3:1975-85).

### Production of Antigens

Antigens of the present disclosure for use in vaccines and/or immunogenic compositions including, without limitation, purified viruses, inactivated viruses, attenuated viruses, recombinant viruses, or purified and/or recombinant viral proteins for subunit vaccines to treat or prevent polio and/or induce an immune response, such as a protective immune response, against polio may be produced and/or purified or otherwise isolated by any suitable method known in the art. Antigens of the present disclosure may include, without limitation, whole virus, attenuated virus, inactivated virus, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates produced, derived, purified, and/or otherwise isolated from at least one virus that causes polio. For example, suitable antigens may include, without limitation, structural polypeptides such as VP1, VP2, VP3, and VP4, and non-structural polypeptides, such as 2A, 2B, 2C, 3A, 3B, 3C, and 3D from an Enterovirus C of the present disclosure.

Antigen of the present disclosure may be synthesized chemically or enzymatically, produced recombinantly, isolated from a natural source, or a combination of the foregoing. In certain embodiments, antigens of the present disclosure are produced, purified, isolated, and/or derived from at least one virus of the present disclosure that causes polio, such as S1, S2, and S3 (also known as PV1, PV2, and PV3). Antigens of the present disclosure may be purified, partially purified, or a crude extract. In some embodiments, antigens of the present disclosure are viruses, such as inactivated viruses, produced as described in the above section entitled “Production of Vaccines and Immunogenic Compositions.”

In certain embodiments, one or more antigens of the present disclosure may be produced by culturing a non-human cell. Cell lines suitable for production of the one or more antigens of the present disclosure are preferably of mammalian origin, and include but are not limited to: VERO cells (from monkey kidneys), horse, cow (e.g. MDBK cells), sheep, dog (e.g. MDCK cells from dog kidneys, ATCC CCL34 MDCK (NBL2) or MDCK 33016, deposit number DSM ACC 2219 as described in WO97/37001), cat, and rodent (e.g. hamster cells such as BHK21-F, HKCC cells, or Chinese hamster ovary cells (CHO cells)), and may be obtained from a wide variety of developmental stages, including for example, adult, neonatal, fetal, and embryo. In certain embodiments, the cells are immortalized (e.g. PERC.6 cells, as described in WO01/38362 and WO02/40665, and as deposited under ECACC deposit number 96022940). In preferred embodiments, mammalian cells are utilized, and may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g. epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. WO97/37000 and WO97/37001 describe production of animal cells and cell lines that capable of growth in suspension and in serum free media and are useful in the production of viral antigens. In certain embodiments, the non-human cell is cultured in serum-free media.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. In many embodiments, the antigen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential additional of activated monomeric units to a solid phase bound growing peptide chain.

Well-established recombinant DNA techniques can be employed for production of polypeptides, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

Besides killed and attenuated virus immunogenic compositions, one can use a subunit immunogenic composition or other type of immunogenic composition which presents to the animal the antigenic components of polio. The antigenic component may be a protein, glycoprotein, lipid-conjugated protein or glycoprotein, a modified lipid moiety, or other viral component which, when injected into a human, stimulates an immune response in the human such that the human develops protective immunity against polio. For a subunit immunogenic composition, the virus can be cultured on mammalian cells, as described above. The cell culture can be homogenized and an immunogenic composition can be isolated by passage of the cell culture homogenate over the appropriate column or through the appropriate pore size filter or via centrifugation of the cell culture homogenate.

If the antigenic component is a protein, then one can isolate the nucleic acid which encodes that protein and generate an immunogenic composition that contains that isolated nucleic acid. The nucleic acid encoding the antigenic component can be placed on a plasmid downstream of a signal sequence of a eukaryotic promoter. That plasmid can contain one or more selectable markers and be transfected into an attenuated prokaryotic organism, such as Salmonella spp., Shigella spp., or other suitable bacteria. The bacteria can then be administered to the human so that the human can generate a protective immune response to the antigenic component. Alternatively, the nucleic acid encoding the antigenic component can be placed downstream of a prokaryotic promoter, have one or more selectable markers, and be transfected into an attenuated prokaryotic organism such as Salmonella spp., Shigella spp., or other suitable bacteria. The bacteria can then be administered to the eukaryotic subject for which immune response to the antigen of interest is desired. See, for example, U.S. Pat. No. 6,500,419 to Hone, et al.

For a subunit immunogenic composition, the nucleic acid encoding a proteinaceous antigenic component of a poliovirus can be cloned into a plasmid such as those described in International Patent Application Publication Number WO 00/32047 (Galen) and International Patent Application Publication Number WO 02/083890 (Galen). Then the plasmid can be transfected into bacteria and the bacteria can produce the desired antigenic protein. One can isolate and purify the desired antigenic protein by a variety of methods described in both patent applications.

### Virus Inactivation

In some embodiments, an Enterovirus C virus produced and/or purified by the methods of the present disclosure (e.g., poliovirus S1, S2, or S3) is inactivated. Methods of inactivating or killing viruses to destroy their ability to infect mammalian cells are known in the art. Such methods include both chemical and physical means. Suitable means for inactivating a virus include, without limitation, treatment with an effective amount of one or more agents selected from detergents, formalin (also referred to herein as “formaldehyde”), beta-propiolactone (BPL), binary ethylamine (BEI), acetyl ethyleneimine, heat, electromagnetic radiation, x-ray radiation, gamma radiation, ultraviolet radiation (UV radiation), UV-A radiation, UV-B radiation, UV-C radiation, methylene blue, psoralen, carboxyfullerene (C60) and any combination of any thereof.

Agents for chemical inactivation and methods of chemical inactivation are well-known in the art and described herein. In some embodiments, the Enterovirus A is chemically inactivated with one or more of BPL, formalin, or BEI. In certain embodiments where the Enterovirus A is chemically inactivated with BPL, the virus may contain one or more modifications. In some embodiments, the one or more modifications may include a modified nucleic acid. In some embodiments, the modified nucleic acid is an alkylated nucleic acid. In other embodiments, the one or more modifications may include a modified polypeptide. In some embodiments, the modified polypeptide contains a modified amino acid residue including one or more of a modified cysteine, methionine, histidine, aspartic acid, glutamic acid, tyrosine, lysine, serine, and threonine. In certain embodiments where the Enterovirus C is chemically inactivated with formalin, the virus may contain one or more modifications. In some embodiments, the one or more modifications may include a modified polypeptide. In some embodiments, the one or more modifications may include a cross-linked polypeptide. In some embodiments where the Enterovirus C is chemically inactivated with formalin, the vaccine or immunogenic composition further includes formalin. In certain embodiments of the present disclosure, the Enterovirus C was inactivated with BEI. In certain embodiments where the Enterovirus C was inactivated with BEI, the virus contains one or more modifications. In some embodiments, the one or more modifications includes a modified nucleic acid. In some embodiments, the modified nucleic acid is an alkylated nucleic acid.

In some embodiments where the Enterovirus C (e.g., poliovirus S1, S2, or S3) is chemically inactivated with BEI or BPL, any residual unreacted BEI or BPL may be neutralized (i.e., hydrolyzed) with sodium thiosulfate. Generally, sodium thiosulfate is added in excess. In some embodiments, sodium thiosulfate may be added at a concentration that ranges from about 25 mM to about 100 mM, from, about 25 mM to about 75 mM, or from about 25 mM to about 50 mM. In certain embodiments, sodium thiosulfate may be added at a concentration of about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, or about 40 mM at a ratio of 1 part concentrated sodium thiosulfate to 20 parts of BEI. In some embodiments, the solutions may be mixed using a mixer, such as an in-line static mixer, and subsequently filtered (e.g., clarified). Generally, the pumping of the two solutions through the mixer results in complete mixing and neutralization of BEI by the sodium thiosulfate.

Certain embodiments of the present disclosure relate to a method for inactivating an Enterovirus C (e.g., poliovirus S1, S2, or S3). In some embodiments, the method involves treating the virus preparation with an effective amount of BEI. In certain embodiments, treating with an effective amount of BEI includes, without limitation, treating with BEI in an amount that ranges from about 0.25% v/v to about 3.0% v/v. In certain embodiments, the isolated and treated virus is selected from one or more of PV1, PV2, and PV3. In certain embodiments of the method, the virus preparation is treated with BEI at a temperature that ranges from about 25° C. to about 42° C. In certain embodiments of the method, the virus preparation is treated with BEI for a period of time that ranges from about 1 hour to about 10 hours. In certain embodiments, the method further involves inactivating (i.e., hydrolyzing) unreacted BEI with an effective amount of sodium thiosulfate. In some embodiments, the effective amount of sodium thiosulfate ranges from about 25 mM to about 100 mM, from, about 25 mM to about 75 mM, or from about 25 mM to about 50 mM.

In some embodiments, the method involves treating the virus preparation with an effective amount of beta-propiolactone (BPL); and, optionally, treating the virus preparation with an effective amount of formalin concurrently with or after treating the virus preparation with an effective amount of beta-propiolactone (BPL). Alternatively, in some embodiments, the method involves treating the virus preparation with an effective amount of beta-propiolactone (BPL) for a first period of time; and treating the virus preparation with an effective amount of BPL for a second period of time to completely inactivate the virus preparation. In some embodiments the first and/or second period of time ranges from about 12 hours to about 36 hours. In certain embodiments the first and/or second period of time is about 24 hours. In certain embodiments, treating with an effective amount of BPL includes, without limitation, treating with BPL in an amount that ranges from about 0.05% v/v to about 3.0% v/v, from 0.1% v/v to about 2% v/v, or about 0.1% v/v to about 1% v/v. In certain embodiments, treating with an effective amount of BPL includes, without limitation, treating with 0.05% v/v, 0.06% v/v, 0.07% v/v, 0.08% v/v, 0.09% v/v, 0.1% v/v, 0.2% v/v, 0.3% v/v, 0.4% v/v, 0.5% v/v, 0.6% v/v, 0.7% v/v, 0.8% v/v, 0.9% v/v, or 1% v/v BPL. In certain embodiments of the method, the virus preparation is treated with BET at a temperature that ranges from about 2° C. to about 8° C. In certain embodiments, the method involves heating the virus preparation at a temperature of 37° C. for a period of time sufficient to hydrolyze the BPL. In certain embodiments, the period of time ranges from about 1 hour to about 6 hours. Alternatively, in some embodiments, the method further involves inactivating (i.e., hydrolyzing) unreacted BPL with an effective amount of sodium thiosulfate. In some embodiments, the effective amount of sodium thiosulfate ranges from about 25 mM to about 100 mM, from, about 25 mM to about 75 mM, or from about 25 mM to about 50 mM.

In some embodiments, the method involves treating the virus preparation with an effective amount of formalin; and purifying the virus preparation from the formalin. In certain embodiments, treating with an effective amount of formalin includes, without limitation, treating with formalin in an amount that ranges from about 0.05% v/v to about 3.0% v/v, from 0.1% v/v to about 2% v/v, or about 0.1% v/v to about 1% v/v. In certain embodiments, the virus preparation is purified to a high degree from the formalin in an amount that is about 70%, about 75%, about 80%, about 85%, about 90%, about 95%, about 96%, about 97%, about 98%, about 99%, or more. Methods for inactivating polioviruses using formalin are well known in the art; see, e.g., Wilton, T. et al. (2014) J. Virol. 8:11955-64.

Formulations of Vaccines and/or Immunogenic Compositions

Further aspects of the present disclosure relate to compositions, immunogenic compositions, and/or vaccines containing a virus (e.g., an Enterovirus C of the present disclosure, such as poliovirus S1, S2, or S3) produced by the methods of the present disclosure. Such compositions, vaccines, and/or immunogenic compositions may be useful for treating or preventing polio in a subject in need thereof and/or inducing an immune response, such as a protective immune response, against polio in a subject in need thereof.

Typically, vaccines and/or immunogenic compositions of the present disclosure are prepared as injectables either as liquid solutions or suspensions: solid forms suitable for solution in, or suspension in, liquid prior to injection may also be prepared. Such preparations may also be emulsified or produced as a dry powder. The active immunogenic ingredient is often mixed with excipients which are pharmaceutically acceptable and compatible with the active ingredient. Suitable excipients are, for example, water, saline, dextrose, sucrose, glycerol, ethanol, or the like, and combinations thereof. In addition, if desired, the vaccine or immunogenic composition may contain auxiliary substances such as wetting or emulsifying agents, pH buffering agents, or adjuvants which enhance the effectiveness of the vaccine or immunogenic composition.

Vaccines or immunogenic compositions may be conventionally administered parenterally, by injection, for example, either subcutaneously, transcutaneously, intradermally, subdermally or intramuscularly. Additional formulations which are suitable for other modes of administration include suppositories and, in some cases, oral, peroral, intranasal, buccal, sublingual, intraperitoneal, intravaginal, anal and intracranial formulations. Oral and injected polio vaccines are well known in the art and have been used for more than 50 years. For suppositories, traditional binders and carriers may include, for example, polyalkalene glycols or triglycerides; such suppositories may be formed from mixtures containing the active ingredient in the range of 0.5% to 10%, or even 1-2%. In certain embodiments, a low melting wax, such as a mixture of fatty acid glycerides or cocoa butter is first melted and the hand, foot, and mouth disease vaccine or immunogenic composition antigens described herein are dispersed homogeneously, for example, by stirring. The molten homogeneous mixture is then poured into conveniently sized molds, allowed to cool, and to solidify.

Formulations suitable for intranasal delivery include liquids (e.g., aqueous solution for administration as an aerosol or nasal drops) and dry powders (e.g. for rapid deposition within the nasal passage). Formulations include such normally employed excipients as, for example, pharmaceutical grades of mannitol, lactose, sucrose, trehalose, xylitol, and chitosan. Mucosadhesive agents such as chitosan can be used in either liquid or powder formulations to delay mucociliary clearance of intranasally-administered formulations. Sugars such as mannitol and sucrose can be used as stability agents in liquid formulations and as stability, bulking, or powder flow and size agents in dry powder formulations. In addition, adjuvants such as monophosphoryl lipid A (MLA), or derivatives thereof, or CpG oligonucleotides can be used in both liquid and dry powder formulations as an immunostimulatory adjuvant.

Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations include such normally employed excipients as, for example, pharmaceutical grades of mannitol, lactose, starch, magnesium stearate, sodium saccharine, cellulose, magnesium carbonate, and the like. Other hand, foot, and mouth disease vaccine and immunogenic compositions may take the form of solutions, suspensions, pills, sustained release formulations or powders and contain 10-95% of active ingredient, or 25-70%. For oral formulations, cholera toxin is an interesting formulation partner (and also a possible conjugation partner).

The polio vaccines and/or immunogenic compositions when formulated for vaginal administration may be in the form of pessaries, tampons, creams, gels, pastes, foams or sprays. Any of the foregoing formulations may contain agents in addition to polio vaccine and immunogenic composition antigens, such as carriers, known in the art to be appropriate.

In some embodiments, the polio vaccines and/or immunogenic compositions of the present disclosure may be formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., intradermal, topical application, intravenous, intramuscular, etc.

The vaccines and/or immunogenic compositions of the present disclosure may be administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with an exemplary range from about 0.1 μg to 10 μg (even though higher amounts in the 1-10 mg range are contemplated), such as in the range from about 0.1 μg to 5 μg, or even in the range from 0.6 μg to 3 μg or in the range from about 1 μg to 3 μg, or even in the range of 0.1 μg to 1 μg. In certain embodiments, the dosage can be about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 2.1 μg, about 2.2 μg, about 2.3 μg, about 2.4 μg, about 2.5 μg, about 2.6 μg, about 2.7 μg, about 2.8 μg, about 2.9 μg, or about 3 μg per dose. In certain embodiments, vaccines and/or immunogenic compositions of the present disclosure may be administered in an amount of 1 μg per dose.

In some embodiments, dosage is based on a number of units, such as D-antigen units for a poliovirus vaccine. In some embodiments, a vaccine and/or immunogenic composition of the present disclosure contains dosages of poliovirus strains S1, S2, and S3. For example, suitable dosages for a vaccine and/or immunogenic composition of the present disclosure may include without limitation ratios of 1.5:5:50, 0.7:25:25, or 3:10:100 of S1:S2:S3 (e.g., ratios based on DU of each strain).

Suitable regimens for initial administration and booster shots are also variable but are typified by an initial administration followed by subsequent inoculations or other administrations. Administration may vary for adults, infants, and those with complicating indications, such as renal impairment. Dosing regiments suitable for administering oral polio vaccines (OPVs) and inactivated polio vaccines (IPVs) are known in the art. For example, in adults and children, the Orimune polio vaccine may be administered in a single 0.5 mL oral dose, in some embodiments followed by a second dose 8 weeks later, and a third dose 8-12 months after the second dose. In infants, a first 0.5 mL oral dose may be administered at 6-12 weeks of age, followed by a second 0.5 mL oral dose 8 weeks after the first dose, and a third 0.5 mL oral dose administered between 6 and 18 months of age. According to the World Health Organization, OPV vaccination regimens may include 3 OPV doses plus 1 IPV dose, with dosing initiated from the age of 6 weeks and a minimum interval of 4 weeks between OPV doses. If a single IPV dose is used, it is preferably given from 14 weeks and may be co-administered with an OPV dose. For IPV administration alone, a primary series of three IPV doses may be administered beginning at 2 months of age, and a booster dose may be administered after an interval of at least 6 months.

In some embodiments, an Enterovirus C of the present disclosure (e.g., poliovirus S1, S2, or S3) may be produced for use in a combination vaccine. For example, Pediarix® (GlaxoSmithKline) is a combination inactivated polio, DTaP, and hepatitis B vaccine; Kinrix® (GlaxoSmithKline) is a combination inactivated polio and DTaP vaccine; Pentacel® (Sanofi Pasteur) is a combination inactivated polio, DTaP, and influenza vaccine; and Quadracel® (Sanofi Pasteur) is a combination inactivated polio and DTaP vaccine.

The manner of application may be varied widely. Any of the conventional methods for administration of a vaccine or immunogenic composition are applicable. These include oral application on a solid physiologically acceptable base or in a physiologically acceptable dispersion, parenterally, by injection or the like. The dosage of the vaccine or immunogenic composition will depend on the route of administration and will vary according to the age of the person to be vaccinated and the formulation of the antigen.

Delivery agents that improve mucoadhesion can also be used to improve delivery and immunogenicity especially for intranasal, oral or lung based delivery formulations. One such compound, chitosan, the N-deacetylated form of chitin, is used in many pharmaceutical formulations. It is an attractive mucoadhesive agent for intranasal vaccine delivery due to its ability to delay mucociliary clearance and allow more time for mucosal antigen uptake and processing. In addition, it can transiently open tight junctions which may enhance transepithelial transport of antigen to the NALT. In a recent human trial, a trivalent inactivated influenza vaccine administered intranasally with chitosan but without any additional adjuvant yielded seroconversion and HI titers that were only marginally lower than those obtained following intramuscular inoculation.

Vaccines and/or immunogenic compositions of the present disclosure are pharmaceutically acceptable. They may include components in addition to the antigen and adjuvant e.g. they will typically include one or more pharmaceutical carrier(s) and/or excipient(s). A thorough discussion of such components is available in Gennaro (2000) Remington: The Science and Practice of Pharmacy. 20th edition, ISBN: 0683306472.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Vaccines and/or immunogenic compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a vaccine or immunogenic composition will generally be between 5.0 and 8.1, and more typically between 6.0 and 8.0 e.g. 6.5 and 7.5, or between 7.0 and 7.8. A manufacturing process of the present disclosure may therefore include a step of adjusting the pH of the bulk vaccine prior to packaging.

The vaccine or immunogenic composition is preferably sterile. It is preferably non pyrogenic e.g. containing <1 EU (endotoxin unit, a standard measure) per dose, and preferably <0.1 EU per dose. It is preferably gluten free.

In certain embodiments, the vaccines and/or immunogenic compositions of the present disclosure may include a detergent in an effective concentration. In some embodiments, an effective amount of detergent may include without limitation, about 0.00005% v/v to about 5% v/v or about 0.0001% v/v to about 1% v/v. In certain embodiments, an effective amount of detergent is about 0.001% v/v, about 0.002% v/v, about 0.003% v/v, about 0.004% v/v, about 0.005% v/v, about 0.006% v/v, about 0.007% v/v, about 0.008% v/v, about 0.009% v/v, or about 0.01% v/v. Without wishing to be bound by theory, detergents help maintain the vaccines and/or immunogenic compositions of the present disclosure in solution and helps to prevent the vaccines and/or immunogenic compositions from aggregating.

Suitable detergents include, for example, polyoxyethylene sorbitan ester surfactant (known as ‘Tweens’), octoxynol (such as octoxynol-9 (TRITON™ X-100) or t octylphenoxypolyethoxyethanol), cetyl trimethyl ammonium bromide (‘CTAB’), and sodium deoxycholate, particularly for a split or surface antigen vaccine. The detergent may be present only at trace amounts. Other residual components in trace amounts could be antibiotics (e.g. neomycin, kanamycin, polymyxin B). In some embodiments, the detergent contains polysorbate. In some embodiments, the effective concentration of detergent includes ranges from about 0.00005% v/v to about 5% v/v.

The vaccines and/or immunogenic compositions are preferably stored at between 2° C. and 8° C. They should not be frozen. They should ideally be kept out of direct light. The antigen and emulsion will typically be in admixture, although they may initially be presented in the form of a kit of separate components for extemporaneous admixing. Vaccines and/or immunogenic compositions will generally be in aqueous form when administered to a subject.

### Adjuvants

Compositions, immunogenic compositions, and/or vaccines of the present disclosure may be used in combination with one or more adjuvants. Such adjuvanted vaccines and/or immunogenic compositions of the present disclosure may be useful for treating or preventing polio in a subject in need thereof and/or inducing an immune response, such as a protective immune response, against polio in a subject in need thereof. Several types of adjuvants, including aluminum adjuvants, calcium phosphate, oil emulsions, chitosan, vitamin D, oligonucleotides, stearyl or octadecyl tyrosine, and liposomes have been used and their effectiveness characterized in IPVs and OPVs (see, e.g., Hawken J. and Troy S. B. (2012) Vaccine 3:6971-9).

Various methods of achieving an adjuvant effect for vaccines are known and may be used in conjunction with the polio vaccines and/or immunogenic compositions disclosed herein. General principles and methods are detailed in “The Theory and Practical Application of Adjuvants”, 1995. Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York. ISBN 0-306-45283-9.

In some embodiments, a polio vaccine or immunogenic composition includes the antigens and an adjuvant. Antigens may be in a mixture with at least one adjuvant, at a weight-based ratio of from about 1:1 to about 10¹⁰:1 antigen:adjuvant, e.g., from about 1:1 to about 10:1, from about 10:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 antigen:adjuvant. One of skill in the art can readily determine the appropriate ratio through information regarding the adjuvant and routine experimentation to determine optimal ratios.

Exemplary adjuvants may include, but are not limited to, aluminum salts, toll-like receptor (TLR) agonists, monophosphoryl lipid A (MLA), MLA derivatives, synthetic lipid A, lipid A mimetics or analogs, cytokines, saponins, muramyl dipeptide (MDP) derivatives. CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, Complete Freund's Adjuvant (CFA), and Incomplete Freund's Adjuvant (IFA). In some embodiments, the adjuvant is MLA or derivatives thereof.

In some embodiments, the adjuvant is an aluminum salt. In some embodiments, the adjuvant includes at least one of alum, aluminum phosphate, aluminum hydroxide, potassium aluminum sulfate, and Alhydrogel 85. In some embodiments, aluminum salt adjuvants of the present disclosure have been found to increase adsorption of the antigens of the Enterovirus C virus (e.g., poliovirus S1, S2, or S3) vaccines and/or immunogenic compositions of the present disclosure. Accordingly, in some embodiments, at least 75%, at least 80%, at least 85%, at least 90%, at least 91%, at least 92%, at least 93%, at least 94%, at least 95%, at least 96%, at least 97%, at least 98%, at least 99%, or 100% of the antigen is adsorbed to the aluminum salt adjuvant.

Monophosphoryl Lipid A (MLA), a non-toxic derivative of lipid A from Salmonella, is a potent TLR-4 agonist that has been developed as a vaccine adjuvant (Evans et al. 2003). In pre-clinical murine studies intranasal MLA has been shown to enhance secretory, as well as systemic, humoral responses (Baldridge et al. 2000; Yang et al. 2002). It has also been proven to be safe and effective as a vaccine adjuvant in clinical studies of greater than 120,000 patients (Baldrick et al., 2002; 2004). MLA stimulates the induction of innate immunity through the TLR-4 receptor and is thus capable of eliciting nonspecific immune responses against a wide range of infectious pathogens, including both gram negative and gram positive bacteria, viruses, and parasites (Baldrick et al. 2004; Persing et al. 2002). Inclusion of MLA in intranasal formulations should provide rapid induction of innate responses, eliciting nonspecific immune responses from viral challenge while enhancing the specific responses generated by the antigenic components of the vaccine.

Accordingly, in one embodiment, the present disclosure provides a composition comprising monophosphoryl lipid A (MLA), 3 De-O-acylated monophosphoryl lipid A (3D-MLA), or a derivative thereof as an enhancer of adaptive and innate immunity. Chemically 3D-MLA is a mixture of 3 De-O-acylated monophosphoryl lipid A with 4, 5 or 6 acylated chains. A preferred form of 3 De-O-acylated monophosphoryl lipid A is disclosed in European Patent 0 689 454 B 1 (SmithKline Beecham Biologicals SA). In another embodiment, the present disclosure provides a composition comprising synthetic lipid A, lipid A mimetics or analogs, such as BioMira's PET Lipid A, or synthetic derivatives designed to function like TLR-4 agonists.

Additional exemplary adjuvants include, without limitation, polypeptide adjuvants that may be readily added to the antigens described herein by co-expression with the polypeptide components or fusion with the polypeptide components to produce chimeric polypeptides. Bacterial flagellin, the major protein constituent of flagella, is an adjuvant which has received increasing attention as an adjuvant protein because of its recognition by the innate immune system by the toll-like receptor TLR5 (65). Flagellin signaling through TLR5 has effects on both innate and adaptive immune functions by inducing DC maturation and migration as well as activation of macrophages, neutrophils, and intestinal epithelial cells resulting in production of proinflammatory mediators (66-72).

TLR5 recognizes a conserved structure within flagellin monomers that is unique to this protein and is required for flagellar function, precluding its mutation in response to immunological pressure (73). The receptor is sensitive to a 100 fM concentration but does not recognize intact filaments. Flagellar disassembly into monomers is required for binding and stimulation.

As an adjuvant, flagellin has potent activity for induction of protective responses for heterologous antigens administered either parenterally or intranasally and adjuvant effects for DNA vaccines have also been reported. A Th2 bias is observed when flagellin is employed which would be appropriate for a respiratory virus such as influenza but no evidence for IgE induction in mice or monkeys has been observed. In addition, no local or systemic inflammatory responses have been reported following intranasal or systemic administration in monkeys. The Th2 character of responses elicited following use of flagellin is somewhat surprising since flagellin signals through TLR5 in a MyD88-dependent manner and all other MyD88-dependent signals through TLRs have been shown to result in a Th1 bias. Importantly, pre-existing antibodies to flagellin have no appreciable effect on adjuvant efficacy making it attractive as a multi-use adjuvant.

Cytokines, colony-stimulating factors (e.g., GM-CSF, CSF, and the like); tumor necrosis factor; interleukin-2, -7, -12, interferons and other like growth factors, may also be used as adjuvants as they may be readily included in the polio vaccines or immunogenic compositions by admixing or fusion with the polypeptide component.

In some embodiments, the polio vaccine and immunogenic compositions disclosed herein may include other adjuvants that act through a Toll-like receptor such as a nucleic acid TLR9 ligand comprising a 5′-TCG-3′ sequence; an imidazoquinoline TLR7 ligand; a substituted guanine TLR7/8 ligand; other TLR7 ligands such as Loxoribine, 7-deazadeoxyguanosine, 7-thia-8-oxodeoxyguanosine, Imiquimod (R-837), and Resiquimod (R-848).

Certain adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminum adjuvants; DNA adjuvants; MLA; and an encapsulating adjuvant.

Additional examples of adjuvants include agents such as aluminum salts such as hydroxide or phosphate (alum), commonly used as 0.05 to 0.1 percent solution in buffered saline (see, e.g., Nicklas (1992) Res. Immunol. 14:489-493), admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA may also be used.

DDA (dimethyldioctadecylammonium bromide) is an interesting candidate for an adjuvant, but also Freund's complete and incomplete adjuvants as well as quillaja saponins such as QuilA and QS21 are interesting. Further possibilities include poly[di(earboxylatophenoxy)phosphazene (PCPP) derivatives of lipopolysaccharides such as monophosphoryl lipid A (MLA), muramyl dipeptide (MDP) and threonyl muramyl dipeptide (tMDP). The lipopolysaccharide based adjuvants may also be used for producing a predominantly Th1-type response including, for example, a combination of monophosphoryl lipid A, such as 3-de-O-acylated monophosphoryl lipid A, together with an aluminum salt.

Liposome formulations are also known to confer adjuvant effects, and therefore liposome adjuvants may be used in conjunction with the hand, foot, and mouth disease vaccines and/or immunogenic compositions.

Immunostimulating complex matrix type (ISCOM® matrix) adjuvants may also be used with the hand, foot, and mouth disease vaccine antigens and immunogenic compositions, especially since it has been shown that this type of adjuvants are capable of up-regulating MHC Class II expression by APCs. An ISCOM matrix consists of (optionally fractionated) saponins (triterpenoids) from Quillaja saponaria, cholesterol, and phospholipid. When admixed with the immunogenic protein such as the polio vaccine or immunogenic composition antigens, the resulting particulate formulation is what is known as an ISCOM particle where the saponin may constitute 60-70% w/w, the cholesterol and phospholipid 10-15% w/w, and the protein 10-15% w/w. Details relating to composition and use of immunostimulating complexes can for example be found in the above-mentioned text-books dealing with adjuvants, but also Morein B et al., 1995, Clin. Immunother. 3: 461-475 as well as Barr T G and Mitchell G F, 1996, Immunol. and Cell Biol. 74: 8-25 provide useful instructions for the preparation of complete immunostimulating complexes.

The saponins, whether or not in the form of iscoms, that may be used in the adjuvant combinations with the hand, foot, and mouth disease vaccine antigens and immunogenic compositions disclosed herein include those derived from the bark of Quillaja Saponaria Molina, termed Quil A, and fractions thereof, described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B. Exemplary fractions of Quil A are QS21, QS7, and QS17.

β-Escin is another hemolytic saponins for use in the adjuvant compositions of the hand, foot, and mouth disease vaccines and/or immunogenic compositions. Escin is described in the Merck index (12th ed: entry 3737) as a mixture of saponins occurring in the seed of the horse chestnut tree. Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 August; 44(8): 1454-1464)). β-escin is also known as aescin.

Another hemolytic saponin for use in the hand, foot, and mouth disease vaccines and/or immunogenic compositions is Digitonin. Digitonin is described in the Merck index (12^{th} Edition, entry 3204) as a saponin, being derived from the seeds of Digitalis purpurea and purified according to the procedure described Gisvold et al., J. Am. Pharm. Assoc., 1934, 23, 664; and Ruhenstroth-Bauer, Physiol. Chem., 1955, 301, 621. Its use is described as being a clinical reagent for cholesterol determination.

Another interesting possibility of achieving adjuvant effect is to employ the technique described in Gosselin et al., 1992. In brief, the presentation of a relevant antigen such as an antigen in a polio vaccine and/or immunogenic composition of the present disclosure can be enhanced by conjugating the antigen to antibodies (or antigen binding antibody fragments) against the F_{C} receptors on monocytes/macrophages. Especially conjugates between antigen and anti-F_{C}RI have been demonstrated to enhance immunogenicity for the purposes of vaccination. The antibody may be conjugated to the hand, foot, and mouth disease vaccine or immunogenic composition antigens after generation or as a part of the generation including by expressing as a fusion to any one of the polypeptide components of the polio vaccine and immunogenic composition antigens.

Other possibilities involve the use of the targeting and immune modulating substances (i.e. cytokines). In addition, synthetic inducers of cytokines such as poly I:C may also be used.

Suitable mycobacterial derivatives may be selected from the group consisting of muramyl dipeptide, complete Freund's adjuvant, RIBI. (Ribi ImmunoChem Research Inc., Hamilton. Mont.) and a diester of trehalose such as TDM and TDE.

Examples of suitable immune targeting adjuvants include CD40 ligand and CD40 antibodies or specifically binding fragments thereof (cf. the discussion above), mannose, a Fab fragment, and CTLA-4.

Examples of suitable polymer adjuvants include a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York. N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12-15, 1998, Seascape Resort. Aptos, Calif.”

Oligonucleotides may be used as adjuvants in conjunction with the polio vaccine and immunogenic composition antigens and may contain two or more dinucleotide CpG motifs separated by at least three or more or even at least six or more nucleotides. CpG-containing oligonucleotides (in which the CpG dinucleotide is unmethylated) induce a predominantly Th1 response. Such oligonucleotides are well known and are described, for example, in WO 96/02555, WO 99/33488 and U.S. Pat. Nos. 6,008,200 and 5,856,462.

Such oligonucleotide adjuvants may be deoxynucleotides. In certain embodiments, the nucleotide backbone in the oligonucleotide is phosphorodithioate, or a phosphorothioate bond, although phosphodiester and other nucleotide backbones such as PNA including oligonucleotides with mixed backbone linkages may also be used. Methods for producing phosphorothioate oligonucleotides or phosphorodithioate are described in U.S. Pat. Nos. 5,666,153, 5,278,302 and WO95/26204.

Exemplary oligonucleotides have the following sequences. The sequences may contain phosphorothioate modified nucleotide backbones:

(SEQ ID NO: 1) OLIGO 1:
TCC ATG ACG TTC CTG ACG TT (CpG 1826);
(SEQ ID NO: 2) OLIGO 2:
TCT CCC AGC GTG CGC CAT (CpG 1758);
(SEQ ID NO: 3) OLIGO 3:
ACC GAT GAC GTC GCC GGT GAC GGC ACC ACG;
(SEQ ID NO: 4) OLIGO 4:
TCG TCG TTT TGT CGT TTT GTC GTT (CpG 2006);
and
(SEQ ID NO: 5) OLIGO 5:
TCC ATG ACG TTC CTG ATG CT (CpG 1668)

Alternative CpG oligonucleotides include the above sequences with inconsequential deletions or additions thereto. The CpG oligonucleotides as adjuvants may be synthesized by any method known in the art (e.g., EP 468520). For example, such oligonucleotides may be synthesized utilizing an automated synthesizer. Such oligonucleotide adjuvants may be between 10-50 bases in length. Another adjuvant system involves the combination of a CpG-containing oligonucleotide and a saponin derivative particularly the combination of CpG and QS21 is disclosed in WO 00/09159.

Many single or multiphase emulsion systems have been described. One of skill in the art may readily adapt such emulsion systems for use with polio vaccines and immunogenic composition antigens so that the emulsion does not disrupt the antigen's structure. Oil in water emulsion adjuvants per se have been suggested to be useful as adjuvant compositions (EPO 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use with the hand, foot, and mouth disease vaccines and/or immunogenic compositions described herein may be natural or synthetic, and may be mineral or organic. Examples of mineral and organic oils will be readily apparent to one skilled in the art.

In order for any oil in water composition to be suitable for human administration, the oil phase of the emulsion system may include a metabolizable oil. The meaning of the term metabolizable oil is well known in the art. Metabolizable can be defined as “being capable of being transformed by metabolism” (Dorland's Illustrated Medical Dictionary. W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils may also be used and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and may be used with the hand, foot, and mouth disease vaccine and immunogenic composition antigens. Squalene is a metabolizable oil virtue of the fact that it is an intermediate in the biosynthesis of cholesterol (Merck index, 10th Edition, entry no. 8619).

Exemplary oil emulsions are oil in water emulsions, and in particular squalene in water emulsions.

In addition, the oil emulsion adjuvants for use with the polio vaccine and immunogenic composition antigens may include an antioxidant, such as the oil α-tocopherol (vitamin E, EP 0 382 271 B1).

WO 95/17210 and WO 99/11241 disclose emulsion adjuvants based on squalene, α-tocopherol, and TWEEN®-80, optionally formulated with the immunostimulants QS21 and/or 3D-MLA. WO 99/12565 discloses an improvement to these squalene emulsions with the addition of a sterol into the oil phase. Additionally, a triglyceride, such as tricaprylin (C27H50O6), may be added to the oil phase in order to stabilize the emulsion (WO 98/56414).

The size of the oil droplets found within the stable oil in water emulsion may be less than 1 micron, may be in the range of substantially 30-600 nm, substantially around 30-500 nm in diameter, or substantially 150-500 nm in diameter, and in particular about 150 nm in diameter as measured by photon correlation spectroscopy. In this regard, 80% of the oil droplets by number may be within these ranges, more than 90% or more than 95% of the oil droplets by number are within the defined size ranges. The amounts of the components present in oil emulsions are conventionally in the range of from 2 to 10% oil, such as squalene; and when present, from 2 to 10% alpha tocopherol; and from 0.3 to 3% surfactant, such as polyoxyethylene sorbitan monooleate. The ratio of oil:alpha tocopherol may be equal or less than 1 as this provides a more stable emulsion. SPAN 85 (TM) may also be present at a level of about 1%. In some cases it may be advantageous that the polio vaccines and/or immunogenic compositions disclosed herein will further contain a stabilizer.

The method of producing oil in water emulsions is well known to one skilled in the art. Commonly, the method includes the step of mixing the oil phase with a surfactant such as a PBS/TWEEN80® solution, followed by homogenization using a homogenizer, it would be clear to one skilled in the art that a method comprising passing the mixture twice through a syringe needle would be suitable for homogenizing small volumes of liquid. Equally, the emulsification process in microfluidizer (M 11 OS microfluidics machine, maximum of 50 passes, for a period of 2 minutes at maximum pressure input of 6 bar (output pressure of about 850 bar)) could be adapted by one skilled in the art to produce smaller or larger volumes of emulsion. This adaptation could be achieved by routine experimentation comprising the measurement of the resultant emulsion until a preparation was achieved with oil droplets of the required diameter.

Alternatively the polio vaccines and/or immunogenic compositions may be combined with vaccine vehicles composed of chitosan (as described above) or other polycationic polymers, polylactide and polylactide-coglycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM.

Additional illustrative adjuvants for use in the polio vaccines and/or immunogenic compositions as described herein include SAF (Chiron, Calif., United States), MF-59 (Chiron, see, e.g., Granoff et al. (1997) Infect Immun. 65 (5): 1710-1715), the SBAS series of adjuvants (e.g., SB-AS2 (an oil-in-water emulsion containing MLA and QS21); SBAS-4 (adjuvant system containing alum and MLA), available from SmithKline Beecham, Rixensart, Belgium), Detox (Enhanzyn®) (GlaxoSmithKline), RC-512, RC-522, RC-527, RC-529, RC-544, and RC-560 (GlaxoSmithKline) and other aminoalkyl glucosaminide 4-phosphates (AGPs), such as those described in pending U.S. patent application Ser. Nos. 08/853,826 and 09/074,720.

Other examples of adjuvants include, but are not limited to, Hunter's TiterMax® adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 14:553-557); alum (e.g., aluminum hydroxide, aluminum phosphate) emulsion based formulations including mineral oil, non-mineral oil, water-in-oil or oil-in-water emulsions, such as the Seppic ISA series of Montamide adjuvants (e.g., ISA-51, ISA-57, ISA-720, ISA-151, etc.; Seppic, Paris, France); and PROVAX® (IDEC Pharmaceuticals); OM-174 (a glucosamine disaccharide related to lipid A); Leishmania elongation factor; non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

Other exemplary adjuvants include adjuvant molecules of the general formula:

HO(CH₂CH₂O)ₙ-A-R, (I)

where, n is 1-50, A is a bond or —C(O)—, R is C₁₋₅₀ alkyl or Phenyl C₁₋₅₀ alkyl.

One embodiment consists of a vaccine formulation comprising a polyoxyethylene ether of general formula (I), where n is between 1 and 50, 4-24, or 9; the R component is C₁₋₅₀, C₄-C₂₀ alkyl, or C₁₂ alkyl, and A is a bond. The concentration of the polyoxyethylene ethers should be in the range 0.1-20%, from 0.1-10%, or in the range 0.1-1%. Exemplary polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, an adjuvant combination may include the CpG as described above.

Further examples of suitable pharmaceutically acceptable excipients for use with the polio vaccines and/or immunogenic compositions disclosed herein include water, phosphate buffered saline, isotonic buffer solutions.

In some embodiments, a vaccine formulation of the present disclosure includes Sabin inactivated polio vaccine (sIPV) against S1, S2, and S3 (PV1, PV2, and PV3) with alum at 0.5 mg/dose. In other embodiments, a vaccine formulation of the present disclosure includes Sabin inactivated polio vaccine (sIPV) against type I, II, and III sIPV with alum at 0.067 mg/mL. In other embodiments, a vaccine formulation of the present disclosure includes Sabin inactivated polio vaccine (sIPV) against type I, II, and III sIPV plus diphtheria, tetanus, and pertussis vaccines with alum at 0.133 mg/mL.

Further aspects of the present disclosure relate to methods for using vaccines and/or or immunogenic compositions of the present disclosure containing one or more antigens from at least one virus that causes polio to treat or prevent polio in a subject in need thereof and/or to induce an immune response to polio in a subject in need thereof. In some embodiments, the present disclosure relates to methods for treating or preventing polio in a subject in need thereof by administering to the subject a therapeutically effective amount of a vaccine and/or or immunogenic composition of the present disclosure containing one or more antigens from at least one virus that causes polio. In some embodiments, the present disclosure relates to methods for inducing an immune response to polio in a subject in need thereof by administering to the subject a therapeutically effective amount of a vaccine and/or immunogenic composition of the present disclosure containing one or more antigens from at least one virus that causes polio. Any of the methods of the present disclosure may use inactivated or live, attenuated polivirus(es).

In some embodiments, the protective immune response includes an immune response against one or more of PV1, PV2, and PV3.

In some embodiments, the administering step includes one or more administrations. Administration can be by a single dose schedule or a multiple dose (prime-boost) schedule. In a multiple dose schedule the various doses may be given by the same or different routes e.g. a parenteral prime and mucosal boost, a mucosal prime and parenteral boost, etc. Typically they will be given by the same route. Multiple doses will typically be administered at least 1 week apart (e.g. about 2 weeks, about 3 weeks, about 4 weeks, about 6 weeks, about 8 weeks, about 12 weeks, about 16 weeks, etc.). Giving two doses separated by from 25-30 days (e.g. 28 days) is particularly useful. As described above, exemplary dosing regimens for polio vaccination are known in the art.

The methods of the present disclosure include administration of a therapeutically effective amount or an immunogenic amount of the vaccines and/or immunogenic compositions of the present disclosure. A therapeutically effective amount or an immunogenic amount may be an amount of the vaccines and/or immunogenic compositions of the present disclosure that will induce a protective immunological response in the uninfected, infected or unexposed subject to which it is administered. Such a response will generally result in the development in the subject of a secretory, cellular and/or antibody-mediated immune response to the vaccine. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell.

Preferably, the therapeutically effective amount or immunogenic amount is sufficient to bring about treatment or prevention of disease symptoms. The exact amount necessary will vary depending on the subject being treated; the age and general condition of the subject to be treated; the capacity of the subject's immune system to synthesize antibodies; the degree of protection desired; the severity of the condition being treated; the particular hand, foot, and mouth disease antigen polypeptide selected and its mode of administration, among other factors. An appropriate therapeutically effective amount or immunogenic amount can be readily determined by one of skill in the art. A therapeutically effective amount or immunogenic amount will fall in a relatively broad range that can be determined through routine trials.

### ENUMERATED EMBODIMENTS

The following enumerated embodiments are representative of some aspects of the present disclosure.

1. A method for producing an Enterovirus C virus, comprising:

(a) culturing a cell in a first cell culture medium;

(b) inoculating the cell with the Enterovirus C virus in a second cell culture medium under conditions in which the Enterovirus C virus infects the cell and the infected cell produces the Enterovirus C virus; and

(c) harvesting the Enterovirus C virus produced by the cell,

wherein a surfactant is added to the second cell culture medium during inoculation of the cell with the Enterovirus C virus or from approximately one hour to approximately four hours prior to step (c).
2. The method of embodiment 1, wherein the yield of Enterovirus C virus harvested in step (c) is increased, as compared to a yield of Enterovirus C virus harvested in the absence of the surfactant.
3. The method of embodiment 1 or embodiment 2, wherein the surfactant is a polysorbate.
4. The method of embodiment 1 or embodiment 2, wherein the surfactant is a polyethylene glycol-based surfactant.
5. The method of any one of embodiments 1-4, wherein the Enterovirus C virus is a poliovirus serotype selected from the group consisting of S1, S2, and S3.
6. The method of any one of embodiments 1-5, wherein the cell is cultured in step (a) in a liquid culture.
7. The method of any one of embodiments 1-5, wherein the cell is an adherent cell, and the cell is cultured in step (a) on a microcarrier.
8. The method of any one of embodiments 1-5, wherein the cell is an adherent cell, and the cell is cultured in step (a) in a fixed bed comprising a matrix.
9. The method of any one of embodiments 1-5, wherein the cell is cultured in step (a) in a bioreactor.
10. The method of embodiment 8, wherein the cell is inoculated with the Enterovirus C virus at an MOI of between about 0.01 and about 0.0009.
11. The method of embodiment 8 or embodiment 10, wherein between about 120,000 cells/cm² and about 300,000 cells/cm² are inoculated.
12. The method of embodiment 8 or embodiment 10, wherein between about 4,000 cells/cm² and about 16,000 cells/cm² are inoculated.
13. The method of embodiment 12, wherein about 5.000×) cells/cm² are inoculated.
14. The method of any one of embodiments 8, or 10-13, wherein the cell is cultured during steps (a) and/or (b) at a volume/surface ratio of about 0.1 mL/cm² to about 0.3 mL/cm².
15. The method of any one of embodiments 8 and 10-14, wherein step (b) further comprises culturing the inoculated cell under conditions in which the Enterovirus C virus infects the cell and the infected cell produces the Enterovirus C virus.
16. The method of any one of embodiments 8 and 10-15, wherein the cell is inoculated at a pH that ranges from about 6.8 to about 7.4.
17. The method of any one of embodiments 8 and 10-16, wherein no additional glucose is added to the second cell culture medium.
18. The method of any one of embodiments 8 and 10-17, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

19. A method for producing an Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus, wherein the cell is inoculated with the Enterovirus C virus at an MOI of between about 0.01 and about 0.0009; and

(c) harvesting the Enterovirus C virus produced by the cell.

20. The method of embodiment 19, wherein the Enterovirus C virus is a poliovirus serotype selected from the group consisting of S1, S2, and S3.
21. The method of embodiment 19 or embodiment 20, wherein polysorbate is added to the second cell culture medium during inoculation of the cell with the Enterovirus C virus or from approximately one hour to approximately four hours prior to step (c).
22. The method of any one of embodiments 19-21, wherein between about 120,000 cells/cm² and about 300,000 cells/cm² are inoculated.
23. The method of any one of embodiments 19-21, wherein between about 4,000 cells/cm² and about 12,000 cells/cm² are inoculated.
24. The method of embodiment 23, wherein about 5,000 cells/cm² are inoculated.
25. The method of any one of embodiments 19-24, wherein the cell is cultured during steps (a) and/or (b) at a volume/surface ratio of about 0.1 mL/cm² to about 0.3 mL/cm².
26. The method of any one of embodiments 19-25, wherein step (b) further comprises culturing the inoculated cell under conditions in which the Enterovirus C virus infects the cell and the infected cell produces the Enterovirus C virus.
27. The method of any one of embodiments 19-26, wherein the cell is inoculated at a pH that ranges from about 6.8 to about 7.4.
28. The method of any one of embodiments 19-27, wherein no additional glucose is added to the second cell culture medium.
29. The method of any one of embodiments 19-28, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

30. A method for producing an Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus, wherein between about 100,000 cells/cm² and about 320,000 cells/cm² are inoculated; and

(c) harvesting the Enterovirus C virus produced by the cell.

31. The method of embodiment 30, wherein between about 120,000 cells/cm² and about 300,000 cells/cm² are inoculated.
32. The method of embodiment 30, wherein between about 120,000 cells/cm² and about 250,000 cells/cm² are inoculated
33. The method of embodiment 30, wherein about 200,000 cells/cm² are inoculated.
34. The method of embodiment 30, wherein the Enterovirus C virus is a poliovirus serotype selected from the group consisting of S1, S2, and S3.
35. The method of any one of embodiments 30-34, wherein polysorbate is added to the second cell culture medium during inoculation of the cell with the Enterovirus C virus or from approximately one hour to approximately four hours prior to step (c).
36. The method of any one of embodiments 30-35, wherein the cell is inoculated with the Enterovirus C virus at an MOI of between about 0.01 and about 0.0009.
37. The method of any one of embodiments 30-36, wherein the cell is cultured during steps (a) and/or (b) at a volume/surface ratio of about 0.1 mL/cm² to about 0.3 mL/cm².
38. The method of any one of embodiments 30-37, wherein step (b) further comprises culturing the inoculated cell under conditions in which the Enterovirus C virus infects the cell and the infected cell produces the Enterovirus C virus.
39. The method of any one of embodiments 30-38, wherein the cell is inoculated at a pH that ranges from about 6.8 to about 7.4.
40. The method of any one of embodiments 30-39, wherein no additional glucose is added to the second cell culture medium.
41. The method of any one of embodiments 30-40, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

42. A method for producing an Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus; and

(c) harvesting the Enterovirus C virus produced by the cell,

wherein the cell is cultured during steps (a), and/or (b) at a volume/surface ratio of about 0.1 mL/cm² to about 0.3 mL/cm².
43. The method of embodiment 42, wherein the Enterovirus C virus is a poliovirus serotype selected from the group consisting of S1, S2, and S3.
44. The method of embodiment 42 or embodiment 43, wherein polysorbate is added to the second cell culture medium during inoculation of the cell with the Enterovirus C virus or from approximately one hour to approximately four hours prior to step (c).
45. The method of any one of embodiments 42-44, wherein the cell is inoculated with the Enterovirus C virus at an MOI of between about 0.01 and about 0.0009.
46. The method of any one of embodiments 42-45, wherein between about 120.000×) cells/cm² and about 300,000 cells/cm² are inoculated.
47. The method of any one of embodiments 42-45, wherein between about 4,000 cells/cm² and about 16,000 cells/cm² are inoculated.
48. The method of embodiment 47, wherein about 5,000 cells/cm² are inoculated.
49. The method of any one of embodiments 42-48, wherein step (b) further comprises culturing the inoculated cell under conditions in which the Enterovirus C virus infects the cell and the infected cell produces the Enterovirus C virus.
50. The method of any one of embodiments 42-49, wherein the cell is inoculated at a pH that ranges from about 6.8 to about 7.4.
51. The method of any one of embodiments 42-50, wherein no additional glucose is added to the second cell culture medium.
52. The method of any one of embodiments 42-51, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

53. A method for producing an Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus, wherein the cell is inoculated at a pH that ranges from about 6.8 to about 7.4; and

(c) harvesting the Enterovirus C virus produced by the cell.

54. The method of embodiment 53, wherein the Enterovirus C virus is a poliovirus serotype selected from the group consisting of S1, S2, and S3.
55. The method of embodiment 53 or embodiment 54, wherein polysorbate is added to the second cell culture medium during inoculation of the cell with the Enterovirus C virus or from approximately one hour to approximately four hours prior to step (c).
56. The method of any one of embodiments 53-55, wherein the cell is inoculated with the Enterovirus C virus at an MOI of between about 0.01 and about 0.0009.
57. The method of any one of claims 53-56, wherein between about 120,000 cells/cm² and about 300,000 cells/cm² are inoculated.
58. The method of any one of embodiments 53-56, wherein between about 4,000 cells/cm² and about 16.000×) cells/cm² are inoculated.
59. The method of embodiment 58, wherein about 5,000 cells/cm² are inoculated.
60. The method of any one of embodiments 53-59, wherein the cell is cultured during steps (a) and/or (b) at a volume/surface ratio of about 0.1 mL/cm² to about 0.3 mL/cm².
61. The method of any one of embodiments 53-60, wherein step (b) further comprises culturing the inoculated cell under conditions in which the Enterovirus C virus infects the cell and the infected cell produces the Enterovirus C virus.
62. The method of any one of embodiments 53-61, wherein no additional glucose is added to the second cell culture medium.
63. The method of any one of embodiments 53-62, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

64. A method for producing an Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus, wherein no additional glucose is added to the second cell culture medium and/or glucose is depleted from the second culture medium; and

(c) harvesting the Enterovirus C virus produced by the cell.

65. The method of embodiment 64, wherein the Enterovirus C virus is a poliovirus serotype selected from the group consisting of S1, S2, and S3.
66. The method of embodiment 64 or embodiment 65, wherein polysorbate is added to the second cell culture medium during inoculation of the cell with the Enterovirus C virus or from approximately one hour to approximately four hours prior to step (c).
67. The method of any one of embodiments 64-66, wherein the cell is inoculated with the Enterovirus C virus at an MOI of between about 0.01 and about 0.0009.
68. The method of any one of embodiments 64-67, wherein between about 120.000 cells/cm² and about 300,000 cells/cm² are inoculated.
69. The method of any one of embodiments 64-67, wherein between about 4,000 cells/cm² and about 16,000 cells/cm² are inoculated.
70. The method of embodiment 69, wherein about 5,000 cells/cm² are inoculated.
71. The method of any one of embodiments 64-70, wherein the cell is cultured during steps (a) and/or (b) at a volume/surface ratio of about 0.1 mL/cm² to about 0.3 mL/cm².
72. The method of any one of embodiments 64-71, wherein step (b) further comprises culturing the inoculated cell under conditions in which the Enterovirus C virus infects the cell and the infected cell produces the Enterovirus C virus.
73. The method of any one of embodiments 64-72, wherein the cell is inoculated at a pH that ranges from about 6.8 to about 7.4.
74. The method of any one of embodiments 64-73, further comprising, after step (c):

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

75. A method for producing a purified Enterovirus C virus, comprising:

(a) culturing an adherent cell in a fixed bed comprising a matrix, wherein the cell is cultured in a first cell culture medium;

(b) inoculating the cell with the Enterovirus C virus in a second cell culture medium under conditions in which the Enterovirus C virus infects the cell and the infected cell produces the Enterovirus C virus;

(c) harvesting the Enterovirus C virus produced by the cell;

(d) passing the harvested Enterovirus C virus produced by the cell through a depth filter to produce a first eluate, wherein the first eluate comprises the Enterovirus C virus;

(e) binding the first eluate to a cation exchange membrane to produce a first bound fraction, wherein the first bound fraction comprises the Enterovirus C virus;

(f) eluting the first bound fraction from the cation exchange membrane to produce a second eluate, wherein the second eluate comprises the Enterovirus C virus;

(g) binding the second eluate to an anion exchange membrane to produce a second bound fraction, wherein the second bound fraction comprises the Enterovirus C virus; and

(h) eluting the second bound fraction from the anion exchange membrane to produce a purified Enterovirus C virus.

76. The method of embodiment 75, wherein the Enterovirus C virus is a poliovirus serotype selected from the group consisting of S1, S2, and S3.
77. The method of any one of embodiments 18, 29, 41, 52, 63, and 74-76, wherein the depth filter has a pore size of between about 0.2 μm and about 3 μm.
78. The method of any one of embodiments 18, 29, 41, 52, 63, and 74-77, wherein before step (e) the pH of the first eluate is adjusted to a pH value of about 5.7.
79. The method of embodiment 78, wherein the Enterovirus C virus is a poliovirus serotype selected from the group consisting of S1 and S2.
80. The method of any one of embodiments 18, 29, 41, 52, 63, and 74-76, wherein before step (e) the pH of the first eluate is adjusted to a pH value of about 5.0, and wherein the Enterovirus C virus is poliovirus S3.
81. The method of any one of embodiments 18, 29, 41, 52, 63, and 74-80, wherein a phosphate buffer is used to bind the first eluate to the cation exchange membrane.
82. The method of any one of embodiments 18, 29, 41, 52, 63, and 74-81, wherein a buffer comprising polysorbate is used to bind the first eluate to the cation exchange membrane.
83. The method of any one of embodiments 18, 29, 41, 52, 63, and 74-82, wherein the first eluate is bound to the cation exchange membrane at a pH that ranges from about 4.5 to about 6.0.
84. The method of any one of embodiments 18, 29, 41, 52, 63, and 74-83, wherein the first eluate is bound to the cation exchange membrane at between about 7 mS/cm and about 10 mS/cm.
85. The method of any one of embodiments 18, 29, 41, 52, 63, and 74-84, wherein the first bound fraction is eluted by adjusting the pH to about 8.0.
86. The method of any one of embodiments 18, 29, 41, 52, 63, and 74-85, wherein the first bound fraction is eluted by adding from about 0.20 M to about 0.30 M sodium chloride.
87. The method of any one of embodiments 18, 29, 41, 52, 63, and 74-86, wherein the first bound fraction is eluted at between about 20 mS/cm and about 25 mS/cm.
88. The method of any one of embodiments 18, 29, 41, 52, 63, and 74-87, wherein before step (g) the pH of the second eluate is adjusted to a pH value of about 8.0 to about 8.5.
89. The method of embodiment 88, wherein the Enterovirus C virus is a poliovirus serotype selected from the group consisting of S1, S2, and S3, and wherein before step (g) the pH of the second eluate is adjusted to a pH value of about 8.0 to about 8.5.
90. The method of embodiment 88, wherein the Enterovirus C virus is a poliovirus serotype selected from the group consisting of S1 and S3, and wherein before step (g) the pH of the second eluate is adjusted to a pH value of about 8.5.
91. The method of embodiment 88, wherein the Enterovirus C virus is a poliovirus serotype selected from the group consisting of S2 and S3, and wherein before step (g) the pH of the second eluate is adjusted to a pH value of about 8.0.
92. The method of any one of embodiments 18, 29, 41, 52, 63, and 74-91, wherein a phosphate buffer or Tris buffer is used to bind the second eluate to the anion exchange membrane.
93. The method of any one of embodiments 18, 29, 41, 52, 63, and 74-92, wherein a buffer comprising polysorbate is used to bind the second eluate to the anion exchange membrane.
94. The method of any one of embodiments 18, 29, 41, 52, 63, and 74-93, wherein the second eluate is bound to the anion exchange membrane at a pH that ranges from about 7.5 to about 8.5.
95. The method of any one of embodiments 18, 29, 41, 52, 63, and 74-94, wherein the second eluate is bound to the anion exchange membrane at about 3 mS/cm.
96. The method of any one of embodiments 18, 29, 41, 52, 63, and 74-95, wherein the second bound fraction is eluted by adding from about 0.05 M to about 0.10 M sodium chloride.
97. The method of any one of embodiments 18, 29, 41, 52, 63, and 74-96, wherein the second bound fraction is eluted at between about 5 mS/cm and about 10 mS/cm.
98. The method of any one of embodiments 75-97, wherein polysorbate is added to the second cell culture medium during inoculation of the cell with the Enterovirus C virus or from approximately one hour to approximately four hours prior to step (c).
99. The method of any one of embodiments 75-98, wherein the cell is inoculated with the Enterovirus C virus at an MOI of between about 0.01 and about 0.0009.
100. The method of any one of embodiments 75-99, wherein between about 120,000 cells/cm² and about 300,000 cells/cm² are inoculated.
101. The method of any one of embodiments 75-99, wherein between about 4,000 cells/cm² and about 16,000 cells/cm² are inoculated.
102. The method of embodiment 101, wherein about 5,000 cells/cm² are inoculated.
103. The method of any one of embodiments 75-102, wherein the cell is cultured during steps (a) and/or (b) at a volume/surface ratio of about 0.1 mL/cm² to about 0.3 mL/cm².
104. The method of any one of embodiments 75-103, wherein step (b) further comprises culturing the inoculated cell under conditions in which the Enterovirus C virus infects the cell and the infected cell produces the Enterovirus C virus, and wherein step (c) comprises lysing the cell to harvest the Enterovirus C virus produced by the cell.
105. The method of any one of embodiments 75-104, wherein the cell is inoculated at a pH that ranges from about 6.8 to about 7.4.
106. The method of any one of embodiments 75-105, wherein no additional glucose is added to the second cell culture medium and/or glucose is depleted from the second culture medium.
107. The method of any one of embodiments 1-106, wherein the cell is a mammalian cell.
108. The method of embodiment 107, wherein the cell is a Vero cell.
109. The method of embodiment 108, wherein the Vero cell line is selected from the group consisting of WHO Vero 10-87, ATCC CCL-81, Vero 76 (ATCC Accession No. CRL-1587), and Vero C1008 (ATCC Accession No. CRL-1586).
110. The method of any one of embodiments 1-109, wherein between about 4,000 cells/cm² and about 16,000 cells/cm² are cultured in step (a).
111. The method of embodiment 109, wherein about 5,000 cells/cm² are cultured in step (a).
112. The method of any one of embodiments 1-111, wherein the first cell culture medium and the second cell culture medium are different.
113. The method of embodiment 112, further comprising, between steps (a) and (b), removing the first cell culture medium and rinsing the cell with the second culture medium.
114. The method of any one of embodiments 1-113, wherein the density of oxygen (DO) in the first cell culture medium during step (a) is maintained above about 50%.
115. The method of any one of embodiments 1-114, wherein density of oxygen (DO) in the second cell culture medium during step (b) is maintained above about 50%.
116. The method of any one of embodiments 8-115, wherein the fixed bed has a bed height of about 2 cm.
117. The method of any one of embodiments 8-116, wherein the fixed bed has a bed height of about 10 cm.
118. The method of any one of embodiments 8-117, wherein the matrix is a fiber matrix.
119. The method of embodiment 118, wherein the fiber matrix is a carbon matrix.
120. The method of embodiment 118 or claim 119, wherein the fiber matrix has a porosity between about 60% and 99%.
121. The method of embodiment 120, wherein the porosity is between about 80% and about 90%.
122. The method of any one of embodiments 118-121, wherein the fiber matrix has a surface area accessible to the cell of between about 150 cm²/cm³ and about 1000 cm²/cm³.
123. The method of any one of embodiments 118-121, wherein the fiber matrix has a surface area accessible to the cell of between about 10 cm²/cm³ and about 150 cm²/cm³.
124. The method of embodiment 123, wherein the fiber matrix has a surface area accessible to the cell of about 120 cm²/cm³.
125. The method of any one of embodiments 1-124, wherein at least 5.0×10⁷ TCID50/mL of the Enterovirus C virus is harvested in step (d).
126. The method of any one of embodiments 1-125, wherein the Enterovirus C virus is a poliovirus strain selected from the group consisting of LSc,2ab; P712,Ch,2ab; Leon,12_{a1b}; and any combination thereof.
127. The method of any one of embodiments 1-126, further comprising inactivating the Enterovirus C with one or more of beta-propiolactone (BPL), formalin, or binary ethylenimine (BEI).
128. An Enterovirus C virus produced by the method of any one of embodiments 1-127.
129. The Enterovirus C virus of embodiment 128, wherein the virus comprises one or more antigens.

The present disclosure will be more fully understood by reference to the following Examples. They should not, however, be construed as limiting any aspect or scope of the present disclosure in any way.

### EXAMPLES

### Example 1: Effect of Cell Density at Infection (CDI) on Poliovirus Production Using ICELLis NANO®

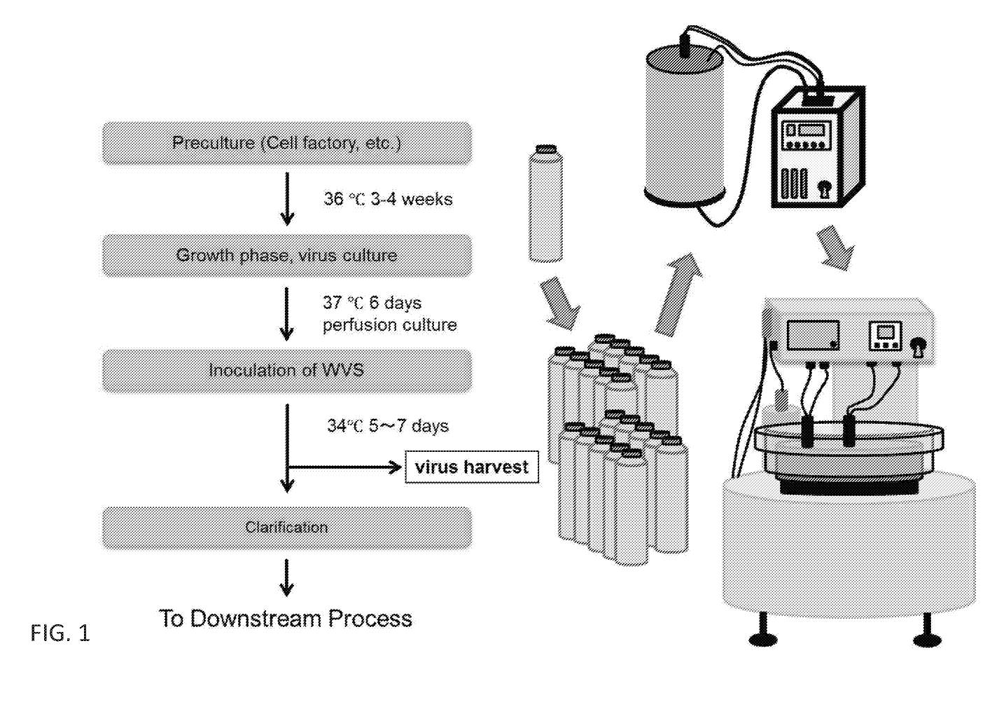

Fixed-bed bioreactor systems (e.g., iCELLis® Bioreactors from Pall® Life Sciences, Port Washington, N.Y., such as the Nano and 500/100 bioreactors) may allow for improved efficiency of host cell growth and virus production on an industrial scale useful for vaccine production (e.g., the Sabin inactivated polio vaccine or sIPV). Upstream process steps for virus production using these systems are illustrated in FIG. 1. These systems are thought to enable increased yield and decreased production costs. For example, a fixed-bed bioreactor system may require only 38 days of upstream processing, as compared to 57 days for a microcarrier culture-based system. However, implementing a fixed-bed bioreactor system for virus production requires identifying optimal conditions for a number of manufacturing parameters before industrial scale production is cost-effective or even possible. These parameters include various aspects of upstream process steps, such as pre-culture growth and culture conditions, cell density at seeding and infection, multiplicity of infection (MOI), culture/growth phase pH, temperature (e.g., at pre-culture, growth phase, and/or virus culturing), and culture duration (e.g., at pre-culture, growth phase, and/or virus culturing).

Cell density at infection (CDI) was examined for potential impact on productivity in the iCELLis® NANO system.

Methods

### Virus and Cell Stocks

S2 poliovirus and Vero cells originally from ATCC® CCL-81™ were used.

### Measurement of D-Antigen

A standard ELISA assay was used to assay D-antigen.

### Glucose/Lactate Measurement

Lactate was measured using a Scout™ lactometer. Glucose was measured using an ACCU-CHEK Aviva Plus system.

### Vero Resuscitation

Vero cell resuscitation was performed. A 1-ml vial of Vero cells was quickly thawed in a 37° C. pre-warmed water bath, and mixed with 10 mL of growth medium at room temperature in a 15 mL centrifuging tube. An aliquot (1.0 ml) was taken for cell count and viability. The remainder was mixed with 20 mL of growth medium pre-warmed at 37° C. in a 50 mL centrifuging tube, and 10 mL of the cell suspension was mixed with 77 mL of pre-warmed growth medium 37° C. in each of 3 T-175 flasks. These flasks were placed at 37° C. in a CO2 (5%) incubator. The next day, the spent medium was discarded and replaced with fresh medium (87 ml), and the culture was continued.

### Cell Culture and Infection

Unless otherwise specified, the following culture conditions were used. These conditions are based at least in part on the improvements to various parameters described infra.

For pre-culture, Vero cells were cultured in DMEM supplemented with 5% FBS in a volume of 0.5 mL/cm² in vented T-175 flasks (Sarstedt AG & Co., Nurembrecht Germany). Cells were passaged for 4 days at a seeding density of 15,000 cells/cm².

For rinsing, cells were rinsed in 800 mL M199 media at 34.0° C. with no DO or pH regulation. Cells were agitated at 530 rpm, and the rinsing time was 15 minutes.

For growth phase in iCELLis NANO®, cells were grown at 37° C. at a seeding density of 5,000 cells/cm² in 1600 mL of DMEM supplemented with 5% FBS and 1 g/L fructose. The total microcarrier area was 5300 cm² (which corresponds to a V/S of 0.3 mL/cm² and allowable densities of up to 0.2×10⁶ cells/cm². For higher densities, 0.5 mL/cm² was used.). Agitation rate was 430 rpm. The pH was 7.15, and the DO was >50%.

For infection phase, cell density was 0.125-0.2×10⁶ cells/cm². Cells were infected at a MOI of 0.002 in 1600 mL of M 199 medium supplemented with 3.5 g/L glucose and 0.05% TWEEN®-80 at 34.0° C. with DO regulated at >50% and pH regulated at 7.40. Cells were agitated at 430 rpm and incubated for 6 days.

### Downstream Processing

Parameters used for downstream acidification, cation exchange chromatography, and anion exchange chromatography steps are described infra.

Results

In order to identify an optimal density at infection for productivity, cell cultures were grown in 5 iCELLis® NANO fixed-bed bioreactors, each infected at a different cell density between 0.1 and 0.5×10⁶ cells/cm². Productivity was measured as D-antigen content.

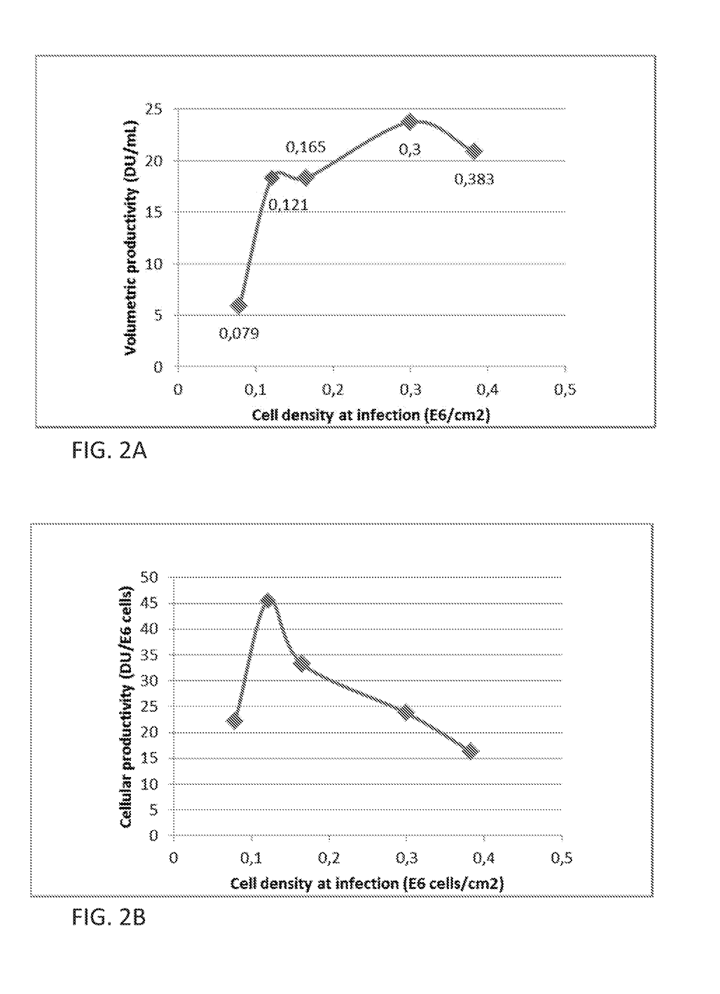

The results were plotted to show productivity as a function of volume (i.e., DU/mL; FIG. 2A) or as a function of cell number (i.e., DU/10⁶ cells; FIG. 2B). These data indicate that near-maximal productivity was achieved at only 0.12×10⁶ cells/cm², with stable productivity near maximum achieved at a large range of CDIs between 0.12-0.35×10⁶ cells/cm². Advantageously, these results indicate that near-maximal productivity may be achieved at lower cell densities, thereby lessening cell culture medium consumption. For example, targeting a CDI of 0.12-0.2×10⁶ cells/cm² is achievable with a volume/surface ratio (V/S) of 0.3 mL/cm², whereas higher CDIs require higher V/S, and therefore greater media consumption. Stated another way, using higher CDIs offer no significant improvement in productivity while resulting in higher media consumption.

### Example 2: Effect of Multiplicity of Infection (MOI) on Polovirus Production Using ICELLis NANO®

Next, the effect of MOT on productivity was examined.

Results

Cells were cultured and infected with virus as described in Example 1. Volumetric productivity was measured as described in reference to FIG. 2A supra.

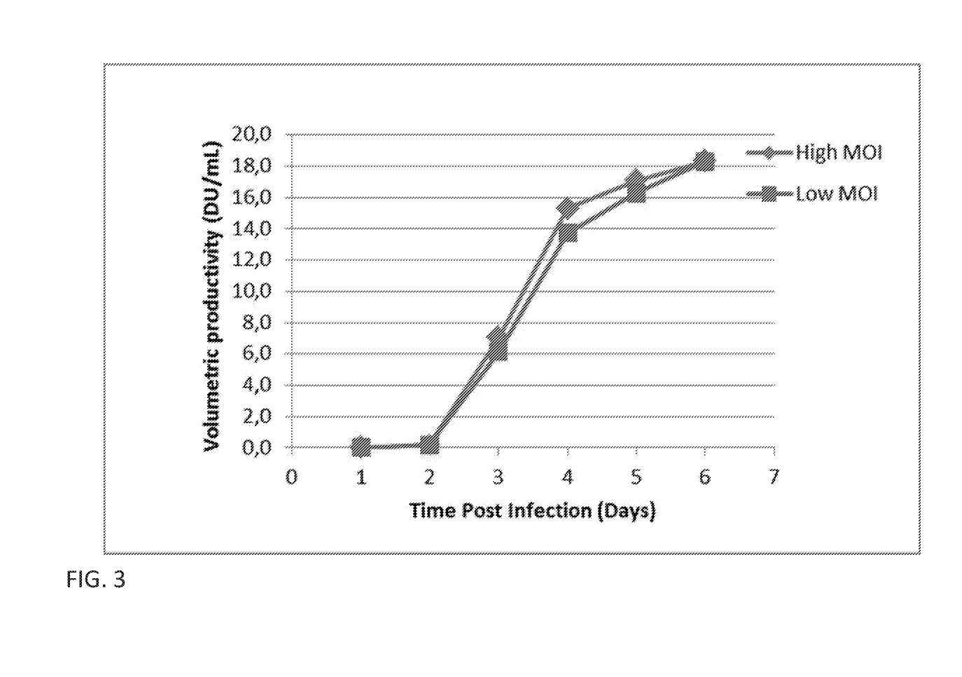

Cells were grown in 2 iCELLis® NANO fixed-bed bioreactors and infected with virus at an MOI of either 0.01 (“high MOI”) or 0.002 (“low MOI”). Both were infected at similar CDIs (0.12×10⁶ cells/cm² for low MOI and 0.16×10⁶ cells/cm² for high MOI). As shown in FIG. 3, both MOIs yielded similar productivity, as assayed by volumetric productivity (DU/mL). Thus, smaller virus MOIs may be used while still attaining maximal productivity and virus release kinetics. An MOI of 0.002 was therefore used for subsequent experiments.

### Example 3: Cell Culture Production Using ICELLis NANO® as Compared to T-Flasks

Compared to the use of a standard iCELLis® NANO system (i.e., before the improvements described herein), productivity is two- to three-fold higher when cells are grown in conventional tissue culture flasks. One difference between the iCELLis® NANO system and tissue culture flasks is that the NANO uses a dynamic environment with circulating media, whereas flasks are a static environment. The effect of iCELLis® NANO conditions on virus stability was therefore tested. Another difference is in the regulation of pH and DO, so the effects of these on productivity in an iCELLis® NANO were also tested.

Results

Cells were cultured and infected with virus as described in Example 1. Volumetric productivity was measured as described in reference to FIG. 2A supra.

The final harvest from a single iCELLis® NANO bioreactor was divided per the following: 200 mL of culture was incubated in a CS-1 flask at 34° C. in a CO₂ incubator for 5 days (“CS control”), and 1200 mL of culture was recirculated in the iCELLis® NANO for 5 days at 34° C. stirring, with 50% DO, at pH7.4, with 30 mL/min air (“iCELLis NANO”).

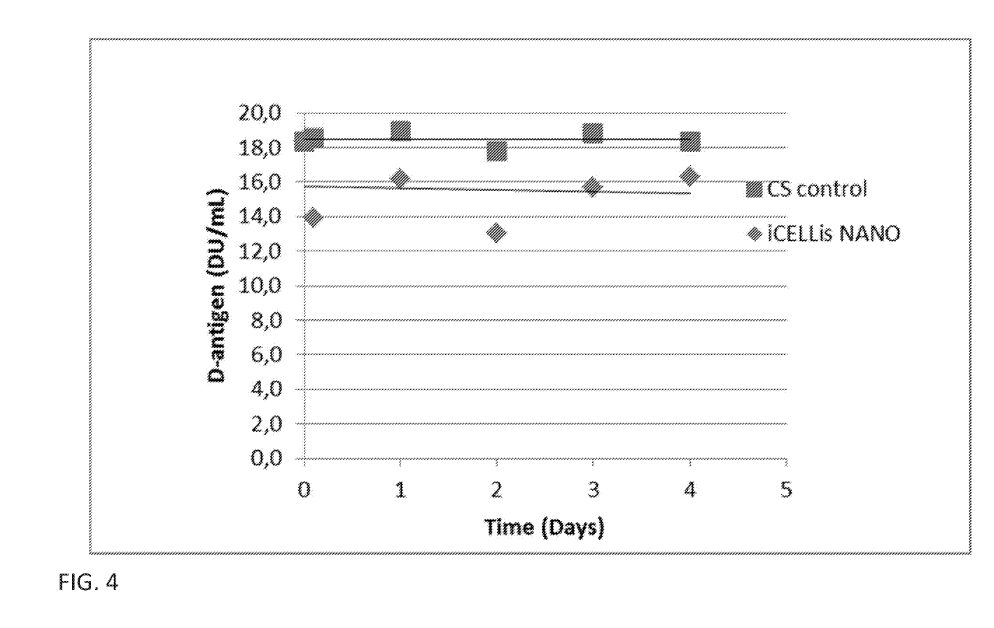

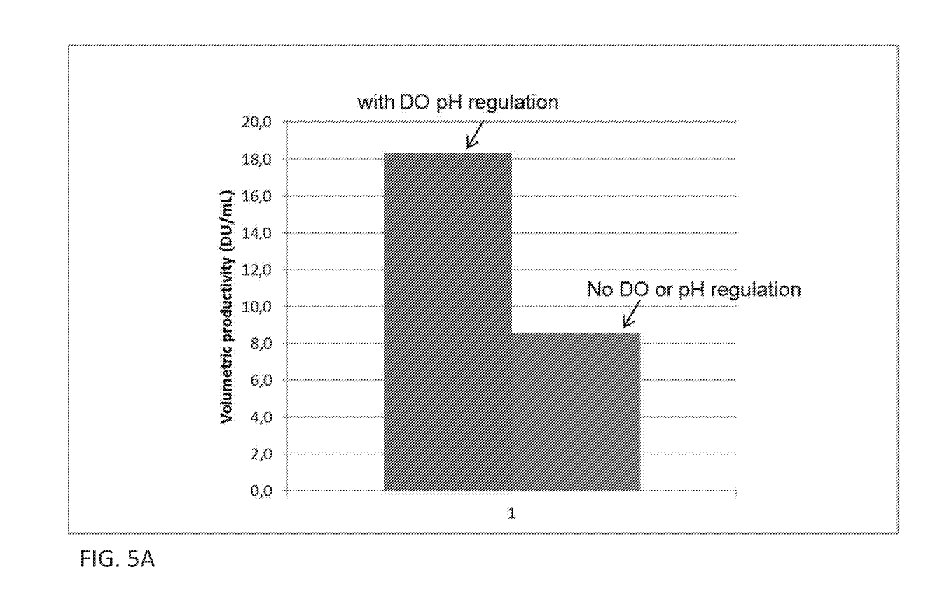

As shown in FIG. 4, production of D-antigen was stable under both sets of conditions. An ˜15% loss of D-antigen was observed for the iCELLis® NANO, but this loss did not decrease over time. These data suggest that the dynamic environment of the iCELLis® NANO is not responsible for lower productivity compared to T-flask use.

Next, the effect on pH and DO regulation on growth in the iCELLis® NANO system was assessed. Cultures were grown in iCELLis® NANO with and without active regulation of pH and DO. A mixture of air/CO₂ (5% v/v) was injected into the bioreactor at a flow of 30 mL/min for the condition without active regulation. The CDIs for the “No regulation” and regulated conditions were 0.18×10⁶ and 0.16×10⁶ cells/cm², respectively.

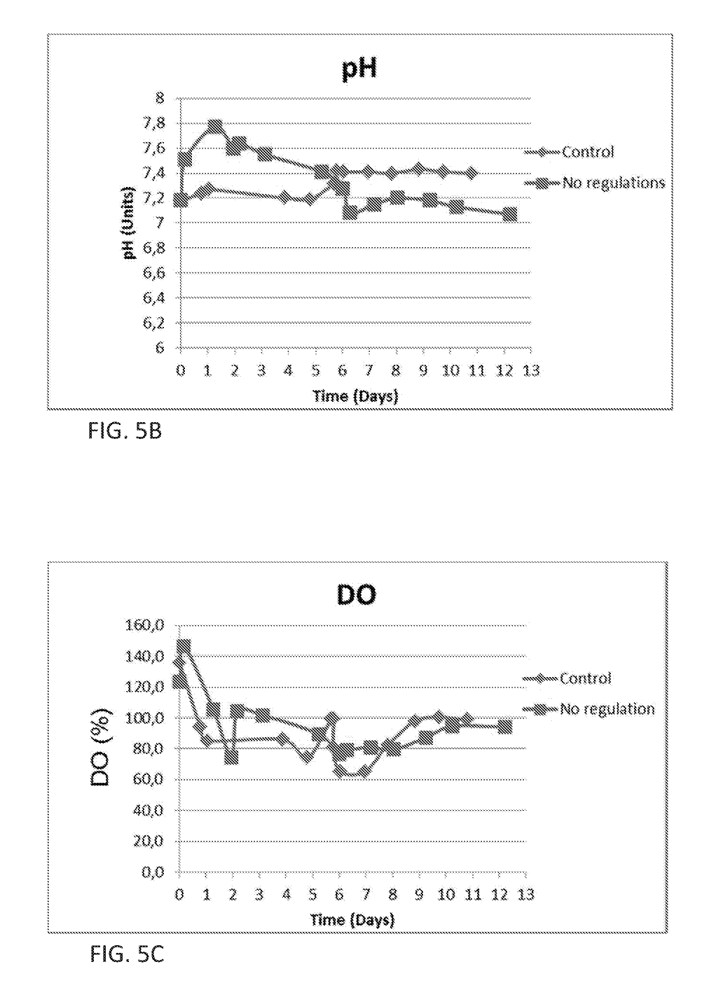

FIG. 5A shows that productivity was much higher with active pH/DO regulation than without. FIGS. 5B & 5C show the pH and DO levels of the control and “no regulation” conditions over time (infection took place around day 6 in both conditions). These results highlight the importance of active pH/DO regulation in the iCELLis® NANO system.

### Example 4: Effect of Cell Lysis on Pollovirus Production Using ICELLis NANO®

The effect of cell lysis on recovery was examined.

Results

Cells were grown and assayed each day after infection with poliovirus for volumetric productivity of extra- or intra-cellular D-antigen. Samples were taken from each culture, freeze-thawed to lyse the cells, and D-antigen content was measured. Extracellular D-antigen content was measured without performing the freeze-thaw step. Intracellular D-antigen was calculated as: (D-antigen level of sample after freeze-thawed process)−(D-antigen level of sample without freeze-thaw process).

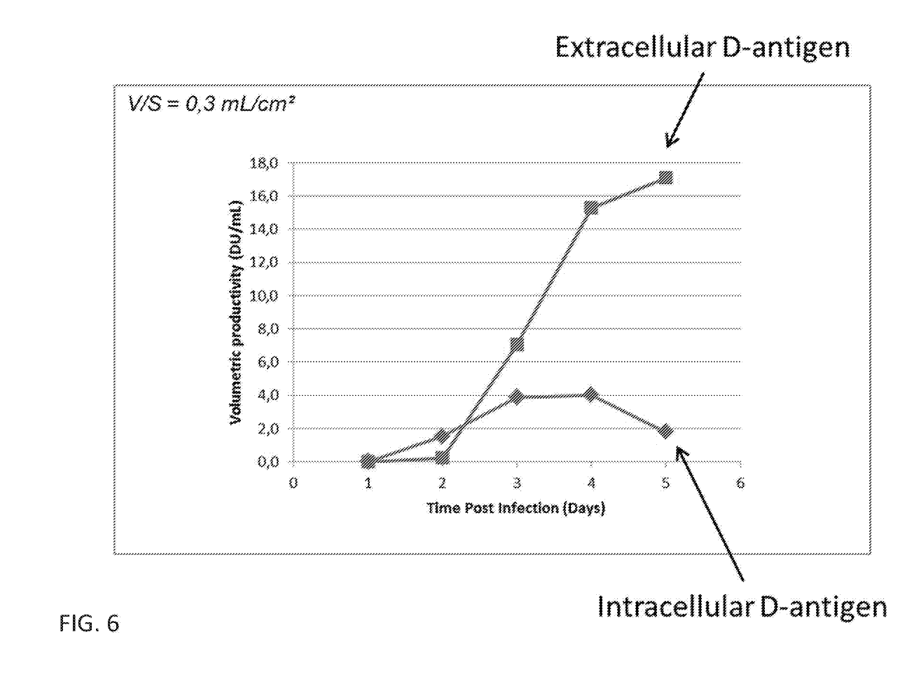

FIG. 6 shows the production of extracellular and intracellular D-antigen over time. For example, at 4 days post-infection, up to 20% of total D-antigen was still found intracellularly. These results indicate that cell lysis at harvest may improve virus recovery, e.g., 10-20% additional virus may be recovered by cell lysis.

### Example 5: Effect of Cell Metabolic State on Poliovirus Production Using ICELLis NANO®

The effect of metabolism (e.g., glucose consumption and/or lactate production) on cell productivity was examined.

Results

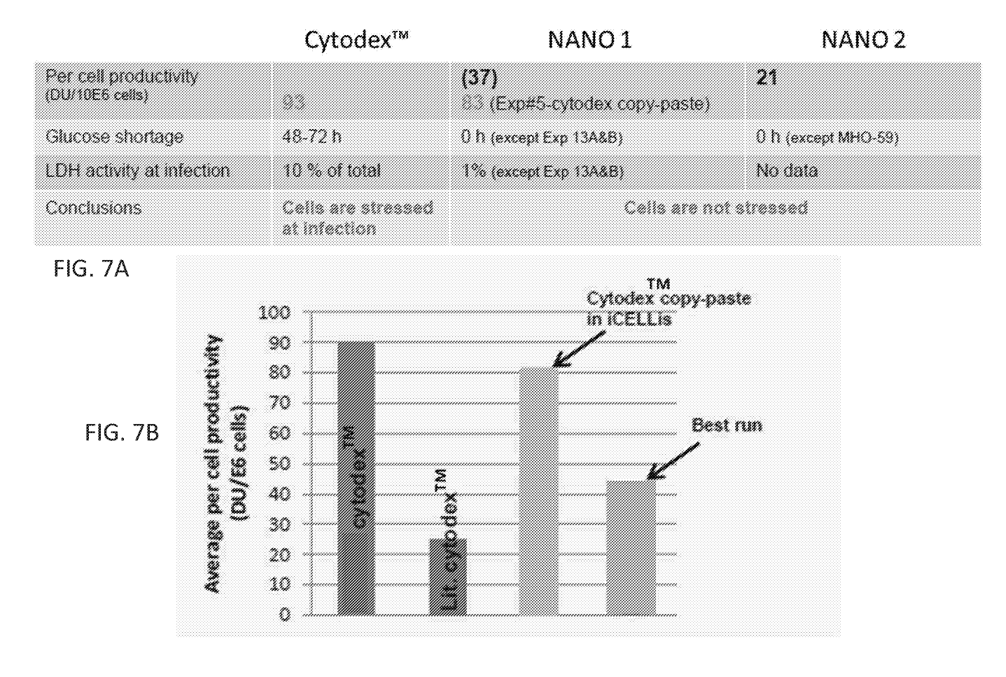

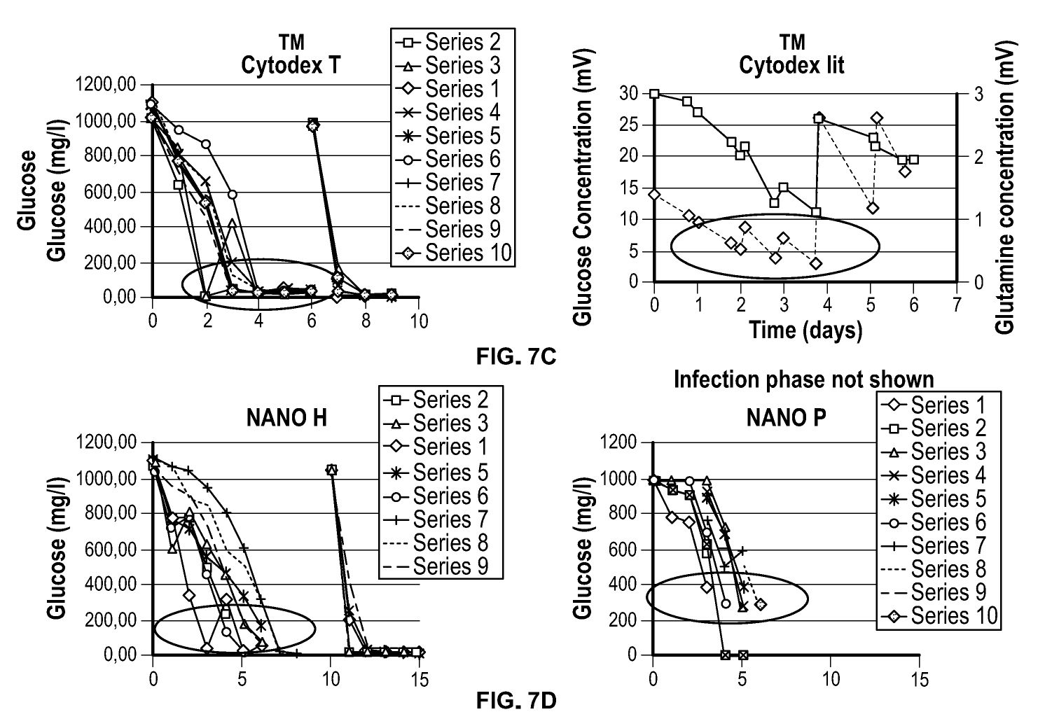

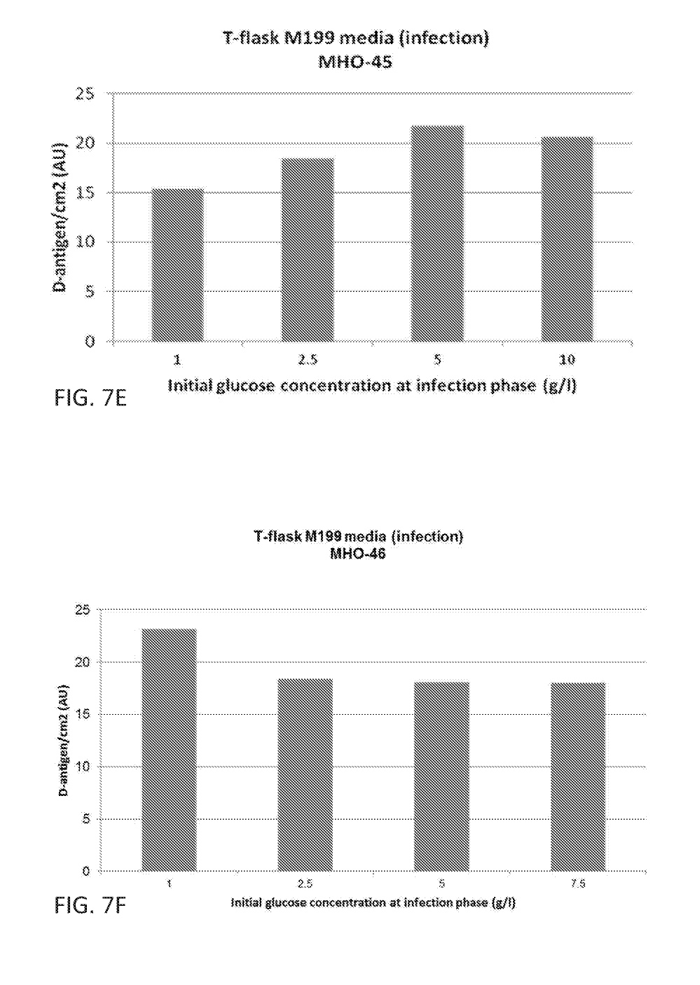

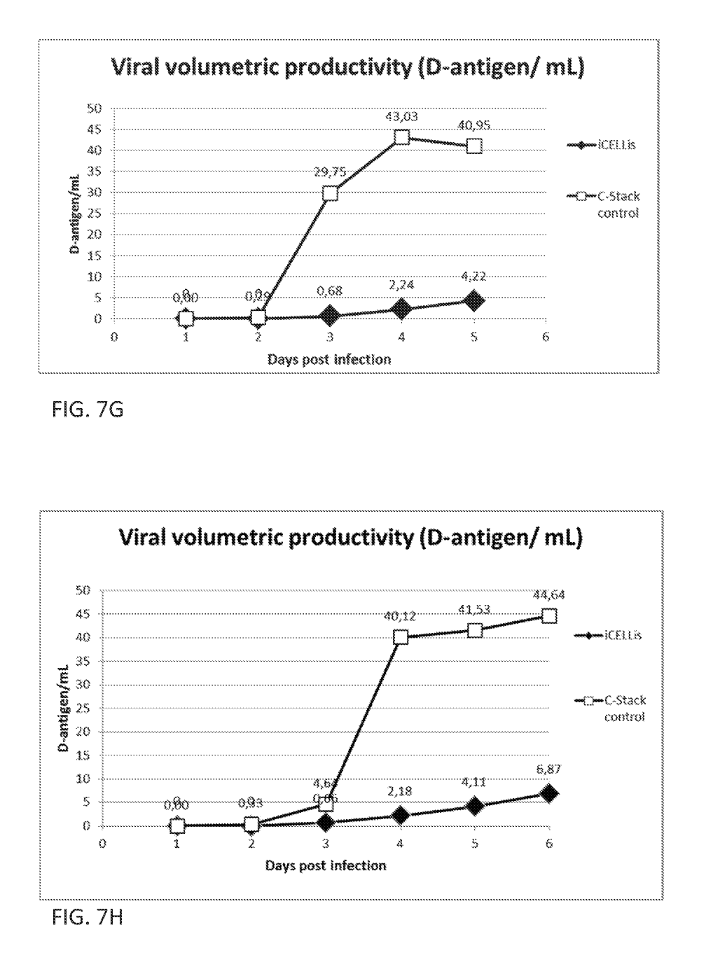

Cells were cultured and infected with virus as described above. Glucose and lactate were measured as described above. Per cell productivity (DU/10⁶ cells), glucose shortage, and LDH activity at infection were measured for cells grown on microcarriers (e.g., CYTODEX™ microcarriers from GE Healthcare Life Sciences) and compared to two batches of cells grown using the iCELLis NANO® system (FIG. 7A). If glucose levels fell below 250 mg/L during culturing, glucose shortage/depletion would be observed on the following day.

The results demonstrated increased per cell productivity of cells grown on microcarriers as compared to an unoptimized iCELLis NANO® system-based process. To determine whether the difference in productivity may be determined by metabolic differences between cells cultured in the two systems, glucose shortage and LDH activity at infection were examined. Compared to cells grown on microcarriers, which experienced glucose shortage 48-72 hours prior to infection, cells grown in the iCELLis NANO® system experienced no glucose shortage. Cells grown in the iCELLis NANO® system also showed a ten-fold reduction in LDH activity at infection compared to cells grown on microcarriers. These results demonstrate metabolic differences between the two culture systems. Without wishing to be bound to theory, these results suggest that metabolic stress (e.g., at infection) and/or higher cell density may lead to increased productivity.

Next, average per cell productivity was measured in cells grown on microcarriers according to the present disclosure and compared to cells grown on microcarriers according to protocols in the scientific literature. As shown in FIG. 7B, the cytodex-based culturing protocol out-performed culturing using a culture found in the scientific literature (cf. “cytodex” and “lit. cytodex” bars). Using the same temperature and pH conditions of the “cytodex” protocol in an iCELLis NANO® system resulted in a slight decrease in productivity (cf. “cytodex copy-paste in iCELLis” with “cytodex”). The best run using the unoptimized iCELLis NANO® conditions is shown as “best run.”

Glucose level in the culture medium was next compared between these protocols (FIG. 7C) and also compared against cell productivity in the iCELLis NANO® system (FIG. 7D). Compared to microcarrier culture, cell stress was greatly reduced in the iCELLis NANO® system. Without wishing to be bound to theory, these results suggest that cell stress and higher cell density may lead to increased productivity, and that considering cell metabolism, iCELLis is more preferable for cell culture because more glucose, critical for cell metabolism, is remaining in cell culture medium later on day 3 in iCELLis than cytodex.

Next, as shown in FIGS. 7E & 7F, the impact of glucose addition during infection on productivity was examined. Two cultures were grown in T-flasks at initial glucose concentrations (in the cell culture medium, M199) ranging from 1 to 10 g/L. Cells were grown in growth phase in T-flasks with a V/S of 0.5 mL/cm². The glucose concentration at infection phase is indicated. One culture showed a positive effect of higher glucose concentrations on productivity (FIG. 7E), whereas the other culture showed no impact (FIG. 7F). Since addition of glucose had no negative effect on productivity, and without wishing to be bound to theory, it is thought that maintaining glucose addition (e.g., at a level of 4.5 g/L) may be advantageous to avoid glucose shortage during infection phase.

FIG. 7G shows the effect of glucose shortage prior to infection on cell productivity. Cells were grown in growth phase in the iCELLis NANO® system with 24 hours of complete glucose shortage prior to infection. At infection phase (with cells at 0.3 mL/cm²), no extra glucose was added. Since anticipated productivity in the iCELLis NANO® system was ˜20 DU/mL, glucose shortage was observed to have a potentially negative effect on productivity (FIG. 7G). No negative impact was observed on cells grown in the control cell-stack culture system.

FIG. 7H shows the effect of adding extra glucose at infection on cell productivity. Cells were grown in growth phase in the iCELLis NANO® system with no glucose shortage prior to infection. At infection phase (with cells at 0.3 mL/cm²), extra glucose was added (4.5 g/L with 5% FBS). Since anticipated productivity in the iCELLis NANO® system was ˜20 DU/mL, the addition of extra glucose at infection was observed to have a potentially negative effect on productivity (FIG. 7H). No negative impact was observed on cells grown in the control cell-stack culture system. In summary, addition of extra glucose with FBS to virus culture medium did not improve poliovirus yield. Similarly, glucose deprivation in growth phase did not improve poliovirus yield.

### Example 6: Effect of Polysorbate on Viral Harvest

The effect of polysorbate addition on viral harvest yields was examined.

Results

Virus was grown in the iCELLis NANO® as described above with active pH/DO regulation and agitation. One hour prior to harvest, 0.05% TWEEN®-80 was added to the culture. After harvest, the iCELLis NANO® was rinsed with 10 mM Tris buffer (pH 7.4). D-antigen production was measured upon initial harvest before adding TWEEN®-80, after addition of TWEEN®-80, and after rinsing the NANO with Tris buffer.

FIG. 8A shows that addition of polysorbate (in this example, TWEEN®-80) prior to harvest resulted in a two-fold increase in the amount of recovered virus. Among the total harvest (measured by DU production), 45% was captured before addition of TWEEN®-80, 46% was captured after addition of TWEEN®-80, and 9% was recovered after rinsing with Tris buffer. These results demonstrate significant improvements in productivity as a result of addition of polysorbate before or during viral harvest.

A comparison between two processes (Process 1 and Process 2) was performed and is summarized in FIG. 8B. Differences in Process 2 as compared to Process 1 are bolded. These data suggest that using a high MOI or a lower V/S ratio at infection has little impact on productivity. For example, using a V/S ratio at infection of 0.1 mL/cm² or 0.3 mL/cm² had no effect on overall productivity.

Productivity of processes 1 and 2 are shown in FIG. 8C. In the “Tween during infection” condition, 0.05% TWEEN®-80 was present in the infection medium. In the “Repeat” condition, the same infection medium was used without TWEEN®-80, resulting in a much lower amount of virus recovered in the initial harvest. The “Extra recovery Tween” portion of the “Repeat” harvest indicates D-antigen titer recovered after washing the bioreactor with a rinsing buffer containing TWEEN®-80 after the initial harvest. These data demonstrate the increase in yield achieved by including surfactant in the infection medium.

### Example 7: Comparison of Upstream Process Improvements on Different Poliovirus Strains

Virus production was measured for three different strains—S1, S2, and S3—using the upstream process improvements described above. Strains used for each serotype were: Type I: LSc,2ab, Type II: P712,Ch,2ab, Type III: Leon,12_{a1b}.

Results

Three viral serotypes were produced in the iCELLis NANO® system using the methods described above. The results are shown below in Table A.

TABLE A
S1, S2, and S3 virus production using the iCELLis NANO ®

| | | | Exp-A | | Exp-A2 | |
|---|---|---|---|---|---|---|
| | | Target | Serotype | | | |
| | | — | S2 | S3 | S1 | S2 |
| Virus | Volume (L) | 2.40 | 2.40 | 2.40 | 2.40 | 2.40 |
| Culture | Vol/Surface Ratio | 0.30 | 0.30 | 0.30 | 0.30 | 0.30 |
| Medium | (mL/cm²) | | | | | |
| Cell density at Infection | | — | 1.50 | 1.90 | 2.01 | 2.34 |
| (*10⁵ cells/cm²) | | | | | | |
| D antigen | Titer (DU/mL) | 88 | 8 | 126 | 44 | 25* |
| | Batch Total (DU) | 211,200 | 19,080 | 302,208 | 104,890 | 61,001 |
| | /Surface Ratio | 26.40 | 2.39 | 37.77 | 13.11 | 7.63 |
| | (DU/cm²) | | | | | |
| | /Goal Ratio | 1.00 | 0.09 | 1.43 | 0.50 | 0.29 |
| | /Cell Count | — | 1.59 | 19.88 | 6.52 | 3.26 |
| | Ratio (*10⁻⁵ | | | | | |
| | DU/cells) | | | | | |

*Titer was 33 DU/mL after 4-hour incubation with 0.05% TWEEN ®-80.

The only parameter differences between Experiment A (Exp-A) and Experiment B (Exp-B) were the cell density at infection (shown above) and the circulation of virus culture medium on day 1 and day 2 of infection. Circulation was started on day 1 for Exp-A S2 and on day 2 for Exp-A S3 and Exp-A2 S1 and S2.

These results indicate that the upstream process improvements described above were effective in improving the yield of three different poliovirus serotypes. Overall, each serotype was harvested in 2,400 mL volume of FBS-containing culture medium. Total D-antigen production was 104,890 DU for S1, 61,001 DU for S2, and 302,208 DU for S3. D-antigen concentration (DU/mL at harvest) was 43.7 for S1, 33.3 for S2, and 125.9 for S3.

### Example 8: Improvement of Downstream Processing Steps for Pollovirus Production Using iCELLis NANO®

Existing downstream processing protocols are time- and resource-intensive. For example, FIG. 9A compares the improved protocol described herein with an existing protocol from U.S. Pat. No. 8,753,646. Rather than requiring multiple filtration and ultracentrifugation steps (as with the existing protocol), the improved process described herein uses anion exchange chromatography (e.g., using the Mustang-Q membrane from Pall Corporation, Pt. Washington, N.Y.) and cation exchange chromatography (e.g., using the Mustang-S membrane from Pall Corporation, Pt. Washington, N.Y.). This improved process allows for a more streamlined and cost-efficient downstream processing scheme.

A flow diagram illustrating the improved downstream process for virus production inactivation is shown in FIG. 9B. The following Examples detail the validation and optimization of this improved purification process, as well as the combination of this process with virus production using the iCELLis NANO® system.

Results

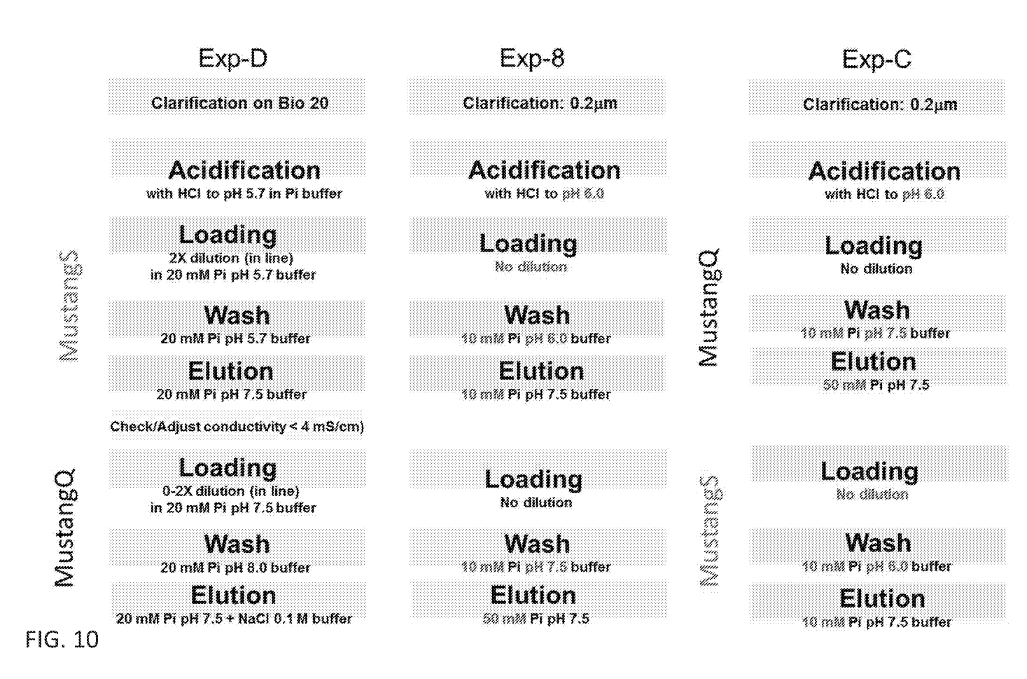

Three experimental designs were employed to test the effect of various downstream processing parameters on virus production (FIG. 10). The results of these experiments are shown in Tables B and C below.

TABLE B
Results from experiments C, D, and 8.

| | | | Exp-8 | Exp-C | Exp-D | |
|---|---|---|---|---|---|---|
| | | Target | Serotype | | | |
| Parameter | | — | S2 | S2 | S2 | S3 |
| 1^{st}: Mustang-S | Bed volume (mL) | — | 0.86 | 0.86 | 0.86 | 0.86 |
| 2^{nd}: Mustang-Q | Loaded total D-antigen | — | 18,716 | 7,228 | 8,901 | 26,888 |
| | (DU) | | | | | |
| | Recovered D-antigen | — | 11,923 | < Lower | 7,284 | 146 |
| | (DU) | | | Limit | | |
| | D-antigen recovery rate | >50.0 | 63.7 | N/A | 81.8 | 0.5 |
| | (%) | | | | | |
| | Total Protein Content | — | 126 | — | 899 | 495 |
| | (mg) | | | | | |
| | Total Protein/D-antigen | | 0.017 | N/A | 0.123 | 3.379 |
| | Ratio (mg/DU) | | | | | |

TABLE C
Results from experiments C, D, and 8.

| | | | Exp-8 | Exp-C | Exp-D | |
|---|---|---|---|---|---|---|
| | | Target | Serotype | | | |
| Parameter | | — | S2 | S2 | S2 | S3 |
| Mustang-S | Bed Volume of | — | 0.86 | 0.86 | 0.86 | 0.86 |
| | Acrodiscs (mL) | | | | | |
| | Loaded total D- | — | 18,716 | 7,228 | 8,901 | 26,888 |
| | antigen (DU) | | | | | |
| | Recovered D-antigen | — | 13,683 | 27 | 8,244 | 4,40-4 |
| | (DU) | | | | | |
| | D-antigen recovery | — | 73.1 | 0.4 | 92.6 | 16.4 |
| | rate (%) | | | | | |
| | Total Protein Content | — | 1,191 | — | 3,968 | 2,844 |
| | (mg) | | | | | |
| | Total Protein/D- | — | 0.087 | — | 0.481 | 0.646 |
| | antigen Ratio | | | | | |
| | (mg/DU) | | | | | |
| Mustang-Q | Bed Volume of | | 0.86 | 0.86 | 0.86 | 0.86 |
| | Acrodiscs (mL) | | | | | |
| | Recovered D-antigen | — | 11,923 | < Lower | 7,284 | 146 |
| | (DU) | | | Limit | | |
| | D-antigen recovery | — | 90.1 | N/A | 81.8 | 3.3 |
| | rate (%) | | | | | |
| | Total Protein Content | — | 126 | — | 899 | 495 |
| | (mg) | | | | | |
| | Total Protein/D- | — | 0.019 | N/A | 0.123 | 3.379 |
| | antigen Ratio | | | | | |
| | (mg/DU) | | | | | |

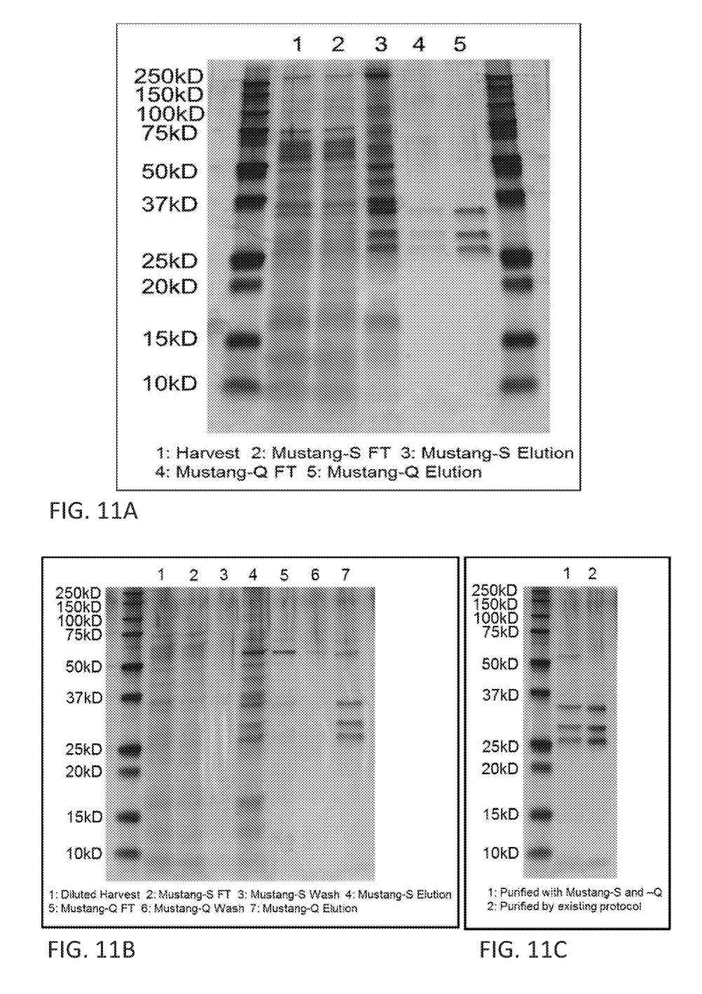

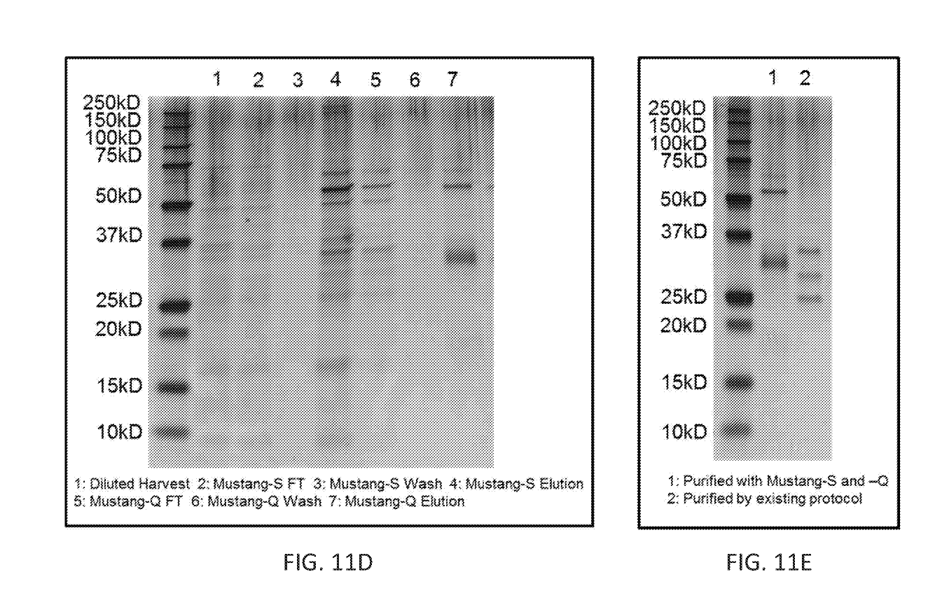

Products of various purification steps from these experiments are shown in FIGS. 11A-11E. The purification of S2 virus from Experiment 8 (FIG. 11A) is similar to that shown in FIG. 3B of Thomassen, Y. E. et al. (2013) PLoS ONE 8:e83374. This indicates that the eluent from the anion exchange membrane (lane 5 in FIG. 11A) contains poliovirus, and that this poliovirus suspension was sufficiently purified using sequential cation and anion exchange chromatography steps. These results demonstrate that poliovirus was sufficiently purified using cation/anion exchange chromatography with suitable parameter settings as described herein. FIG. 11B shows that although certain parameters were changed in the purification process to improve D-antigen recovery, extra protein (e.g., at 60 kD) was not completely removed. As shown in FIG. 11D, the S3 strain requires a different pH for cation exchange loading than strains S1 and S2. FIG. 11E shows that at the same pH, little S3 bound to the cation exchange membrane. The band shown in lane 7 around 30 kD is not an S3 poliovirus component.

These results demonstrate that purification of S2 virus from Experiment 8 (FIG. 11C) was similar to that produced using the purification method of the existing protocol (cf. lanes 1 and 2 from FIG. 11C). The existing protocol (e.g., as described in U.S. Pat. No. 8,753,646) is summarized in FIG. 9B (“Existing”). Therefore, the streamlined purification scheme outlined in FIGS. 9B & 10 yields high purity with bands similar to those purified using prior technology, such as the existing protocol, chromatography, or the protocol described in Thomassen, Y. E. et al. (2013) PLoS ONE 8:e83374. However, purification of S3 virus requires a different pH for cation exchange loading in order to produce purified virus similar to the existing protocol (cf. lanes 1 and 2 from FIG. 11E). Improvements to downstream processing to facilitate purification of S3 virus are further described herein.

A summary of the purification process results for viral serotypes S1, S2, and S3 is provided in Table D below.

TABLE D
Summary of purification of three viral serotypes

| Parameter | | S1 | S2 | S3 |
|---|---|---|---|---|
| Mustang-S | Bed Volume of Acrodiscs (mL) | 0.86 | 0.86 | 0.86 |
| | Loaded total D-antigen (DU) | 7,328 | 7,616 | 14,490 |
| | Recovered D-antigen (DU) | 6,363 | 5,421 | 12,376 |
| | D-antigen recovery rate (%) | 86.8 | 71.2 | 85.4 |
| | Total Protein Content (mg) | 1,752 | 2,199 | 2,361 |
| | Total Protein/D-antigen Ratio | 0.275 | 0.406 | 0.191 |
| | (mg/DU) | | | |
| Mustang-Q | Bed Volume of Acrodiscs (mL) | 0.86 | 0.86 | 0.86 |
| | Loaded total D-antigen (DU) | 5,727 | 4,979 | 11,138 |
| | Recovered D-antigen (DU) | 4,494 | 4,967 | 10,162 |
| | D-antigen recovery rate (%) | 78.5 | 99.8 | 91.2 |
| | Total Protein Content (mg) | 421 | 178 | 227 |
| | Total Protein/D-antigen Ratio | 0.094 | 0.036 | 0.022 |
| | (mg/DU) | | | |
| Overall | D-antigen recovery rate (%) | 61.3 | 65.2 | 70.1 |
| process | Total Protein/D-antigen Ratio | 0.094 | 0.036 | 0.022 |
| | (mg/DU) | | | |

These results demonstrate high recovery rates for anion and cation exchange steps, as well as the overall purification process, for all three viral serotypes. The total protein/D-antigen ratio in the purified drug substance was below 0.1. Thus, the final purified product of all serotypes should meet this specification after inactivation.

### Example 9: Identification of Capture Conditions for Anion and Cation Exchange Chromatography

Capture conditions such as pH, buffer composition, presence/absence of polysorbate, and dilution factor for improving D-antigen yield were tested.

Results

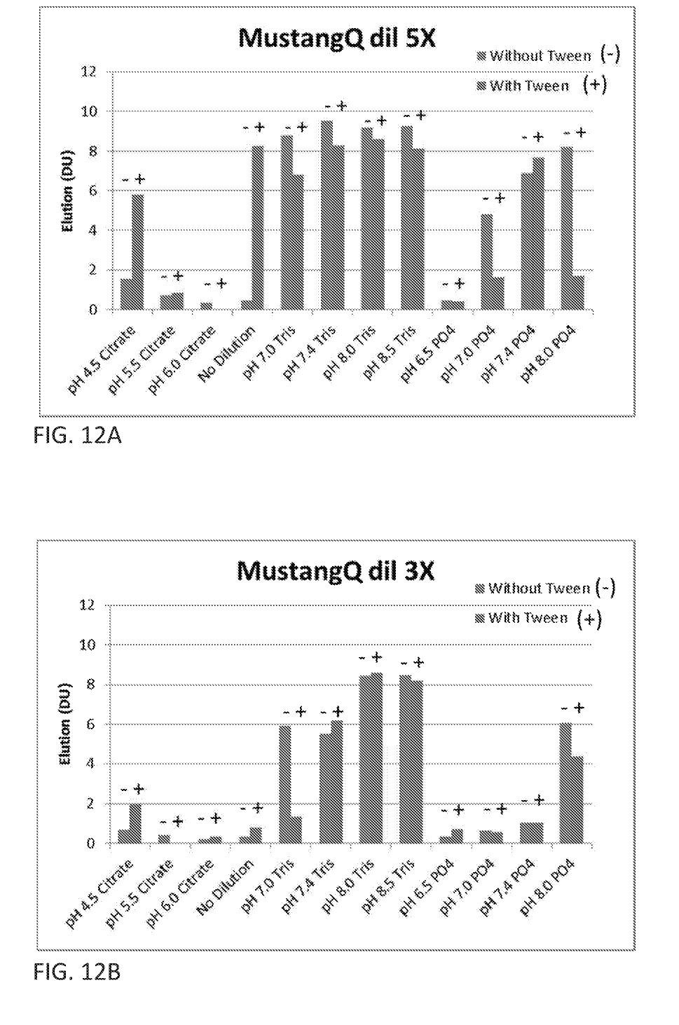

Various parameters of anion exchange chromatography (e.g., using a Mustang Q system, Pall Corporation) were assessed for effects on viral capture by screening conditions using a 96-well AcroPrep™ plate (Pall Corporation). Samples were ˜12 D-antigen units (theoretical) of clarified harvest. Samples were diluted 5× (FIG. 12A) or 3× (FIG. 12B), bound using the indicated conditions, and eluted in 0.75 mL with 0.75M NaCl in the indicated buffers. Elution fractions were analyzed for D-antigen (DU).

FIG. 12A shows the results of using a 5× dilution factor. Efficient capture (yield of ˜80% or more) was observed using Tris buffer at pH from 7.0-8.5. Compared to Tris buffer at comparable pH, capture was less efficient for most conditions using phosphate buffer. Some capture was seen at low pH, but with low efficiency. Polysorbate (0.05% TWEEN®-80) addition had a minor impact on capture efficiency under most conditions, but its addition may impact purity. Without wishing to be bound to theory, it is thought that the no dilution, with tween result is a discrepancy, most likely indicating no capture with no dilution.

FIG. 12B shows the results of using a 3× dilution factor. Compared to 5× dilution, reduction of the amount of dilution decreased capture efficiency under all conditions, notably except for Tris at pH 8.0 and 8.5. These results indicate room for dilution. In summary, these results indicate most efficient capture was observed using Tris buffer at pH 8.0-8.5, and that the dilution factor may be lowered to 3× or more. Without wishing to be bound to theory, it is thought that the no dilution, with tween result is a discrepancy, most likely indicating no capture with no dilution.

Next, various parameters of cation exchange chromatography (e.g., using a Mustang S system, Pall Corporation) were assessed for effects on viral capture by screening conditions as described above.

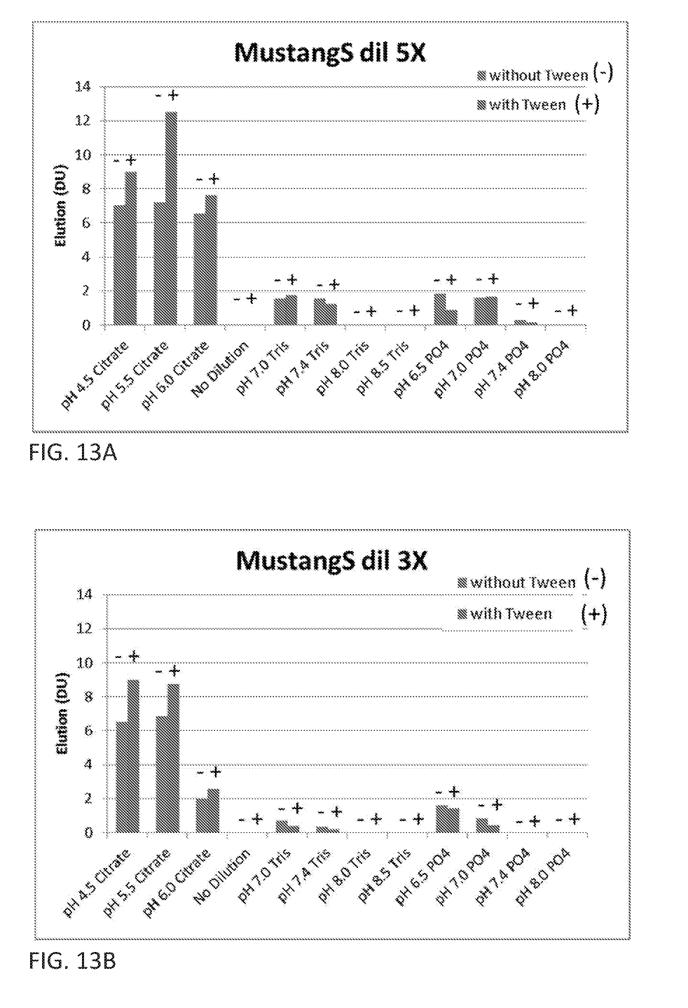

FIG. 13A shows the results of using a 5× dilution factor. The highest capture efficiency observed was ˜90% yield using pH 5.5 Citrate buffer and polysorbate. Citrate buffer at pH 4.5 or 6.0 caused less efficient capture. Use of polysorbate caused a slight increase in capture efficiency.

FIG. 13B shows the results of using a 3× dilution factor. Compared to 5× dilution, reduction of dilution factor to 3× decreased capture efficiency of all conditions except for Citrate buffer at pH 4.5 or 5.5. These results indicate room for dilution. In summary, these results indicate most efficient capture was observed using Citrate buffer at pH 4.5-5.5 with polysorbate, and that the dilution factor may be lowered to 3× or more.

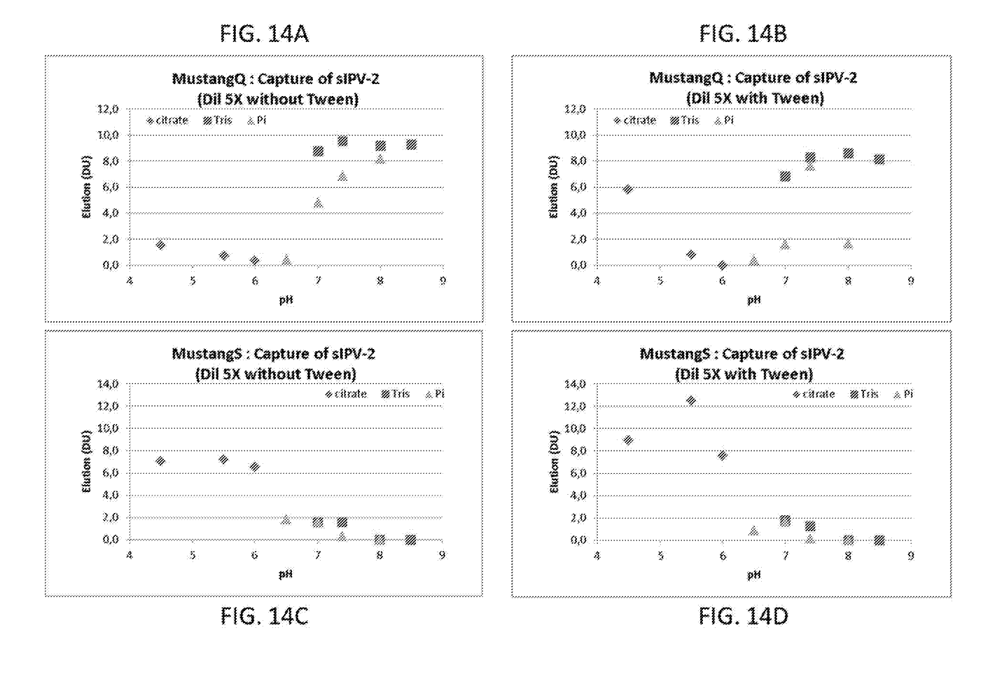

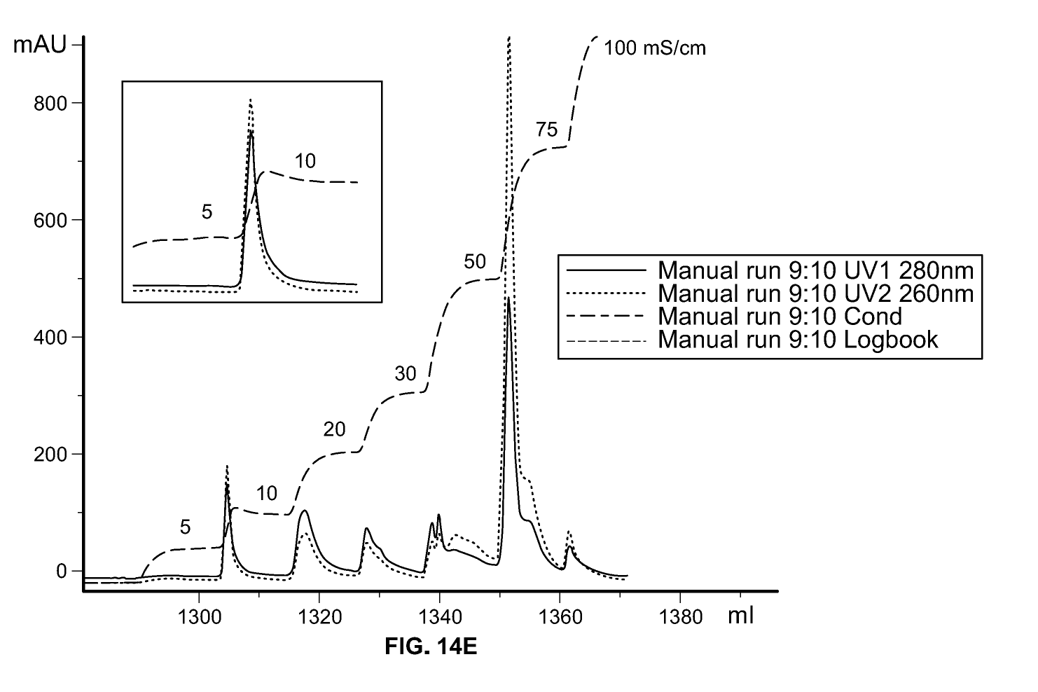

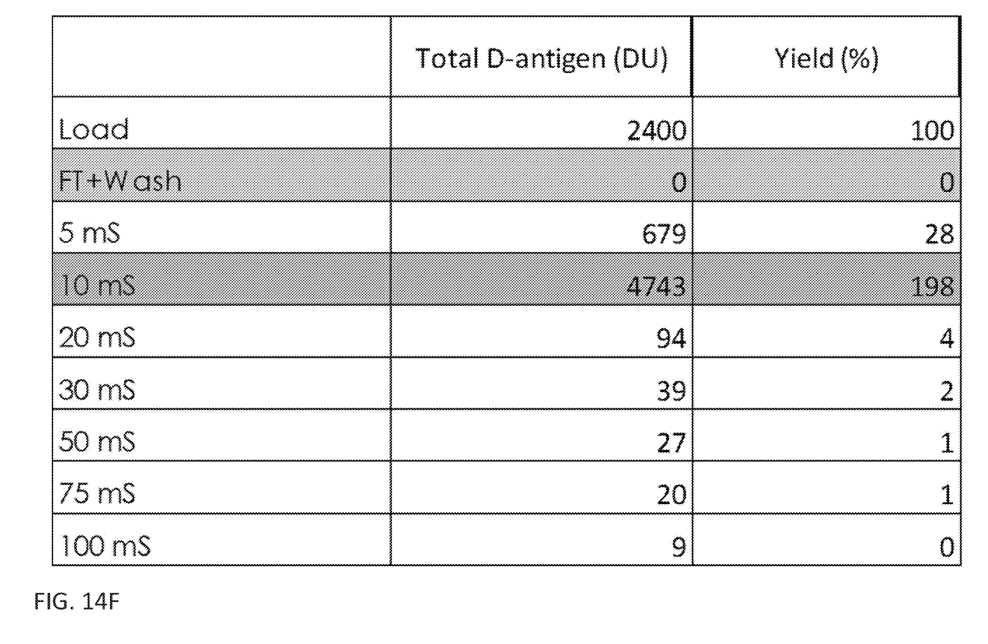

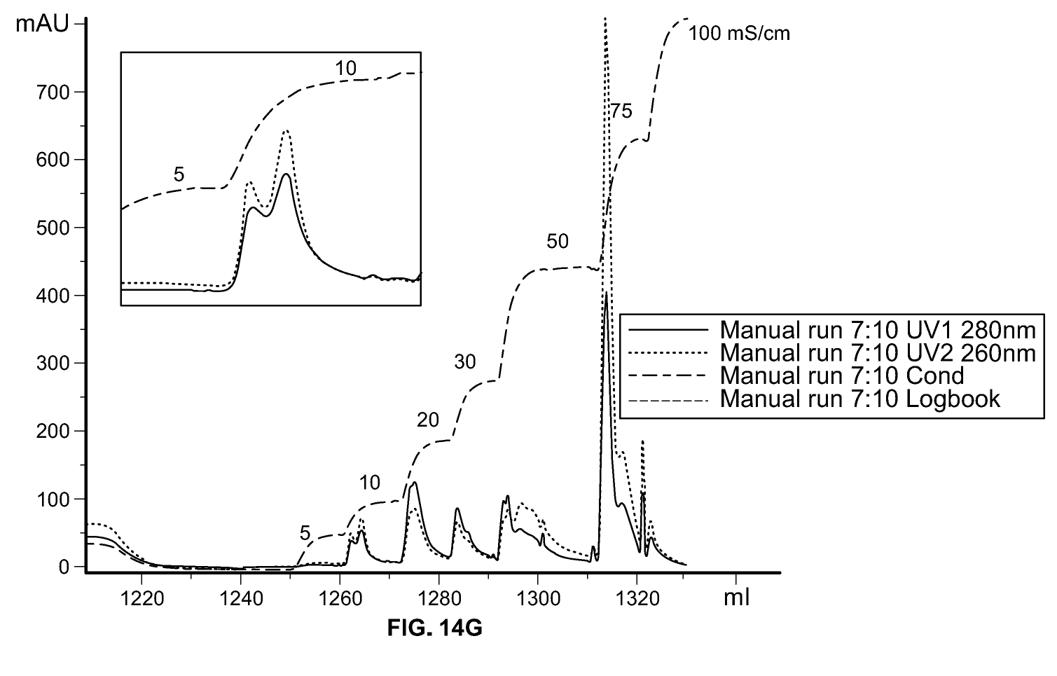

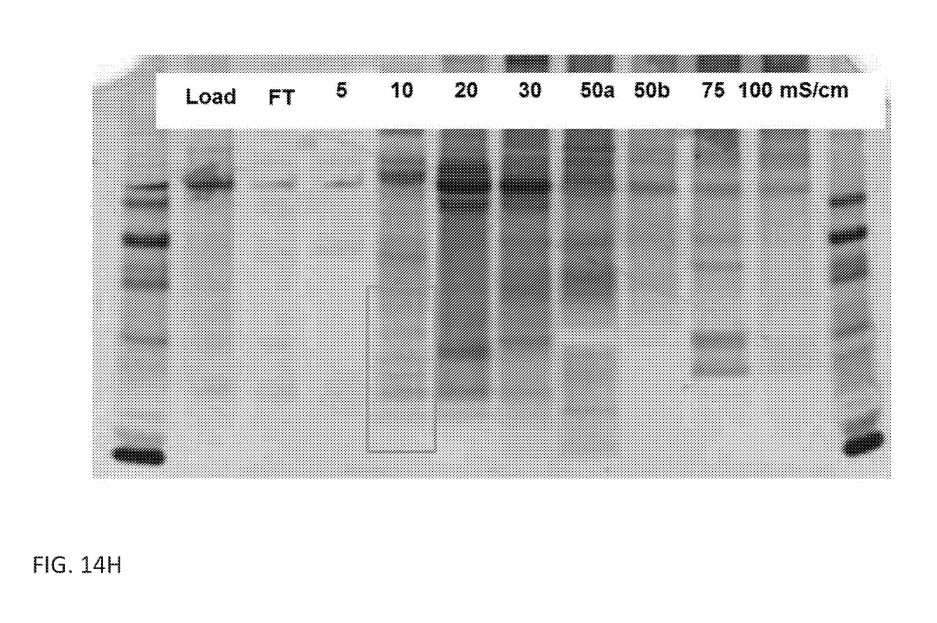

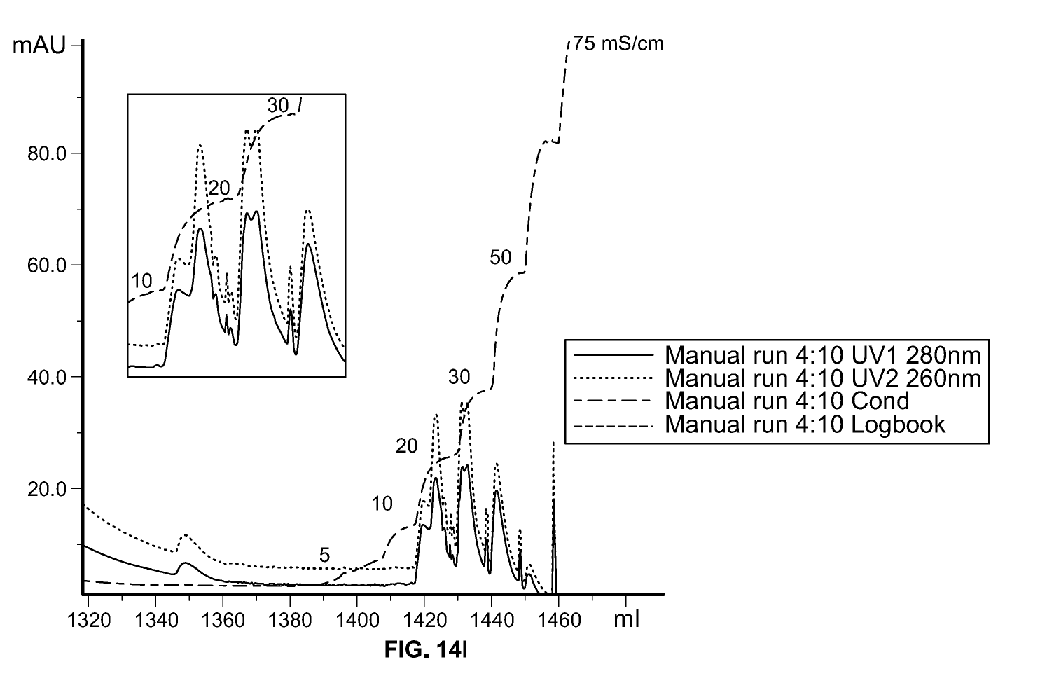

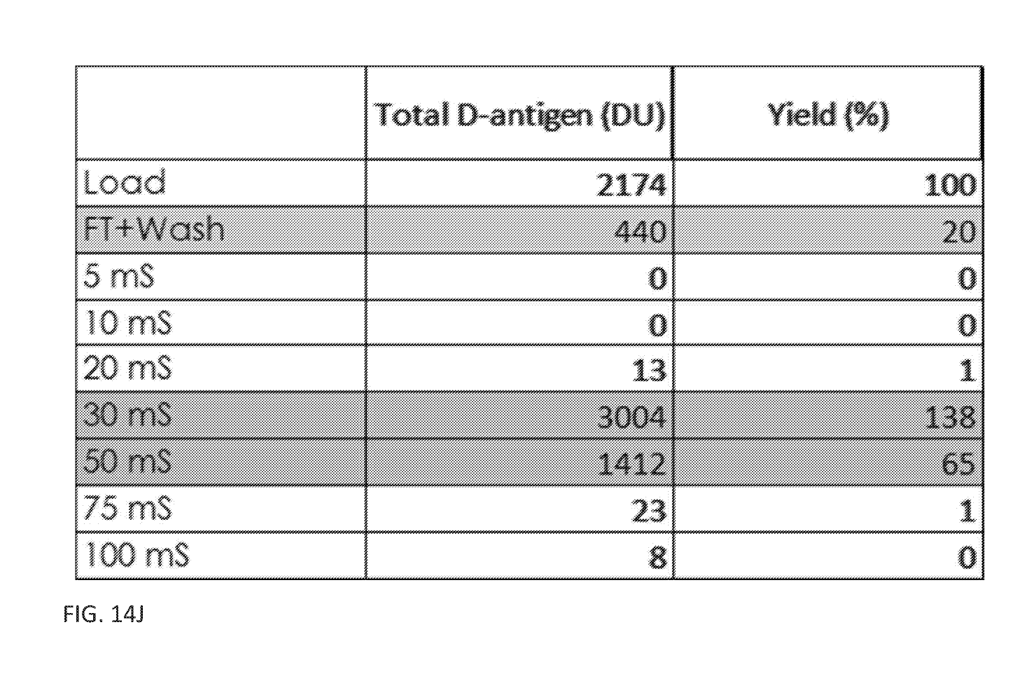

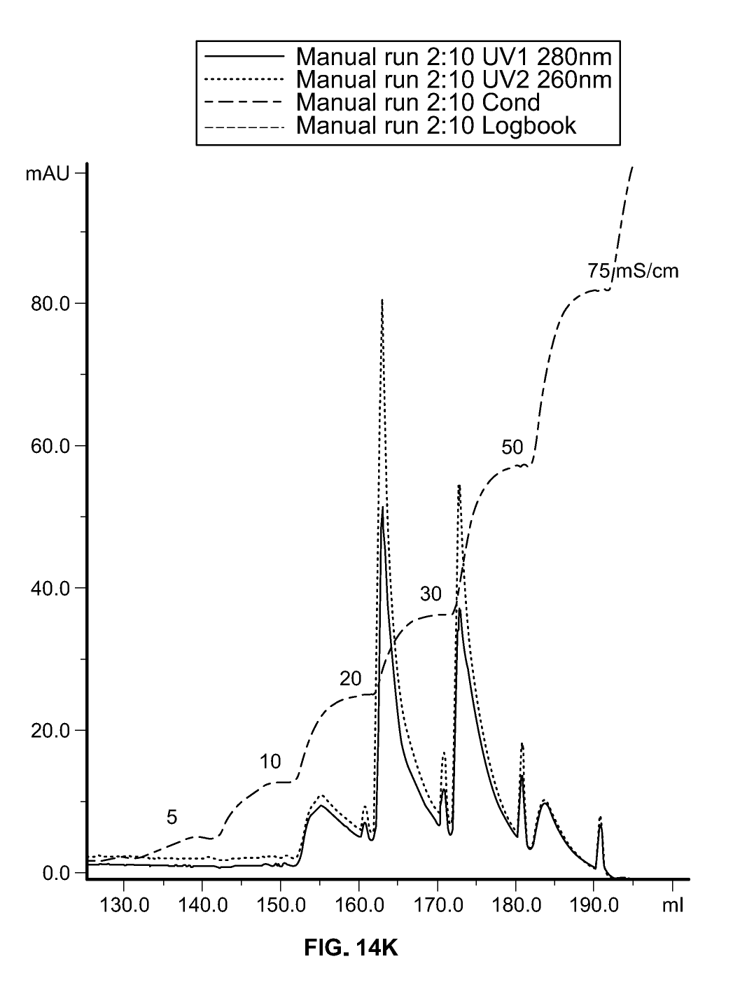

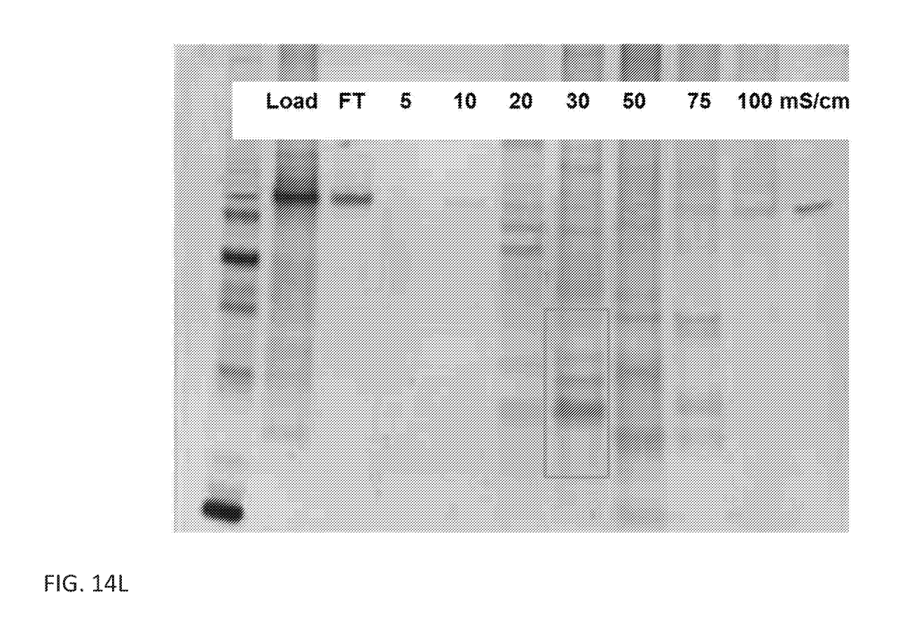

Binding efficiency as a function of pH (with and without polysorbate) using citrate, Tris, or phosphate buffer with cation and anion exchange chromatography are shown in FIGS. 14A-14D.

Anion exchange conditions were next tested for scaling up process size to the Mustang Q AcroDisc® scale. FIG. 14E shows the elution profile obtained with a 4× dilution factor using Tris pH 8.0 buffer without polysorbate. The total amount of virus (e.g., total D-antigen) obtained from each fraction, as well as the percentage yield, is shown in FIG. 14F. As shown in FIGS. 14E & 14F, no significant D-antigen was detected in the flow-through, and the yield was approximately 100%. Low conductivity (e.g., 10 mS) provided best elution.

FIG. 14G shows the elution profile obtained with a 4× dilution factor using Tris pH 8.0 buffer with polysorbate. Purity, as determined by SDS-PAGE silver staining, is shown in FIG. 14H. As shown in FIGS. 14G & 14H, no significant D-antigen was detected in the flow-through, and the purity was below 50%. Low conductivity (e.g., 10 mS) provided best elution.

Cation exchange conditions were next tested for scaling up process size to the Mustang S AcroDisc® scale. FIG. 14I shows the elution profile obtained with a 4× dilution factor using citrate buffer (pH 5.5) without polysorbate. The total amount of virus (e.g., total D-antigen) obtained from each fraction, as well as the percentage yield, is shown in FIG. 14J. As shown in FIGS. 14I & 14J, less than 10% of D-antigen was detected in the flow-through, and the yield was approximately 90%. Two peaks were observed at high elution conductivity.

FIG. 14K shows the elution profile obtained with a 4× dilution factor using citrate buffer (pH 5.5) with polysorbate. Purity, as determined by SDS-PAGE silver staining, is shown in FIG. 14L. As shown in FIGS. 14K & 14L, less than 5% of D-antigen was detected in the flow-through, and the yield was approximately 95%. Two peaks were observed at high elution conductivity. Purity was above 50%, as estimated by silver staining analysis of the peaks as resolved by SDS-PAGE.

A summary of scaling up the anion and cation exchange chromatography steps as described above is provided in Table D2.

TABLE D2
Summary of AcroDisc ® scale up results.

| Factor | MustangQ | MustangS |
|---|---|---|
| Yield | 100% | 90-95% |
| Est. max. loading | 5 mS/cm | 10-20 mS/cm |
| conductivity | 4X | 1-4X |
| Dilution factor | | |
| Purity | <50% | >50% |
| PROs | High yield (100%) | Lower dil. Factor (under |
| | | optimization) |
| | | Better purity |
| | | Better binding capacity |
| CONs | High dilution factor | 10% losses in FT |
| | Low purity | Additional acidification |
| | Lower binding capacity | step |

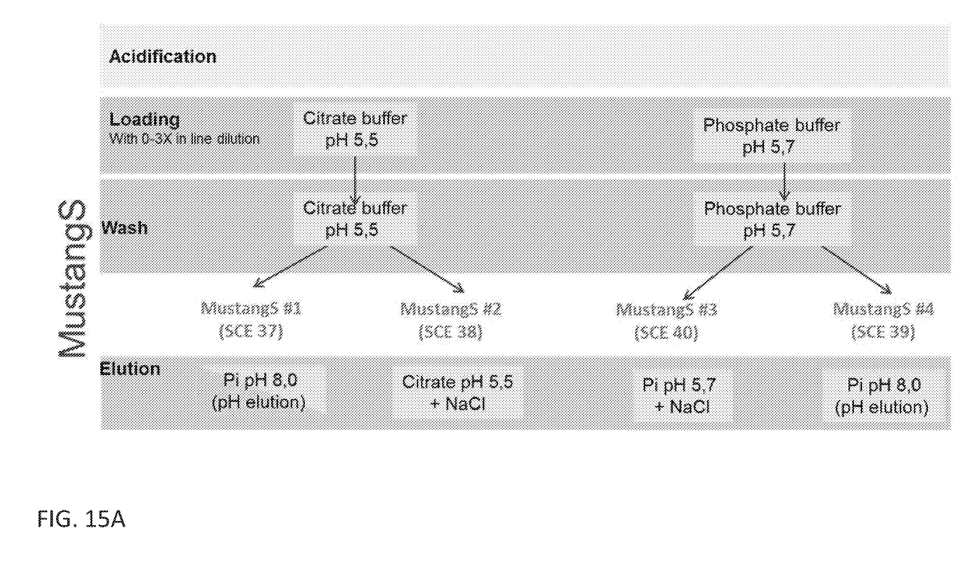

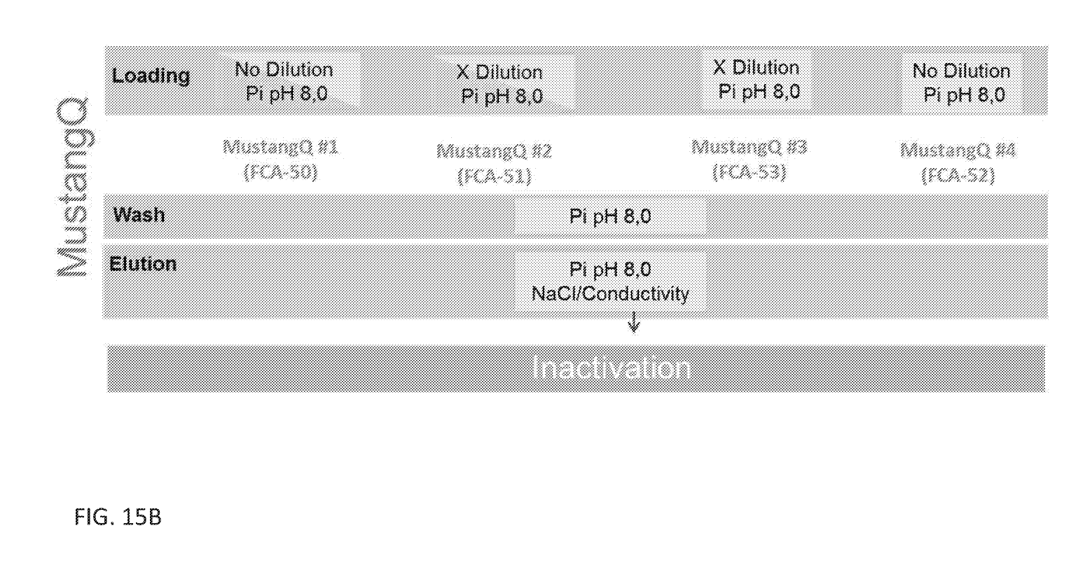

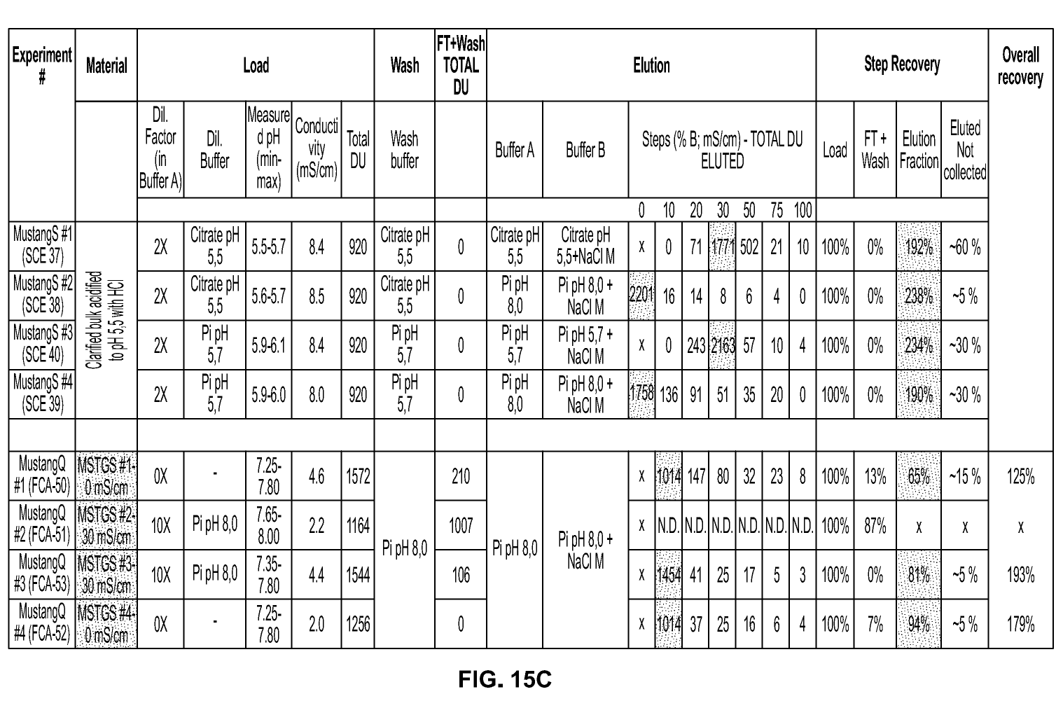

An experimental plan for testing various downstream processing parameters is shown in FIGS. 15A & 15B. Four combinations of cation exchange (FIG. 15A) and anion exchange (FIG. 15B) conditions were tested. The results of these tests are shown in FIG. 15C. These results indicate that for cation exchange chromatography, citrate or phosphate buffer can be used with little to no impact on yield. However, anion exchange yield appeared higher when phosphate buffer was used for the upstream cation exchange step, as compared to using citrate for the upstream cation exchange step (see step and overall recovery for MustangQ numbers 3 and 4 as compared to numbers 1 and 2).

Next, the effect of increasing buffer concentration was examined in combination with lowering cation exchange elution pH and anion exchange loading pH. These experiments were aimed at improving buffer capacity and targeting a more neutral pH.

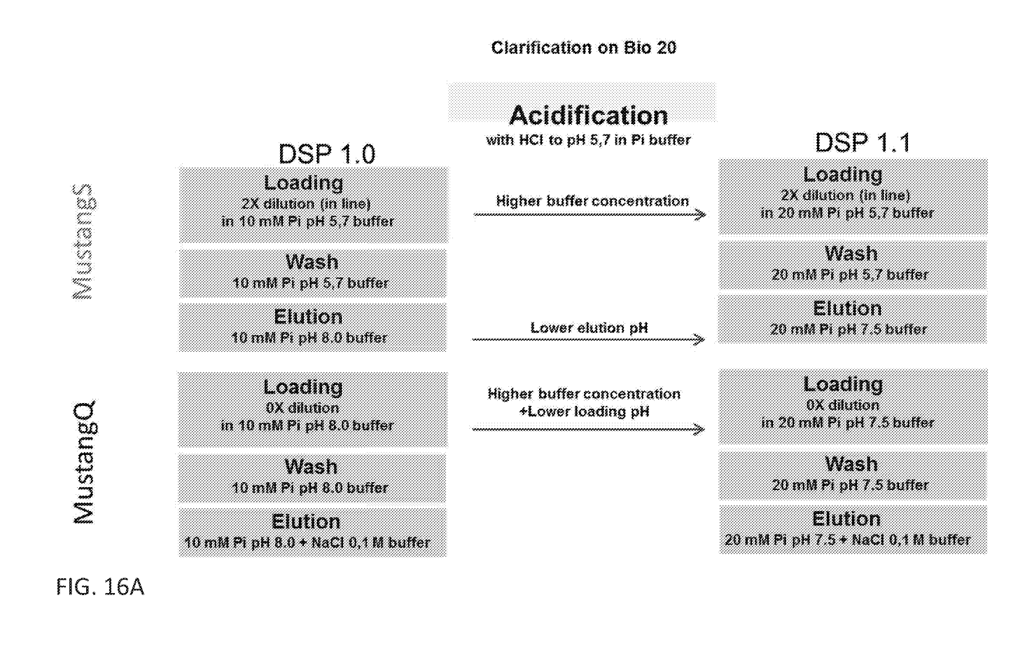

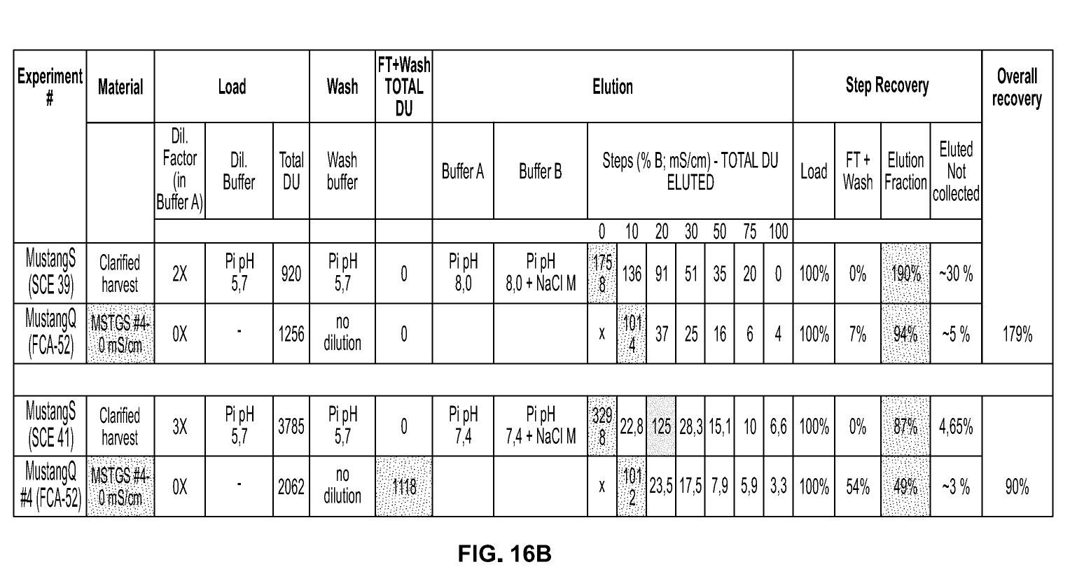

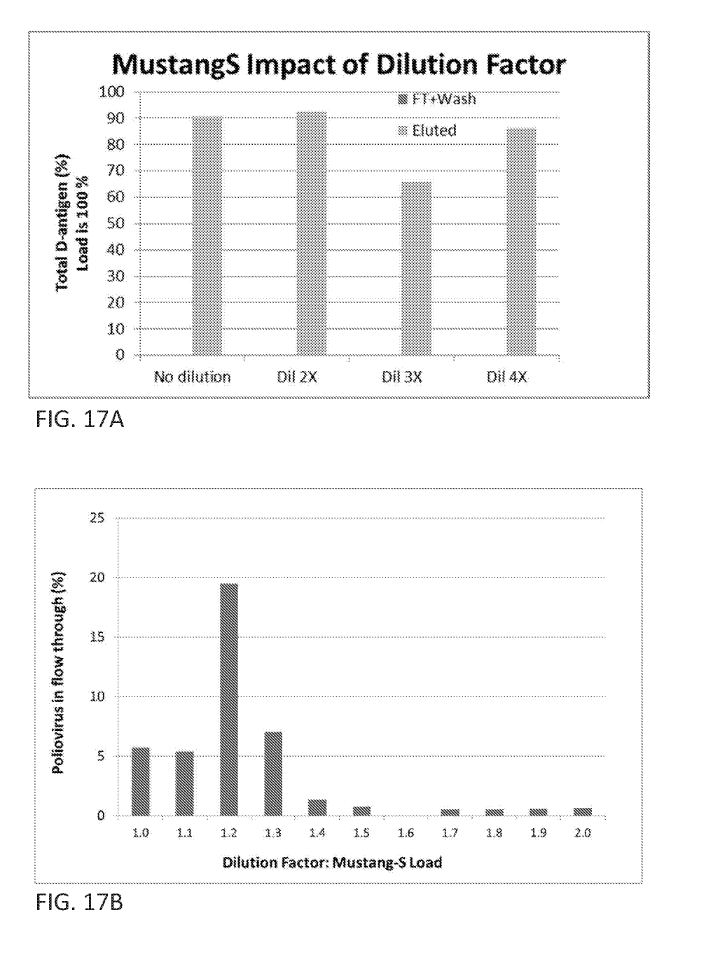

The experimental setup is shown in FIG. 16A. Performance with respect to recovery of DSP1.0, using the conditions described above, was compared with performance of DSP1.1, which used a higher buffer concentration for cation exchange column loading (20 mM vs. 10 mM phosphate buffer), a lower cation exchange elution pH (pH 7.5 vs. 8.0), and an anion exchange step with the same higher buffer concentration and same pH for loading. As shown in FIG. 16B, DSP1.1 led to a lower step and overall recovery. For cation exchange, a minor elution of sIPV at 20 mS was observed, which may be attributable to the lower elution pH. For anion exchange, 50% of sIPV in the flow-through was observed, which may be attributable to lower loading pH and higher buffer conductivity. These results suggest that a higher buffer pH and/or decreased buffer conductivity may lead to higher recovery.

Next, the dilution factor for the cation exchange chromatography step was tested for its effects on recovery. Mustang S AcroDisc® was used as the membrane. The in-line load was a clarified viral harvest (e.g., after clarification and acidification) with pH adjusted to 5.5. The dilution buffer was 10 mM Citrate. As shown in FIG. 17A, near complete recovery was achieved, with no recovery (as measured by DU) in the flow-through even under no dilution. Therefore, at pH 5.5, even OX dilution factor was acceptable, although at pH 5.7, a 2× dilution factor was acceptable. FIG. 17B shows that dilution factors below 1.3 resulted in the loss of more than 5% of S2 D-antigen in the flow-through.

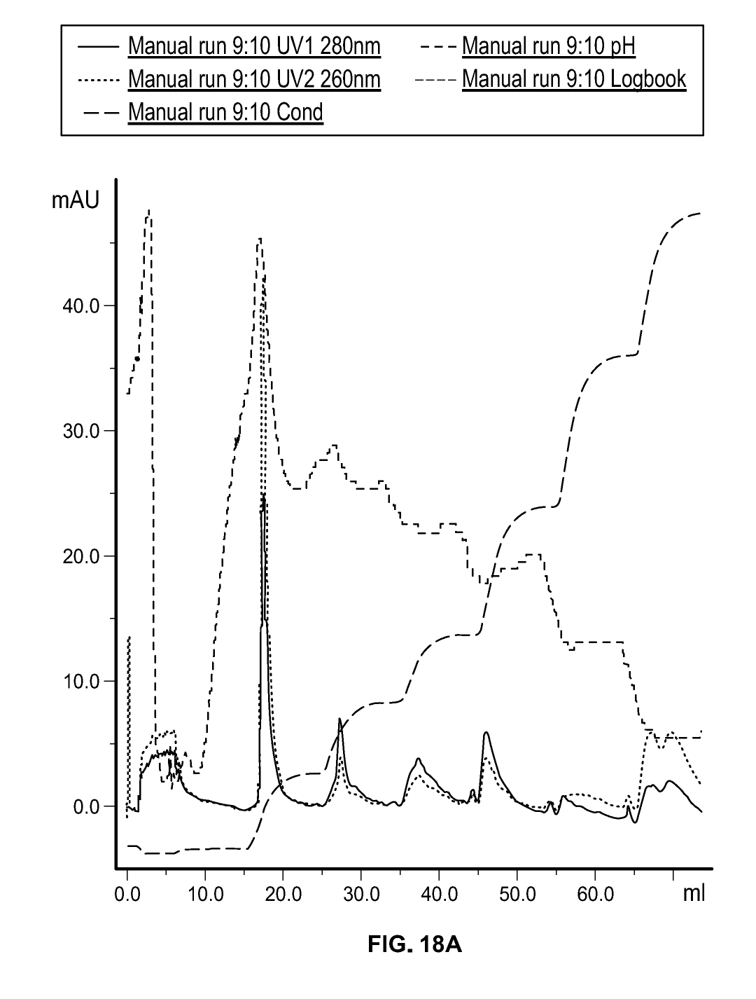

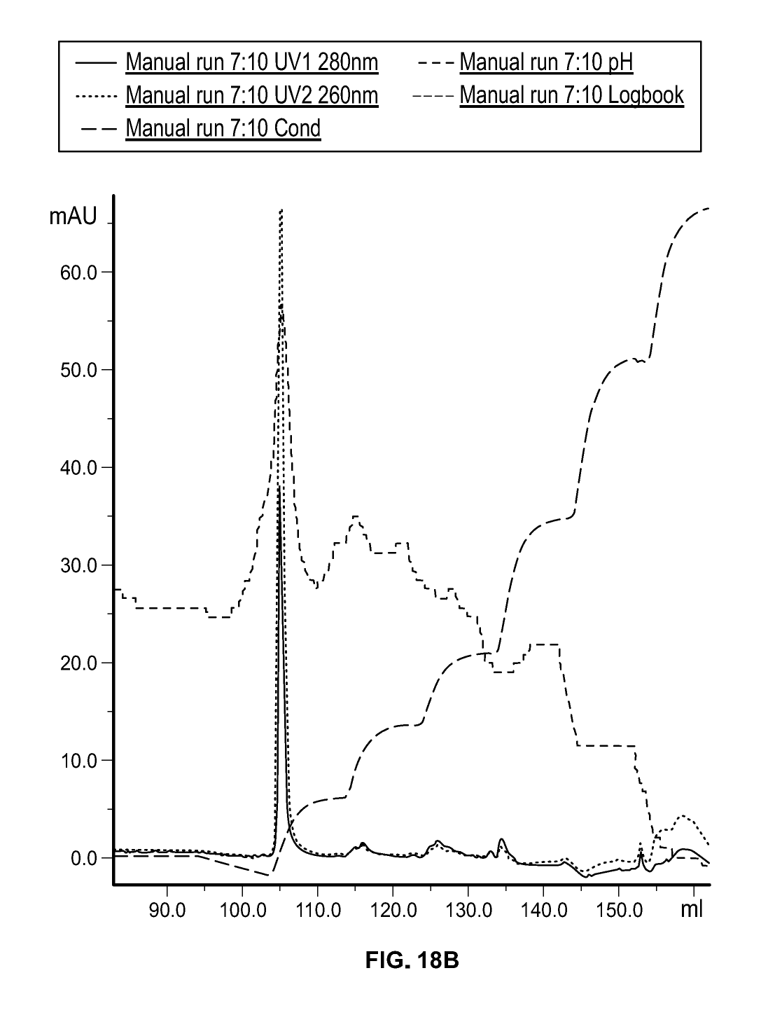

Next, pH- and salt-based elutions for anion exchange chromatography were compared. FIG. 18A shows the elution profile of a pH-based elution of the anion exchange substrate using pH 8.0 phosphate buffer. FIG. 18B shows the elution profile of a NaCl-based elution of the anion exchange substrate using NaCl in pH 8.0 phosphate buffer. These results demonstrate that better purity was achieved using NaCl elution, as compared with pH elution. Both elutions led to >100% yield. pH elution would not require dilution before the anion exchange step, whereas NaCl would require a 10× dilution factor. However, NaCl elution led to higher purity than pH elution. Therefore each type of elution would have benefits, and the desired type may depend upon purity requirements for downstream processing and scaling up.

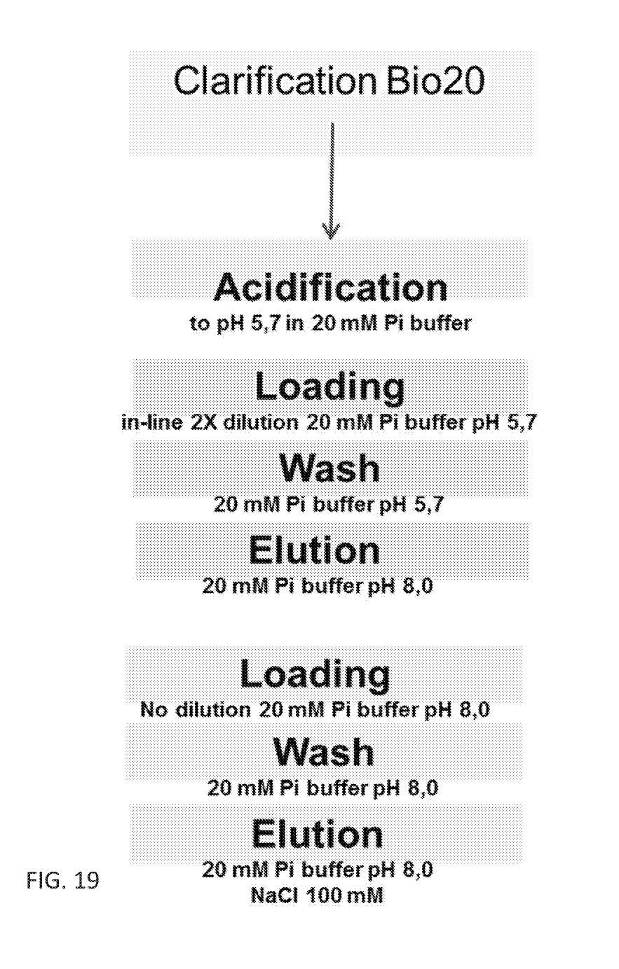

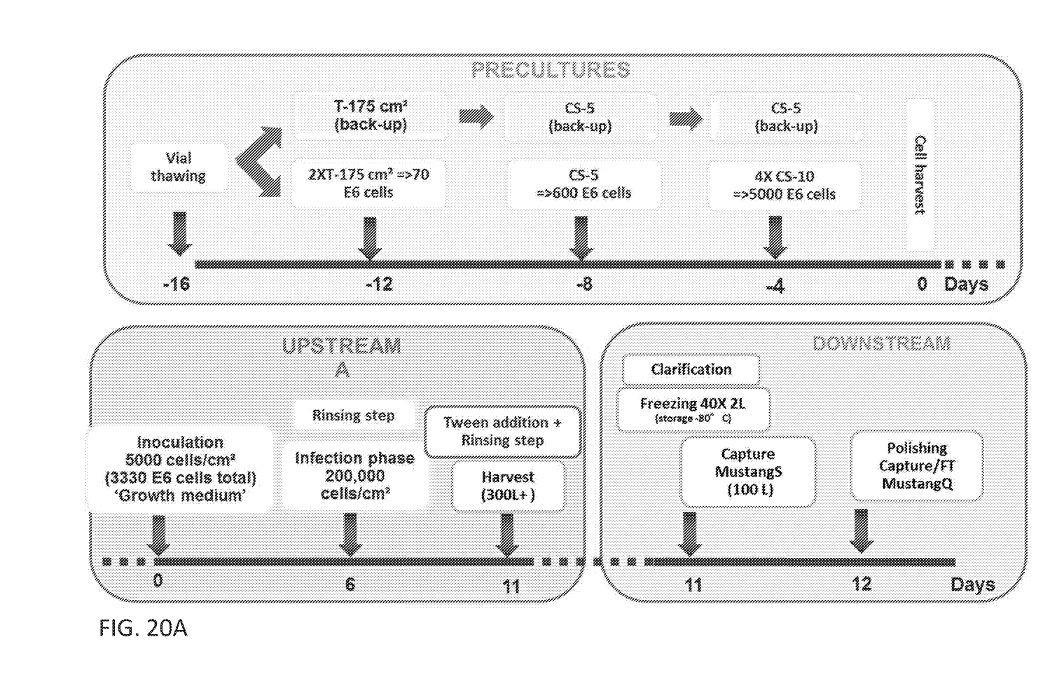

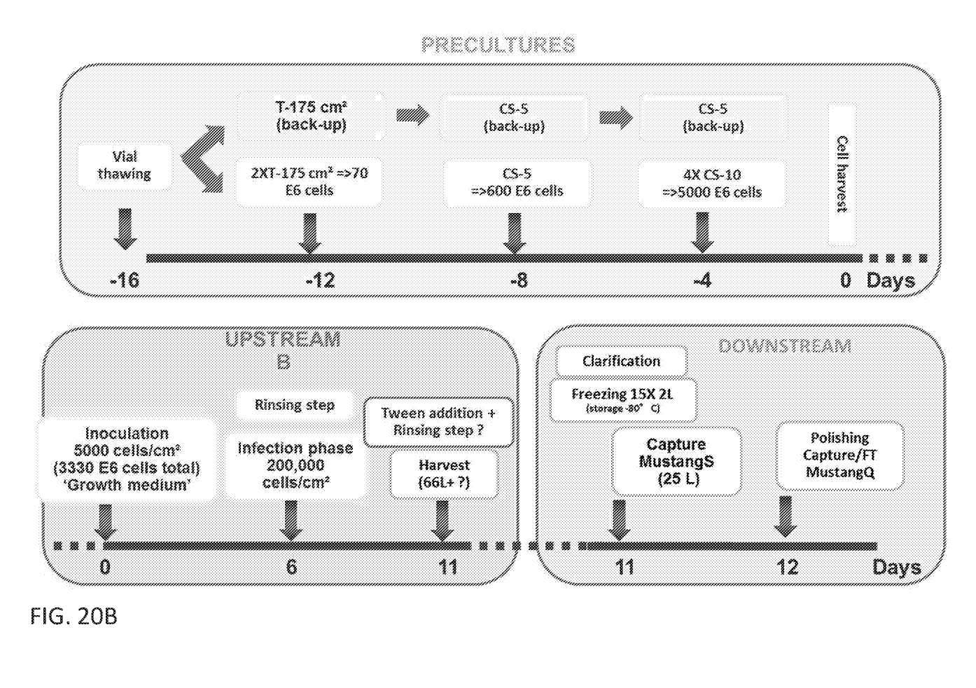

In summary, a diagram of an exemplary downstream processing flow for scaling up virus production is provided in FIG. 19. Two exemplary flowcharts detailing the entire production process are provided in FIGS. 20A & 20B.

### Example 10: Improvement of Process Parameters for Virus Production in Pilot Scale Process

A pilot-scale process run was performed for virus production, harvest, and purification using an iCELLis® 500/66 m² system. This scaled-up system provides a 70-fold increase in bioreactor volume as compared to the iCELLis® NANO (70 L vs. 1 L).

Results

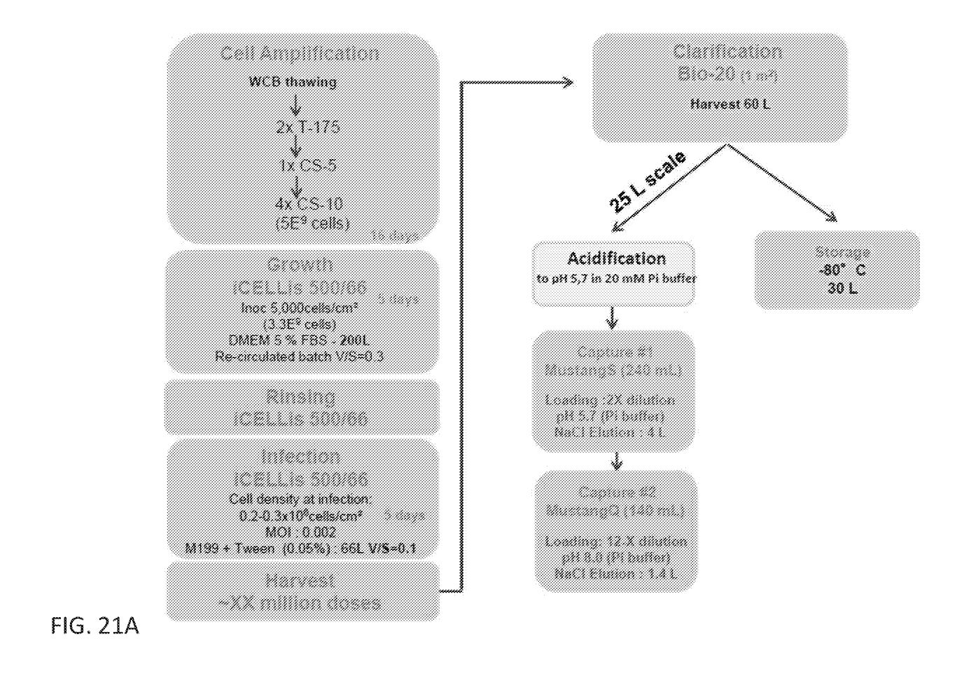

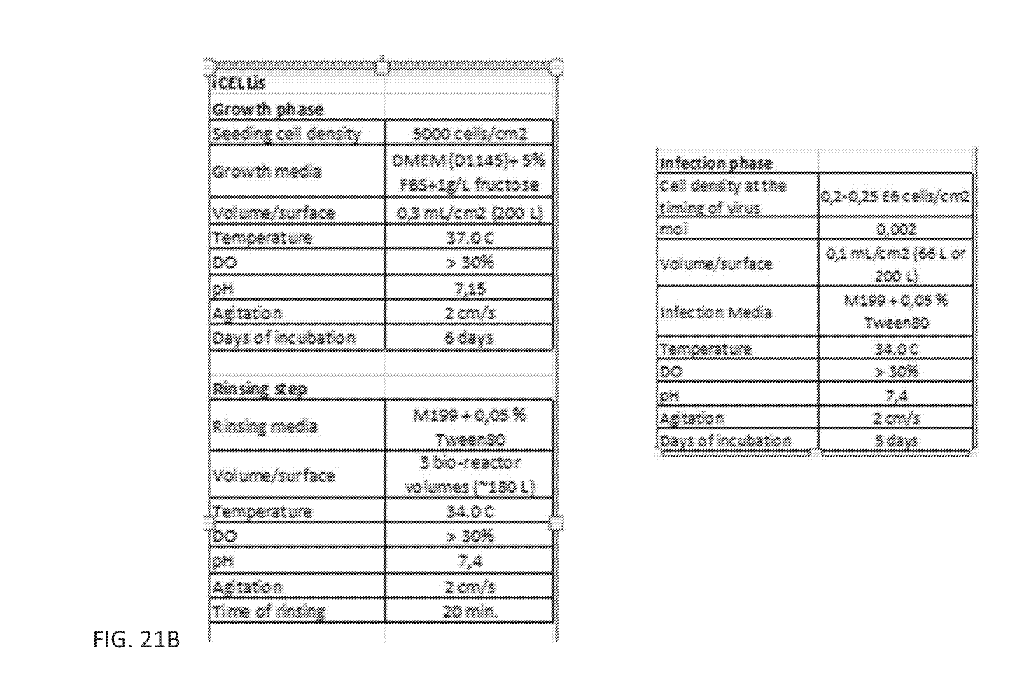

A full process flowchart for virus production using an iCELLis® 500/66 m² system, with subsequent harvest and downstream processes steps, is shown in FIG. 21A. Optimized parameters for upstream processing using an iCELLis® 500/66 m² system are shown in FIG. 21B.

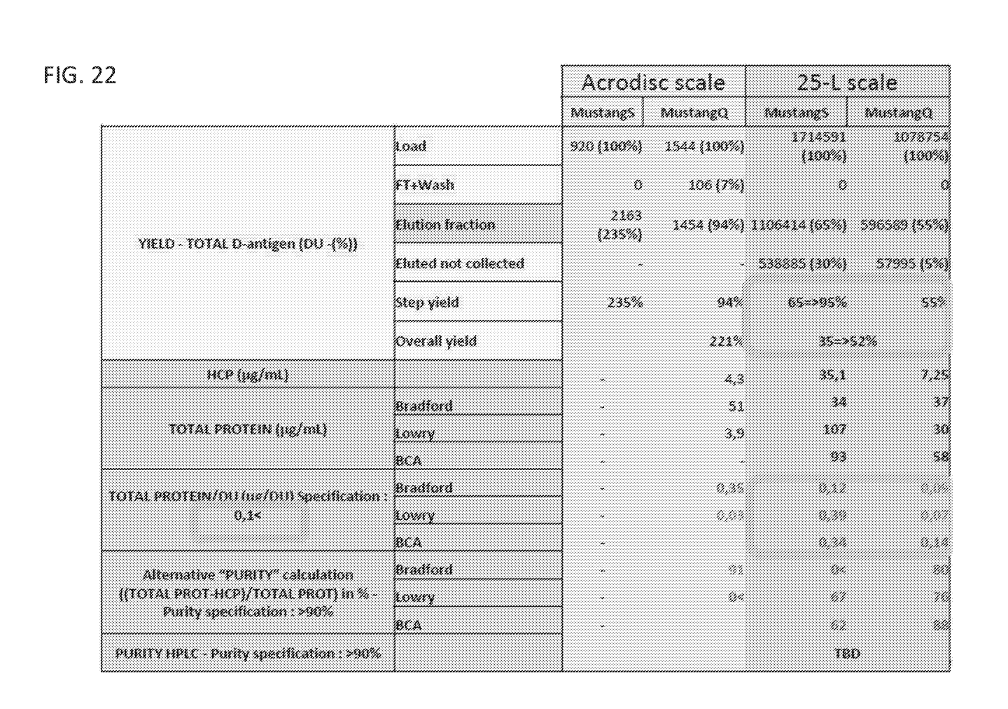

The process shown in FIGS. 21A & 21B was conducted at a 25 L scale. The results of this process were analyzed and compared with a smaller AcroDisc® scale (FIG. 22). As highlighted in FIG. 22, and with respect to D-antigen yield, two peaks were observed in the Mustang-S elute step with a step-wise gradient procedure. One fraction containing only the first peak was applied to Mustang-Q. In this situation, the final D-antigen yield was 35%. However, without wishing to be bound to theory, it is thought that the final yield may increase to 52% if the virus suspension including both peaks were applied to the Mustang-Q. With respect to total protein/DU, the total value of final purified virus met specification, and the total protein concentration values were different among the three different procedures.

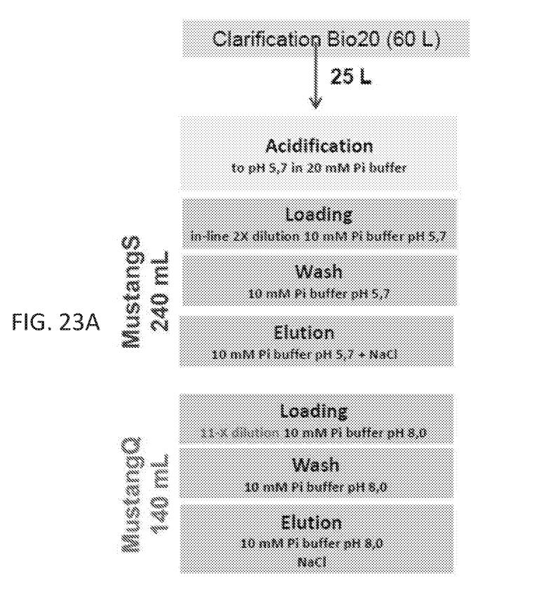

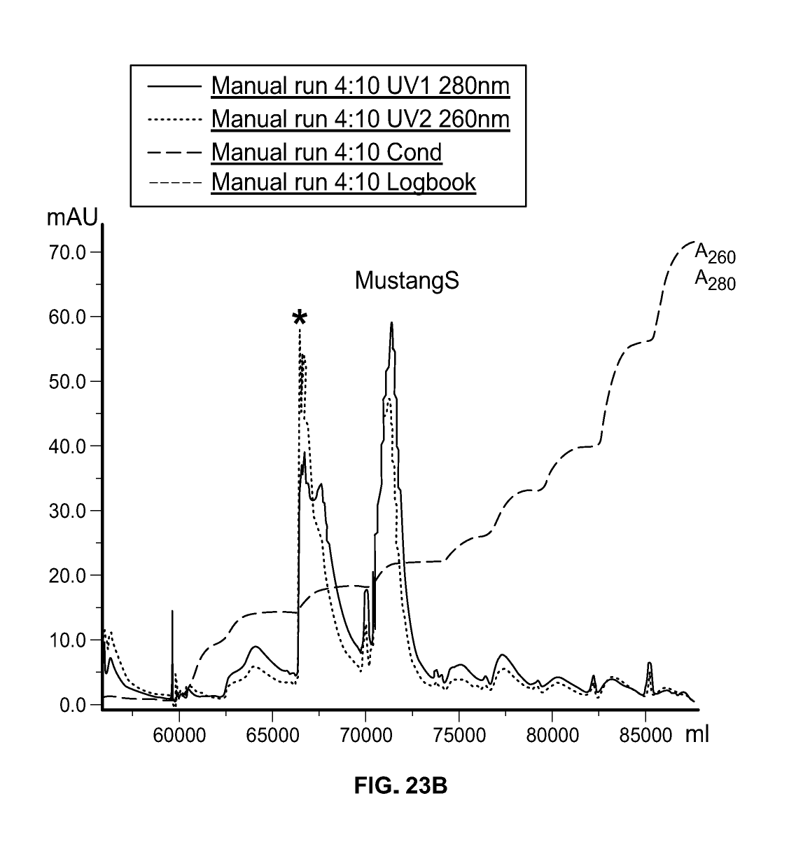

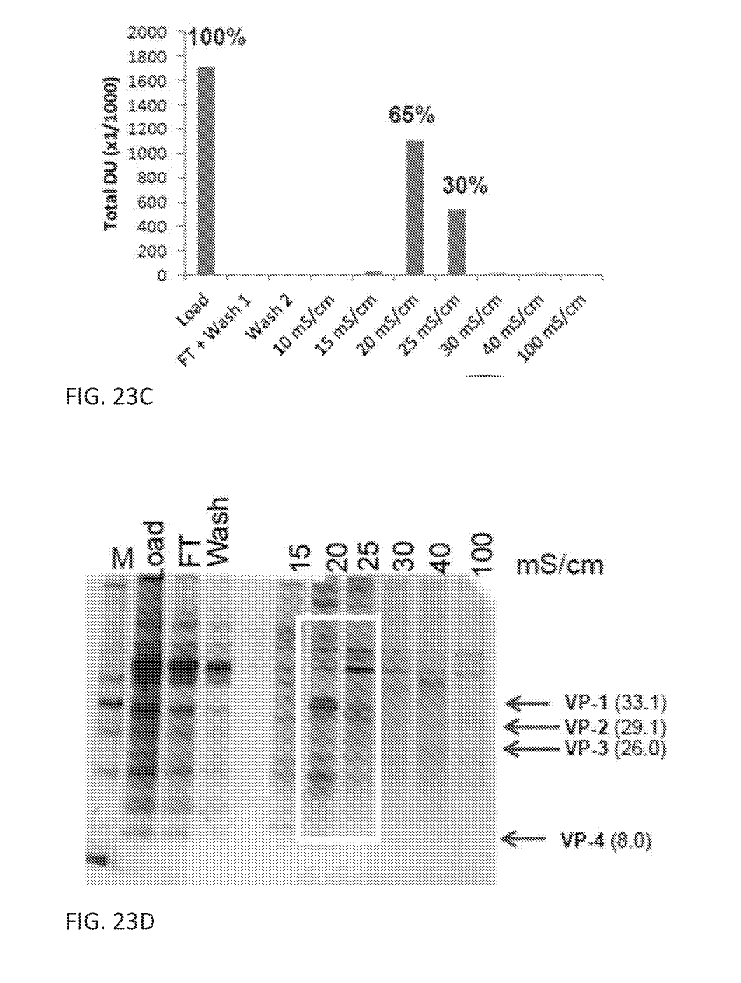

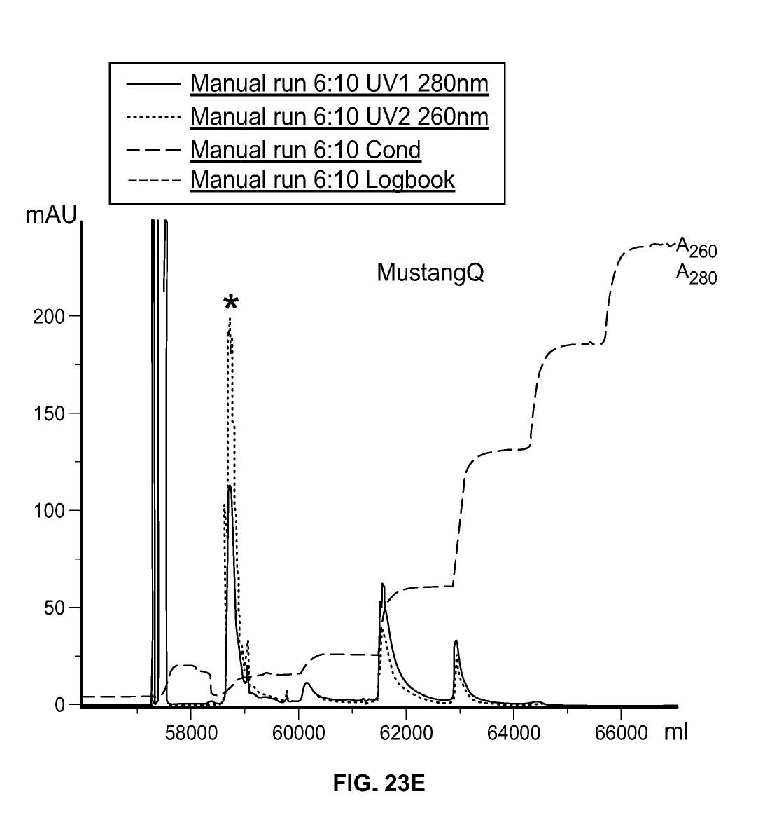

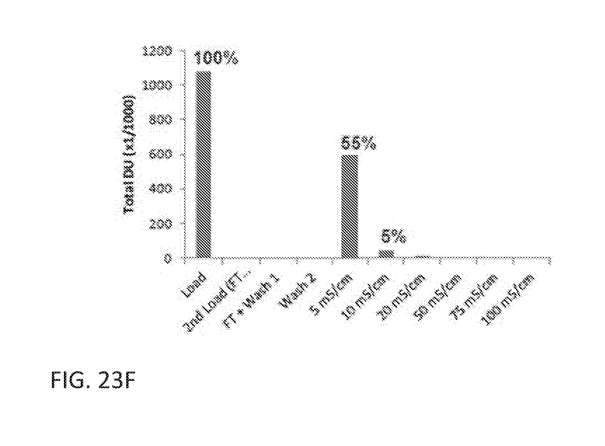

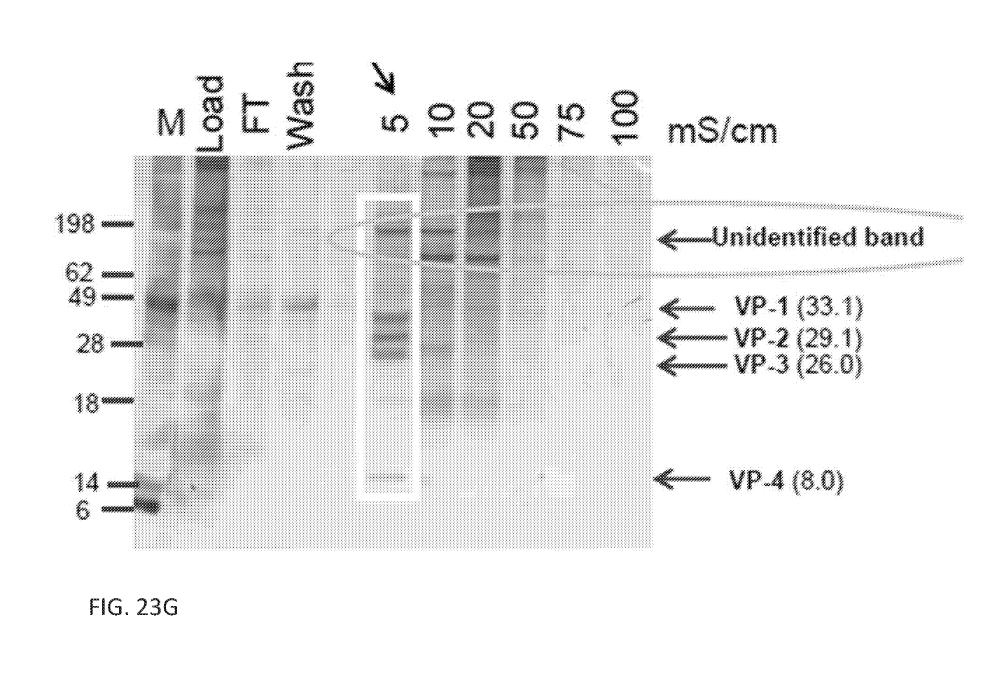

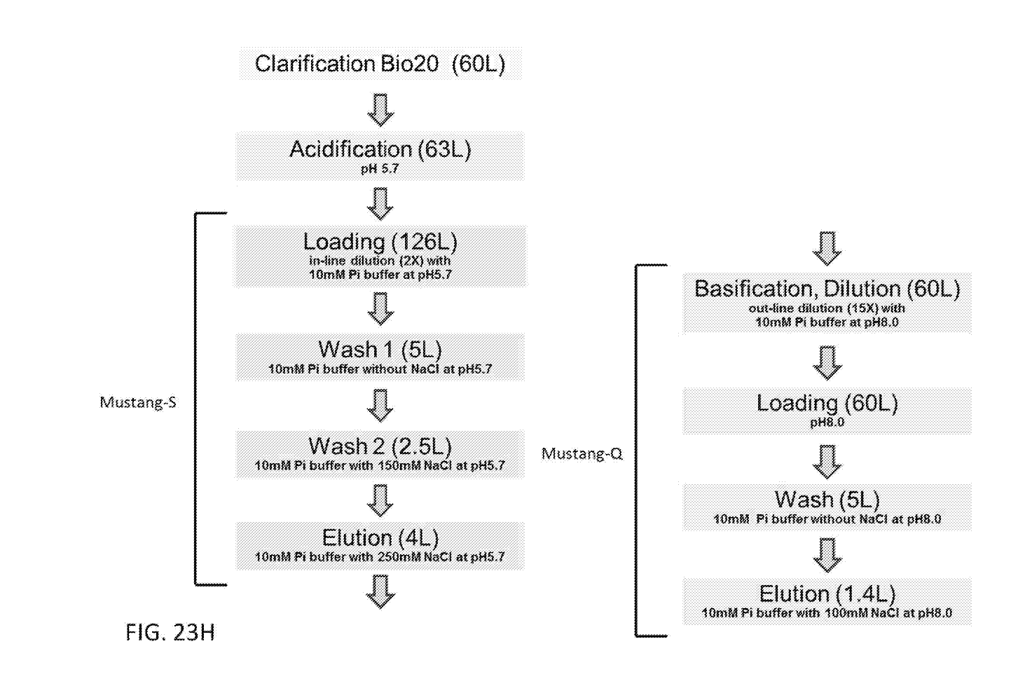

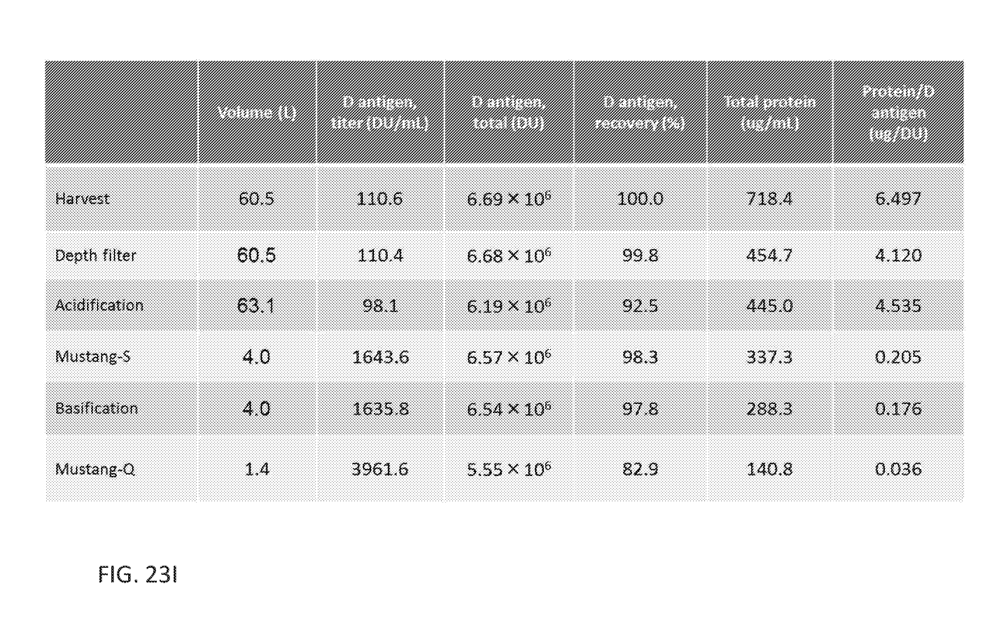

To investigate potential sources for low yield, downstream processing steps were next examined. A summary of downstream processing steps is shown as a flow diagram in FIG. 23A. As shown in FIGS. 23B & 23C, elution at two different conductivities was observed (20 and 25 mS/cm) during cation exchange chromatography. These results were confirmed by the detection of protein bands corresponding to VP1, VP2, and VP3 from the 20 and 25 mS/cm elutions (FIG. 23D). VP4 was not detected.

Next, the elution profiles from anion exchange chromatography were determined (FIG. 23E). As shown in FIG. 23F, a lack of virus recovery in the second load, flow-through and first wash, and second wash steps may indicate a loss of ˜45%. Detection of proteins in various anion exchange elutions demonstrated the presence of an additional, unidentified band migrating a higher molecular weight than VP1, VP2, and VP3 (FIG. 23G).

Because of these results, additional experiments are undertaken to optimize downstream processing and increase purity at larger scale. Without wishing to be bound to theory, it is thought that the elution profiles and purity observed as described above may be driven by unspecified virus/protein interactions. Weakening these interactions is thought to potentially restore a more expected elution profile and provide stronger virus/membrane interactions, leading to elution of a single peak and better purity.

Improving Purity

In one aspect, a higher (e.g., 5-fold higher) polyosorbate concentration (e.g., TWEEN®-80) is used during chromatography loading, wash, and elution.

In another aspect, a higher conductivity (e.g., 10-15 mS/cm) is used during loading.

In another aspect, the contaminating band shown in FIG. 23G is identified (e.g., using silver-staining/MS analysis). If the band reflects BSA, an improved iCELLis rinsing step is employed.

Achieving a Single Cation Exchange Elution Peak

First, an iCELLis® 500/66 m² system harvest is purified using the smaller AcroDisc® scale to determine whether a two-peak elution is observed.

In another aspect, a higher (e.g., 5-fold higher) polysorbate concentration (e.g., TWEEN®-80 concentration) is used during chromatography loading, wash, and elution. For example, the following concentrations of polysorbate (e.g., TWEEN®-80) are tested: 0% (as a negative control), 0.05%, 0.1%, 0.25%, and 0.5%.

In another aspect, a higher conductivity (e.g., 10-15 mS/cm) is used during loading.

Increasing Recovery from Anion Exchange Chromatography

First, an iCELLis® 500/66 m² system harvest is purified using the smaller AcroDisc® scale to determine whether a similar recovery is observed.

In another aspect, quantification of 25 L scale is re-checked to determine whether the lower observed yield (e.g., as compared to AcroDisc® scale) is due to quantification error.

Sizing Chromatography Steps

Maximum loading capacities of the cation exchange column (e.g., a Mustang S chromatography membrane) and anion exchange column (e.g., a Mustang Q chromatography membrane) are determined. For example, 125 mL/mLMV, 250 mL/mLMV, 500 mL/mLMV of harvest are loaded at 10 mL Mustang membrane scale.

Improving Anion Exchange Chromatography

To improve capture using anion exchange chromatography, multiple approaches are used. The final pH after dilution was 7.4, not 8.0. In one aspect, buffer capacity is increased to reach pH 8.0 during dilution. In another aspect, the impact of pH 7.4 on yield is determined. The acceptable pH range for Mustang-Q load may be based, e.g., on manufacturing records.

In another aspect, 15 mM and 20 mM phosphate buffers are used for dilution and examined for their effects on conductivity, dilution factor, and yield.

In another aspect, capture on anion exchange membrane at pH 7.5 is tested to evaluate the potential use of a lower pH.

A full production run was conducted using S2 virus. The downstream process parameters are depicted in FIG. 23H. The results, including volume: D-antigen titer, total amount, and recovery; total protein; and protein/D-antigen ratio for each step of the downstream process are shown in FIG. 23I.

### Example 11: Comparison of Upstream Process Parameters for Virus Production in Pilot Scale Process

Additional pilot-scale process runs were performed for virus production, harvest, and purification using an iCELLis® 500/66 m² system and compared to production using the iCELLis® NANO system. Various upstream process parameters were monitored for potential effects on productivity. Production of multiple poliovirus strains was examined.

Results

Four independent production runs were carried out: two with cells grown in an iCELLis® 500/66 m² system and two with cells grown in an iCELLis® NANO system. FIG. 24A shows various parameters of the cell cultures in each condition (all conditions used strain S2). Importantly, highest D-antigen titer/mL was observed for one of the cultures grown in iCELLis® 500/66 m² system (“iCELLis 500/66 B”), which generated an estimated 39.4M DU, corresponding to 0.55M doses of vaccine from a single run. Three additional runs, two with cells grown in an iCELLis® 500/66 m² system and one with cells grown in an iCELLis® NANO system were also undertaken, as shown in FIGS. 24B & 24C (all conditions used strain S3). These results demonstrated that growth in an iCELLis® 500/66 m² system led to nearly equivalent relative productivity as compared to growth in an iCELLis® NANO system (D-antigen titer at v/s0.3 of 118.9 DU/mL for 500/66 vs. 104.0 DU/mL for NANO).

### Example 12: Improvement of Downstream Process Parameters for Virus Production in Pilot Scale Process

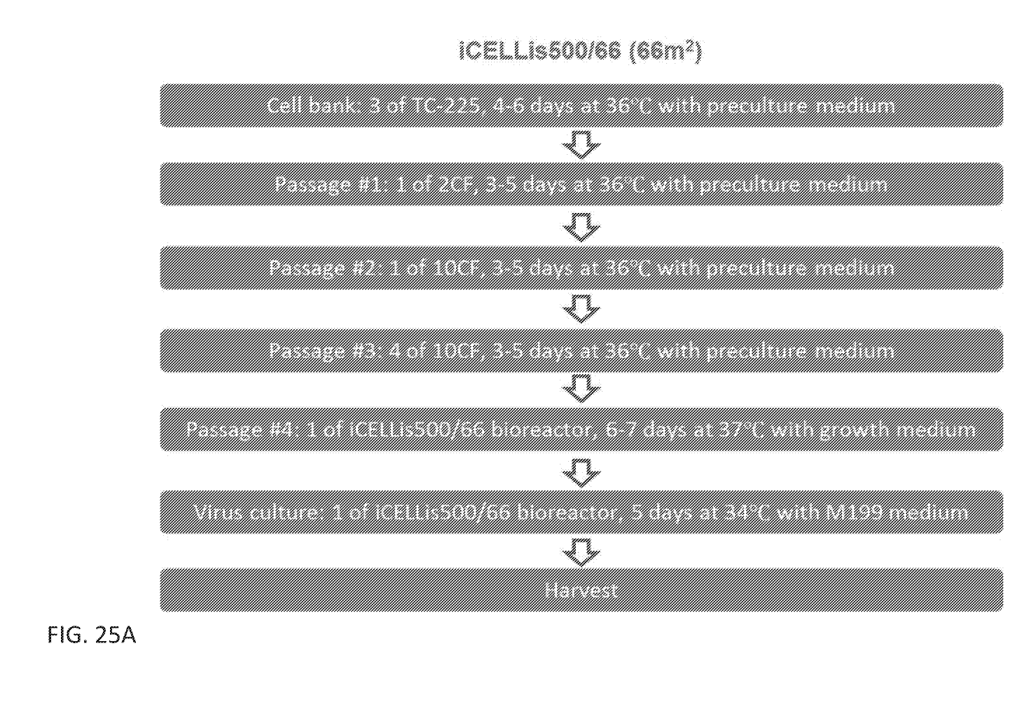

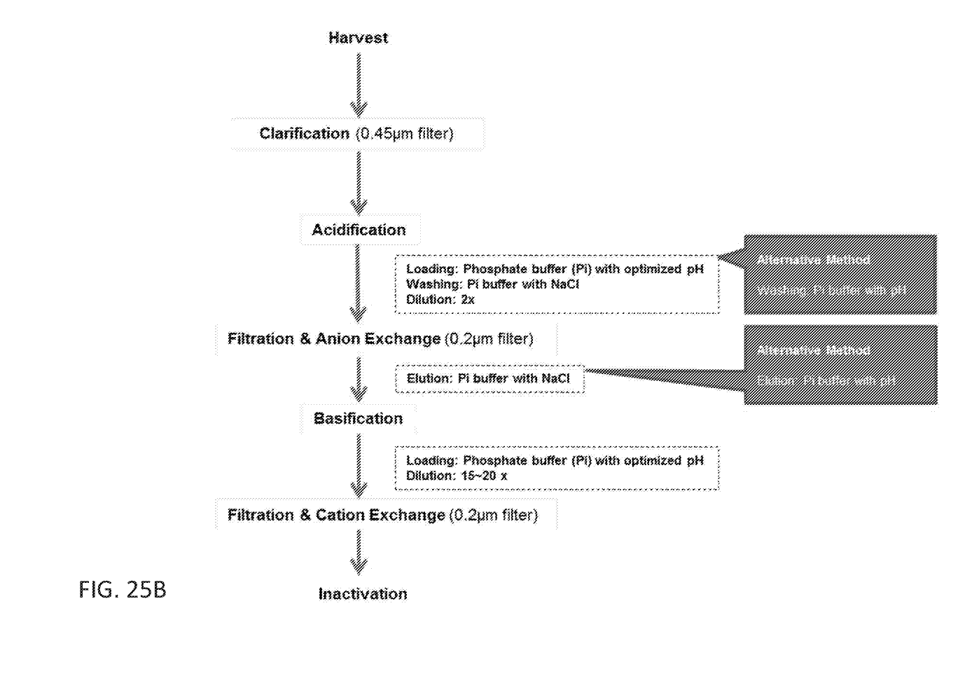

Further improvements to downstream processing steps for virus production were next examined. The upstream processing steps used for these experiments are diagrammed in FIG. 25A.

Results

Exemplary downstream processes for virus harvest and purification are shown in FIG. 25B. One process uses washing and elution of anion exchange chromatography with NaCl buffer, whereas the alternative process uses a pH-based wash and elution using phosphate buffer. The differences between processes are summarized in Tables E and F below.

TABLE E
Downstream process parameters.

| | | | | Alternative DSP |
|---|---|---|---|---|
| | | | | Parameters |
| | | | Current DSP Parameters | (pH Washing and |
| | | | (NaCl Washing and Elution) | Elution) |
| General Setting | | Flow speed | 10 MV/min | 30 MV/min |
| | | Tween80 | 0.05% | 0.05% |
| | | Phosphate conc. | 10 mM | 10 mM |
| Mustang-S | Load | pH | 5.7 | (5.7) |
| | | Dilution factor | 2 | 2 |
| | Wash | Buffer | 10 mM Phosphate Buffer | — |
| | | NaCl conc. | No Data Available | — |
| | | | (0 mM) | |
| | | pH | 5.7 | 6.5 |
| | | Volume | 10MV | 30MV |
| | Elute | NaCl conc. | 250 mM | — |
| | | pH | 5.7 | 8.0 |
| | | Volume of each | 15MV | 30MV |
| | | fraction | | |
| | Assay | D-antigen to choose | Not required | Not required |
| | | fractions | | |

TABLE F
Downstream process parameters (continued).

| | | | Current DSP | Alternative DSP |
|---|---|---|---|---|
| | | | Parameters | Parameters |
| | | | (NaCl Washing and | (pH Washing and |
| | | | Elution) | Elution) |
| Mustang-Q | Load | pH | 8.5 | 8.0 |
| | | Dilution factor | x15 | — |
| | Wash | Buffer | 10 mM Phosphate Buffer | — |
| | | NaCl conc. | — | — |
| | | pH | 8.5 | 8.0 |
| | | Volume | 20MV | 30MV |
| | Elute | Gradient | 300 mM | 1M NaCl |
| | | | | 10% stepwise |
| | | pH | 8.5 | 8.0 |
| | | Volume of each fraction | 15MV | 20MV |

These parameters were applied to S1 and S3 virus, as shown in Tables G and H below.

TABLE G
Results using S1 virus.

| | Total DU | | |
|---|---|---|---|
| | Load | FT + wash | Elution fraction |
| MSTG S | 2664.61 Du | 2.236 Du | 2776.95 Du |
| | (100%) | (0.08%%) | (Approx. 100%) |
| MSTG Q | 2864.55 Du | BDL | 2595.65 Du |
| | (100%) | | (90.61%) |

TABLE H
Results using S3 virus.

| | Total DU | | |
|---|---|---|---|
| | Load | FT + wash | Elution fraction |
| MSTG S | 16152.8 Du | BDL | 13053.20 Du |
| | (100%) | | (80.81%) |
| MSTG Q | 14161.2 Du | BDL | 1787.25 Du |
| | (100%) | | (12.62%) |

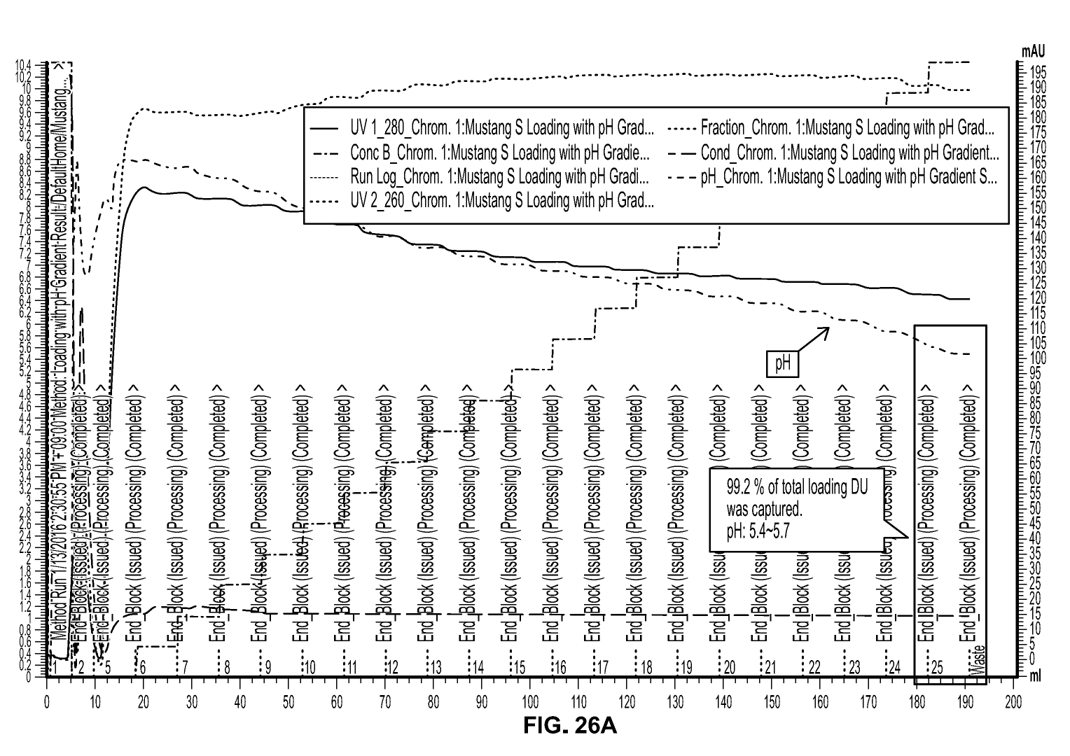

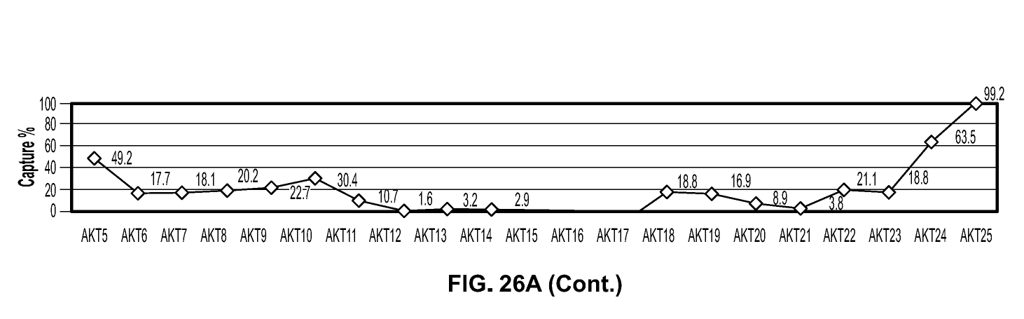

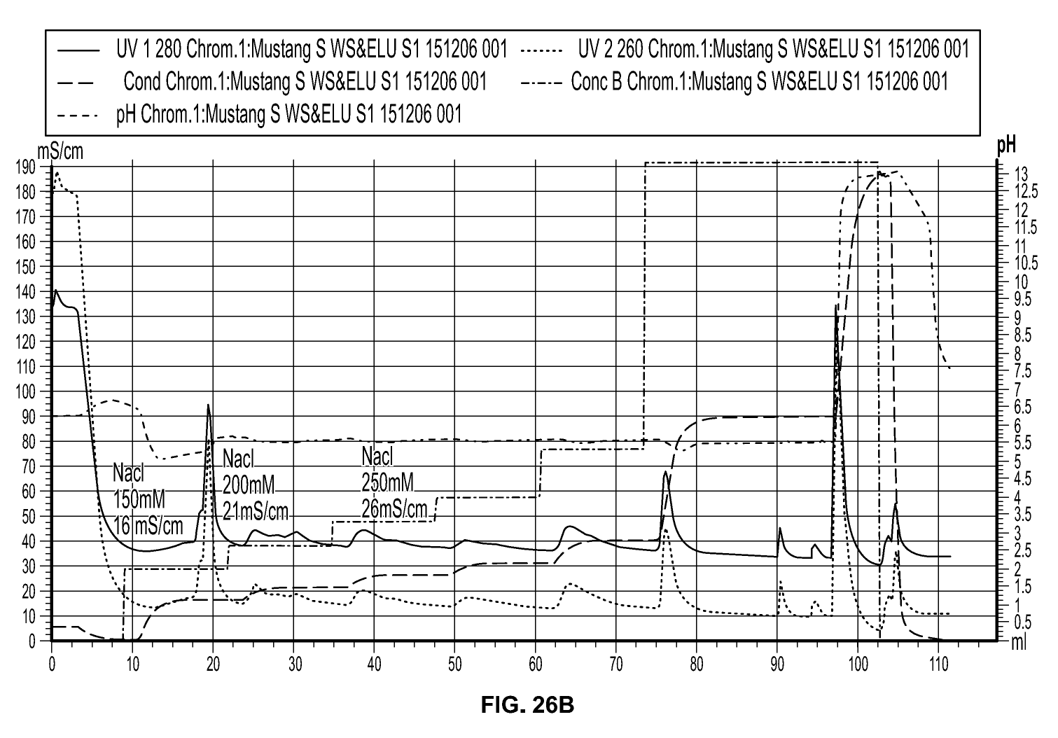

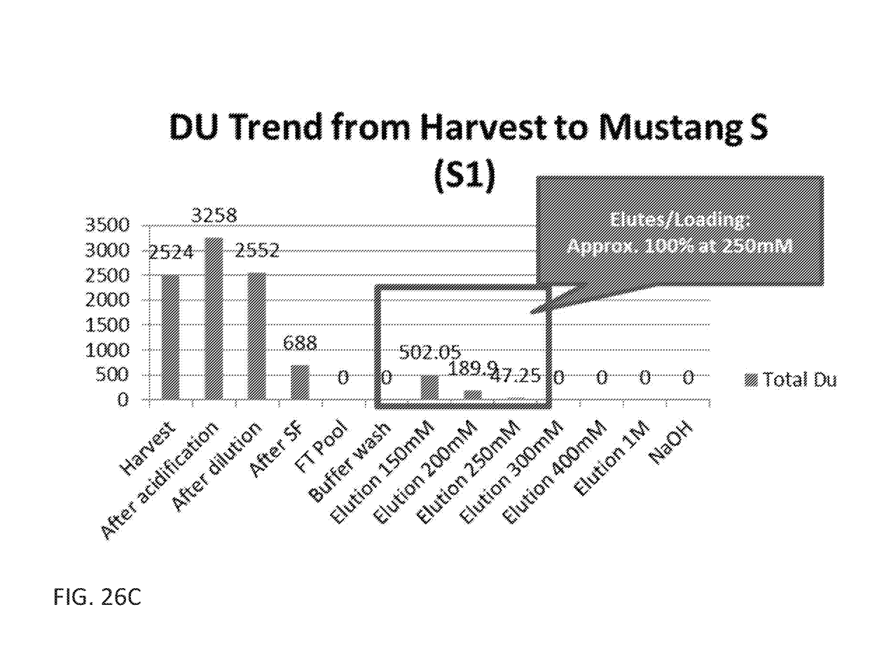

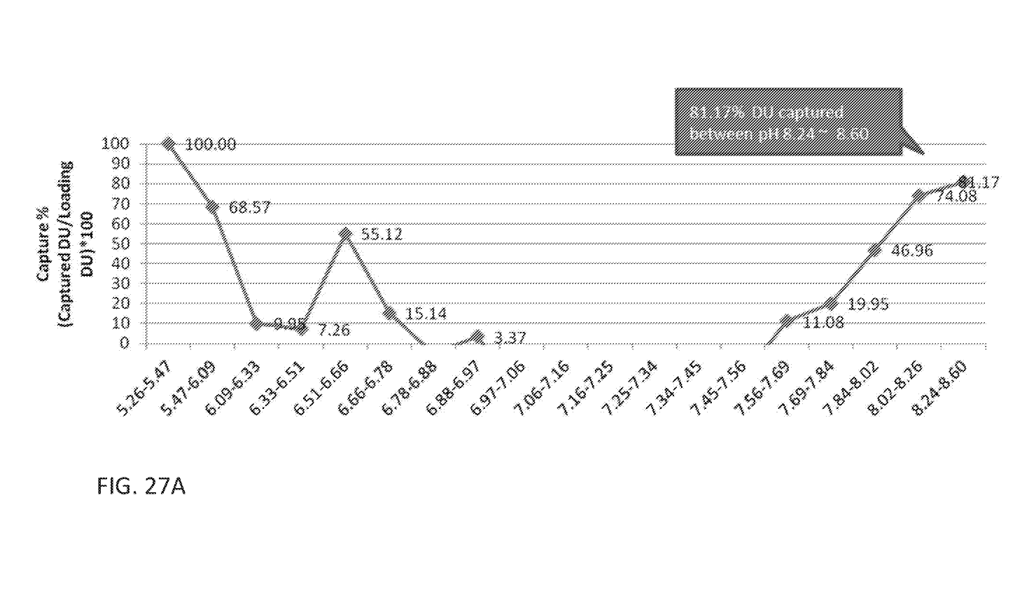

pH for cation exchange membrane loading was examined using strain S1. As shown in FIG. 26A, 99.2% of total virus (DU) was captured between pH 5.4 and 5.7. NaCl elution from cation exchange membrane was also examined using strain S1. Approximately 100% of virus was observed to elute at 250 mM NaCl (FIGS. 26B & 26C). These results indicate that for anion exchange chromatography using the S1 strain, cation exchange loading was most effective at pH 5.7, and 10 mM phosphate buffer with 250 mM NaCl was most effective for elution.

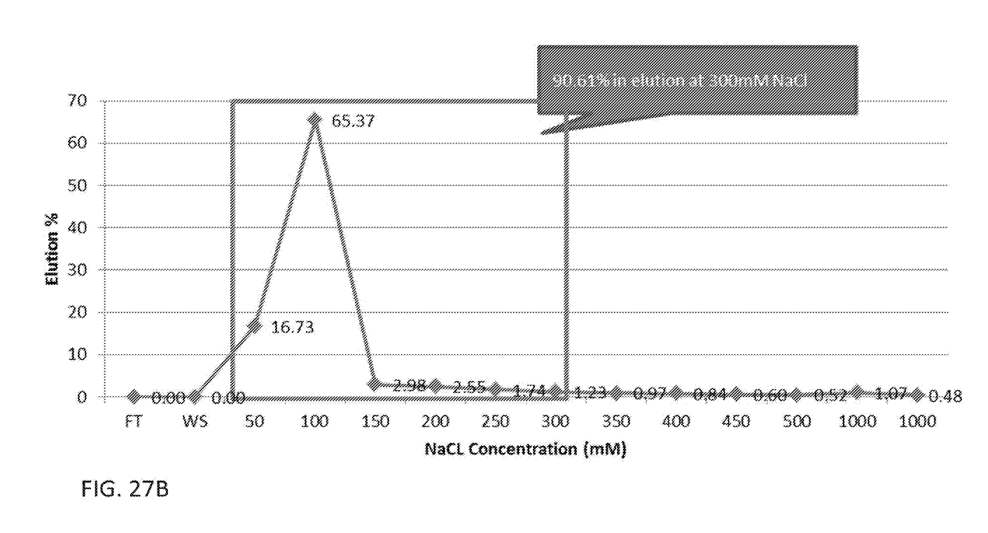

pH for anion exchange membrane loading was next examined using strain S1. Sample was diluted 2× and aliquoted to 2 units (pH 4.0 and 10.0). Loading conductivity was 3.88 mS/cm, and 0.05% TWEEN®-80 was included. As shown in FIG. 27A, 81.17% of total virus (DU) was captured between pH 8.24 and 8.60. NaCl elution from anion exchange membrane was also examined using strain S1. 2864.55 DU virus was purified from a loading pH 8.5, and 0.005% TWEEN®-80 was included. Approximately 90.61% of virus was observed to elute at 300 mM NaCl (FIG. 27B). No DU was observed in flow-through and wash. These results indicate that for anion exchange chromatography using the S1 strain, 10 mM phosphate buffer with 300 mM NaCl elution was most effective.

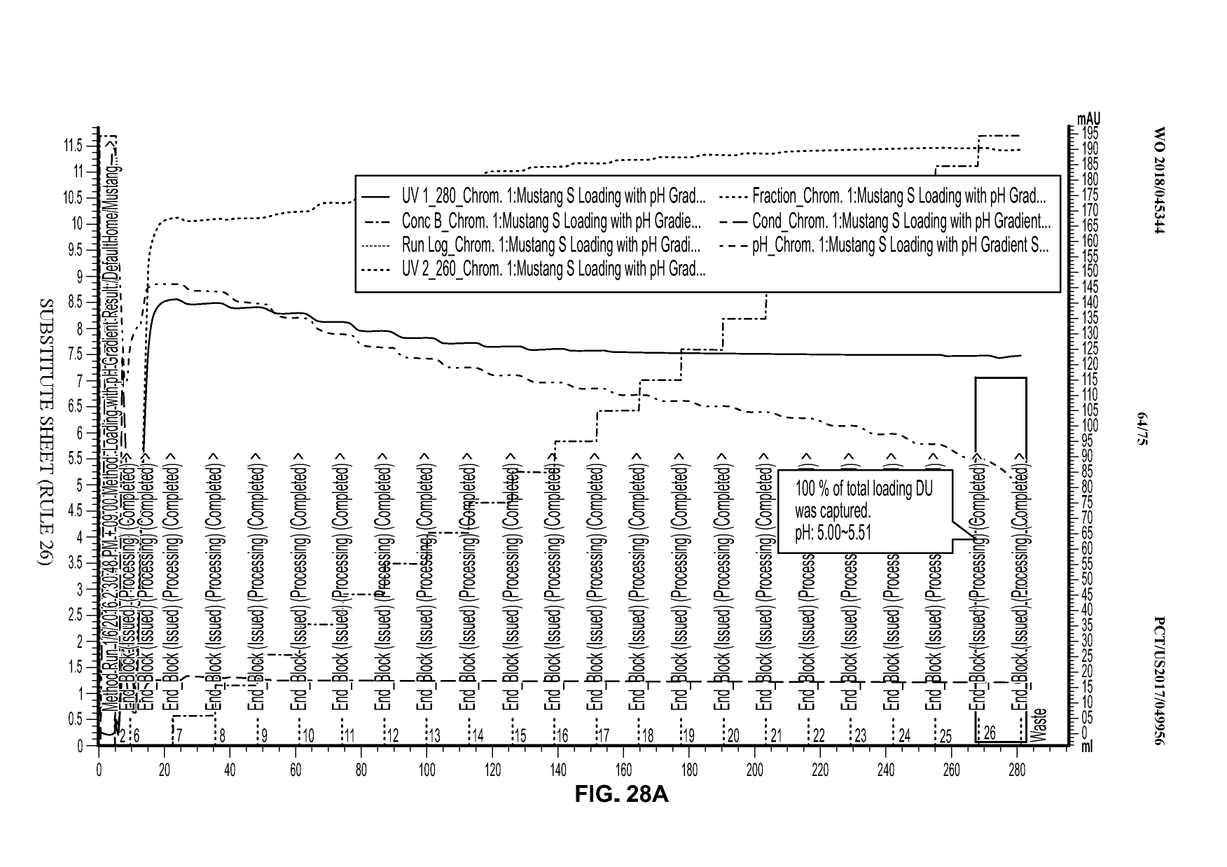

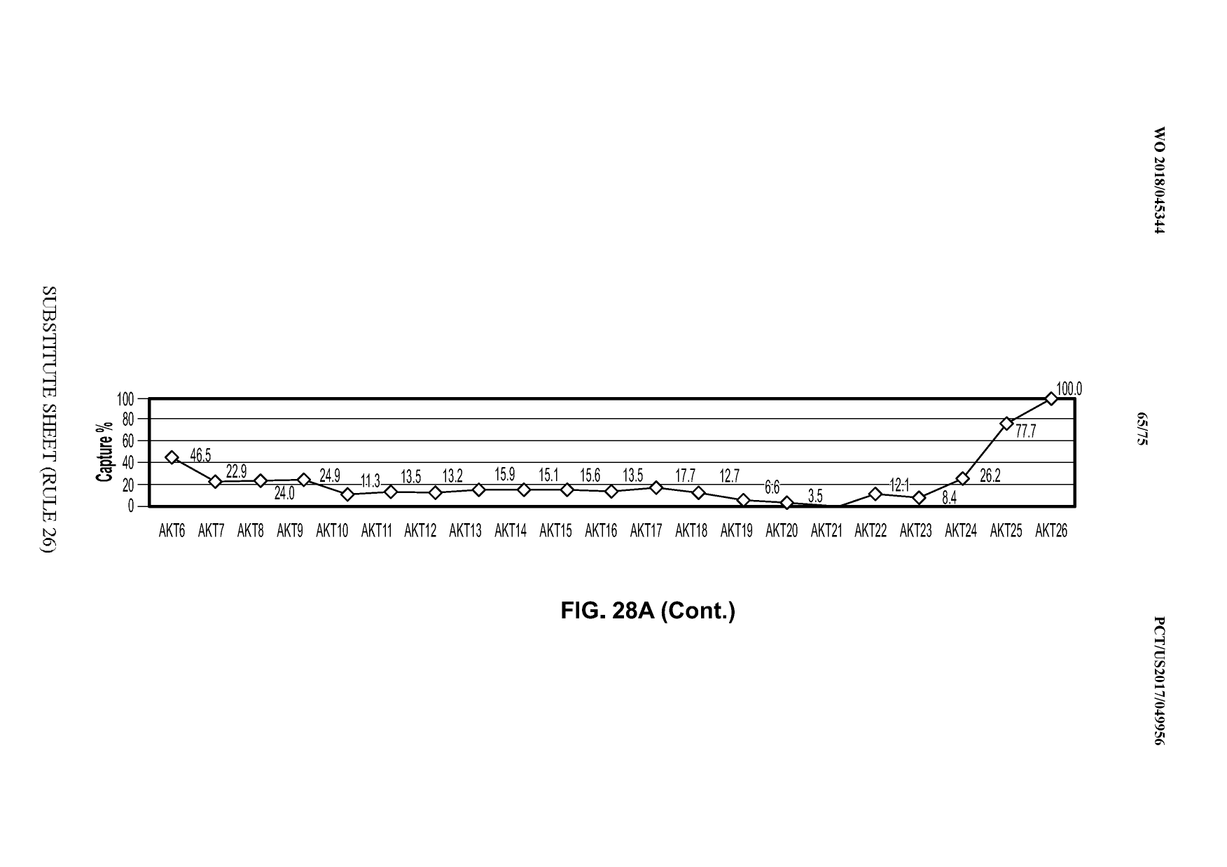

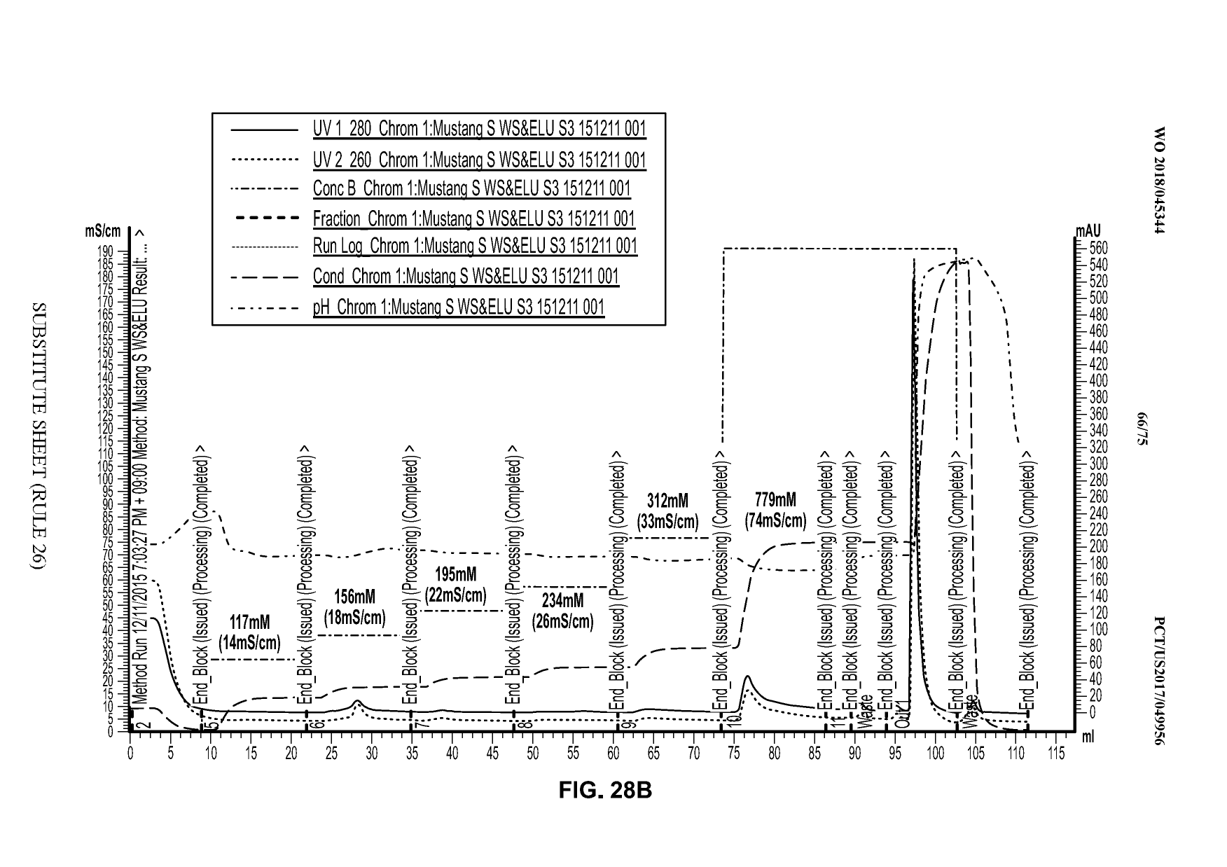

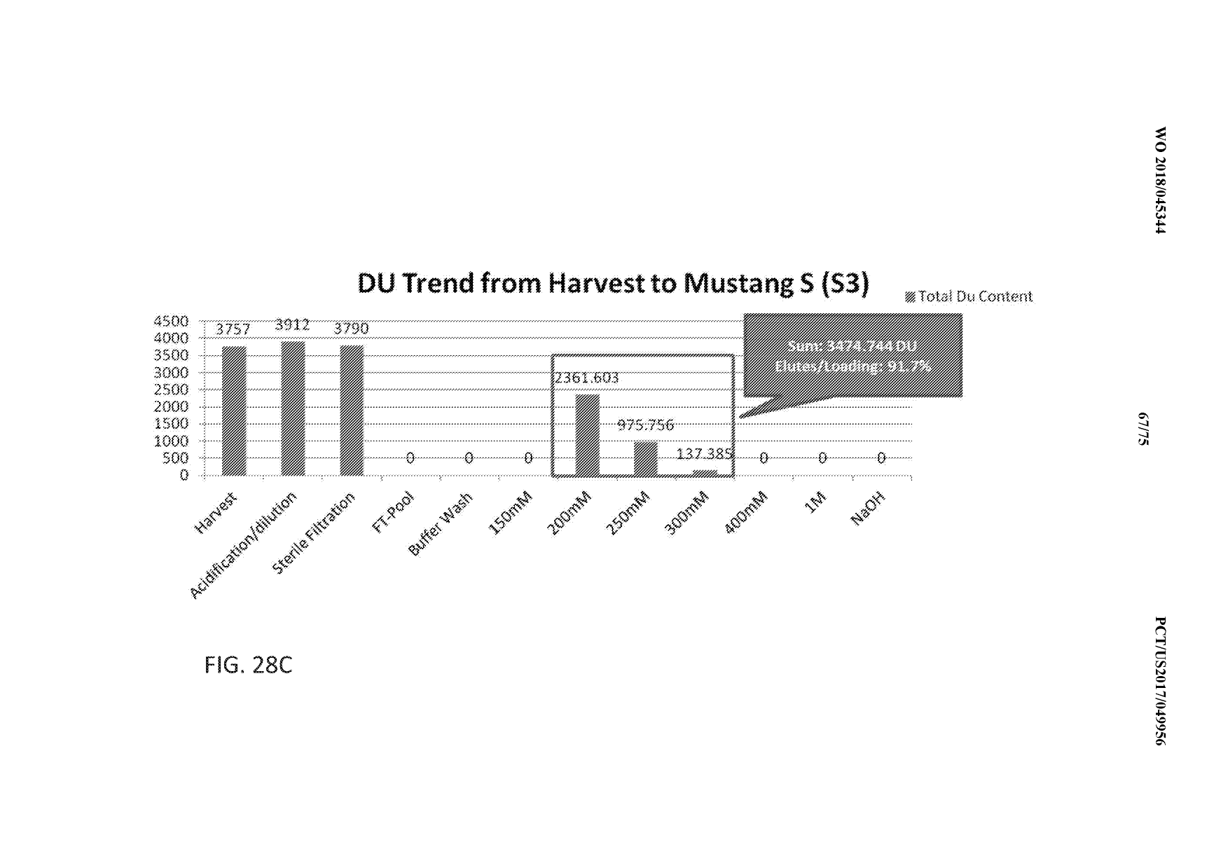

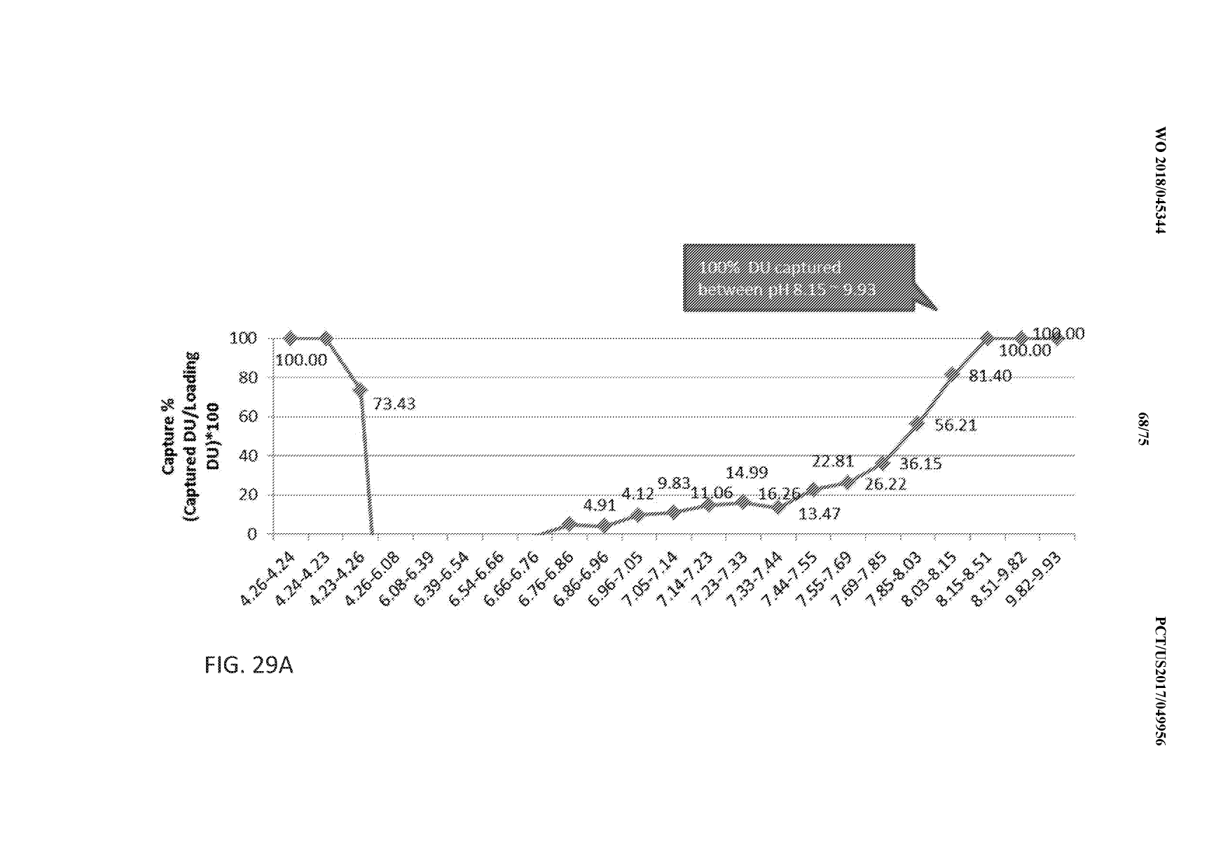

Anion and cation exchange steps were also tested using strain S3. For pH loading onto cation exchange membrane, as shown in FIG. 28A, 100% of total virus (DU) was captured between pH 5.00 and 5.51. NaCl elution from cation exchange membrane was also examined using strain S3. Approximately 91.7% of virus was observed to elute at 200 mM to 300 mM NaCl (FIGS. 28B & 28C). These results indicate that loading the cation exchange membrane using the S3 strain was most effective at pH 5.0, and that elution was most effective with 10 mM phosphate buffer and 300 mM NaCl.

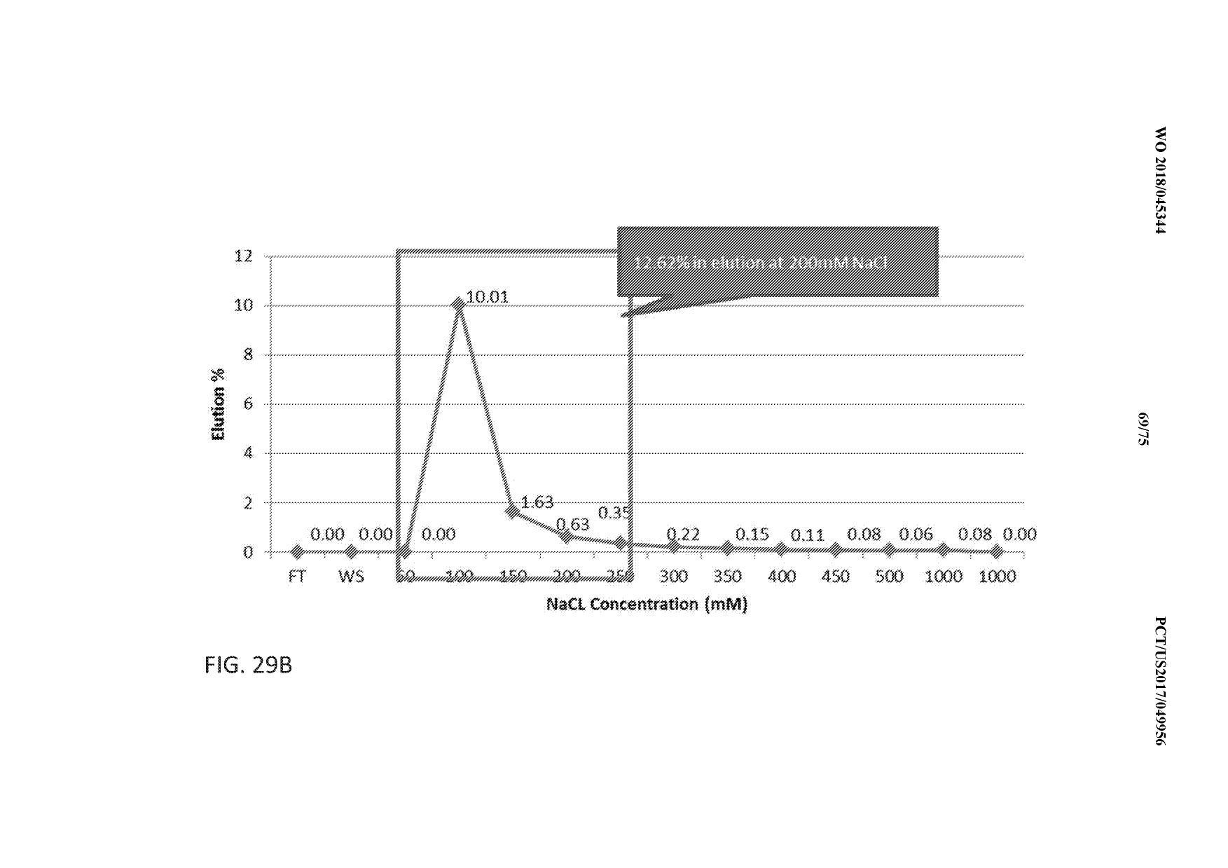

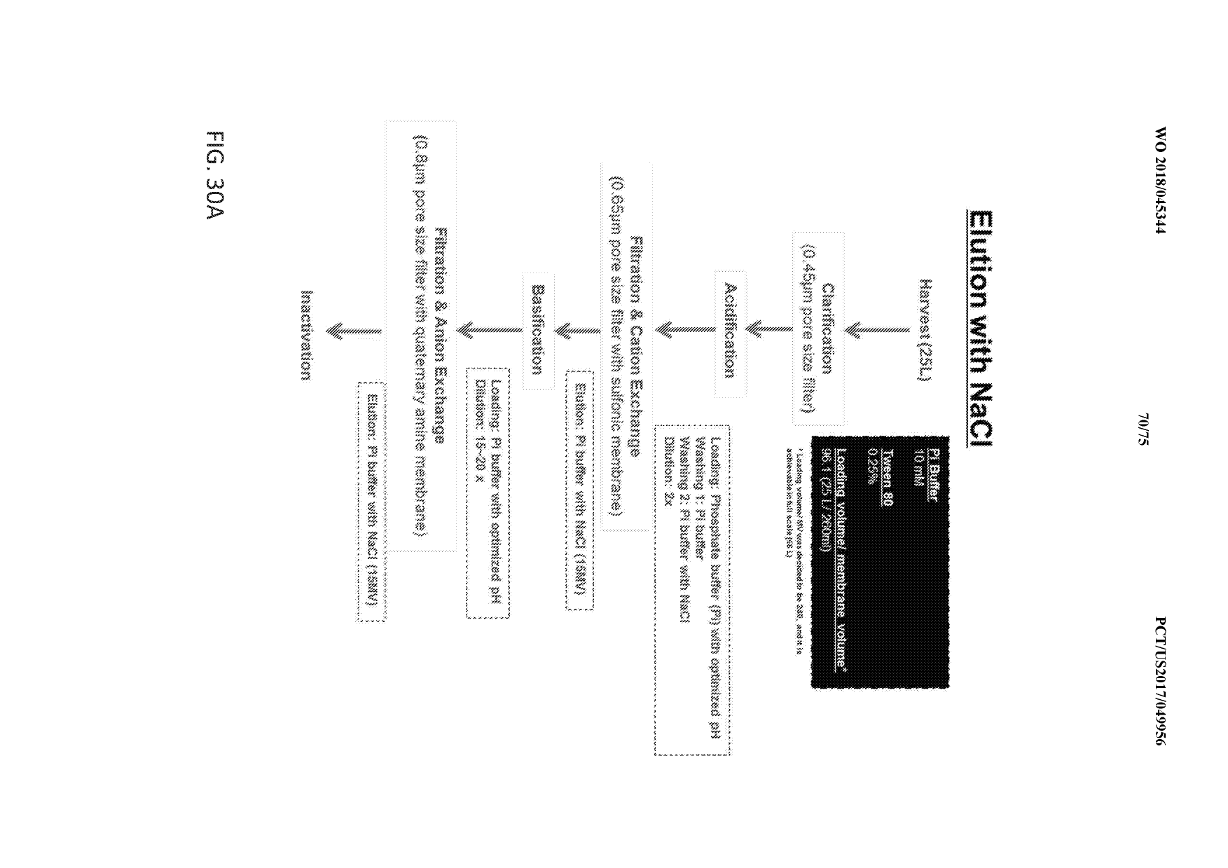

pH for anion exchange membrane loading was next examined using strain S3. Sample was diluted 2× and aliquoted to 2 units (pH 4.0 and 10.0). Loading conductivity was 3.88 mS/cm, and 0.05% TWEEN®-80 was included. As shown in FIG. 29A, 100% of total virus (DU) was captured between pH 8.15 and 9.93. NaCl elution from anion exchange membrane was also examined using strain S3. 14161.2 DU virus was purified from a loading pH 8.5. Approximately 12.62% of virus was observed to elute at 200 mM NaCl (FIG. 29B). No DU was observed in flow-through and wash. These results indicate that for purification of S3 virus, 200 mM NaCl was most effective for elution from the anion exchange membrane. Without wishing to be bound to theory, it is thought that the surface charge difference between S3 and S1/S2 results in more severe parameter settings for anion exchange than cation exchange for the S3 strain.

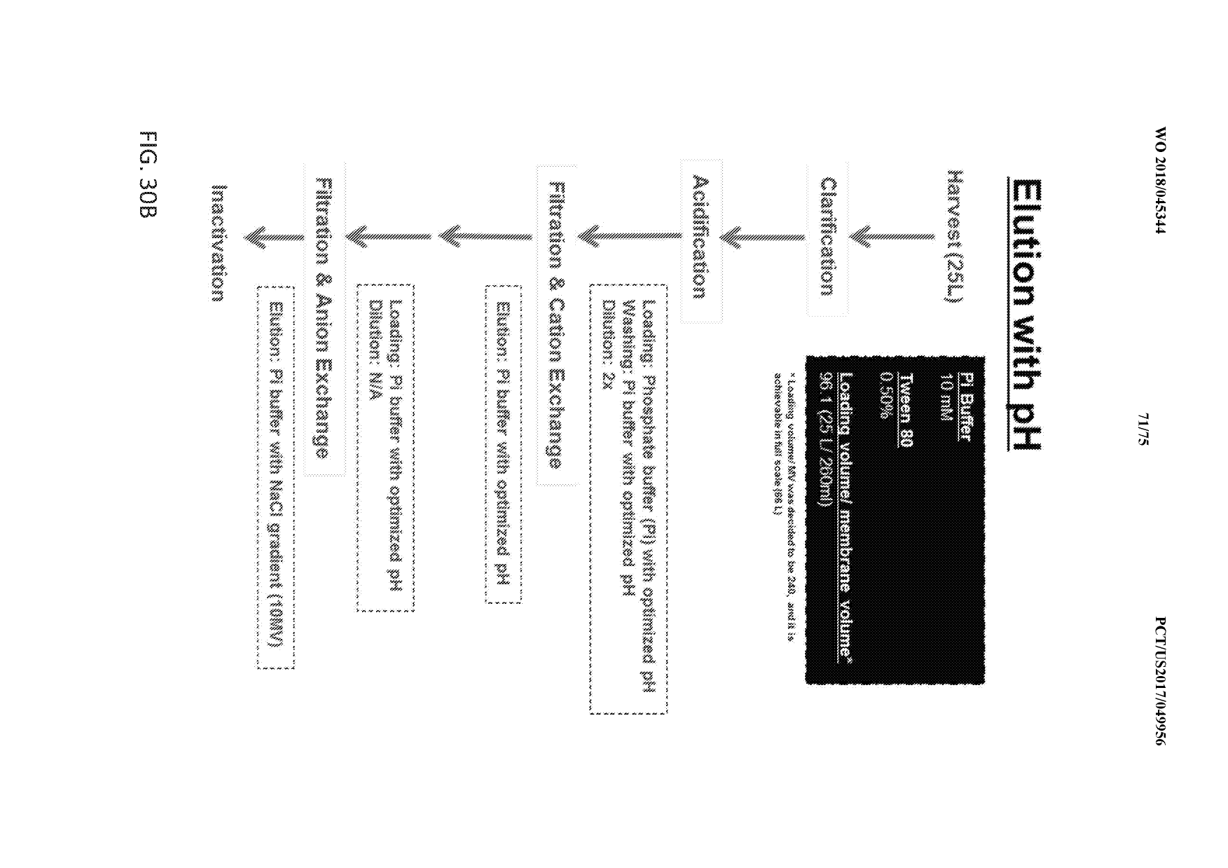

In summary, downstream processing schemes using NaCl and pH elutions are provided in FIGS. 30A & 30B, respectively.

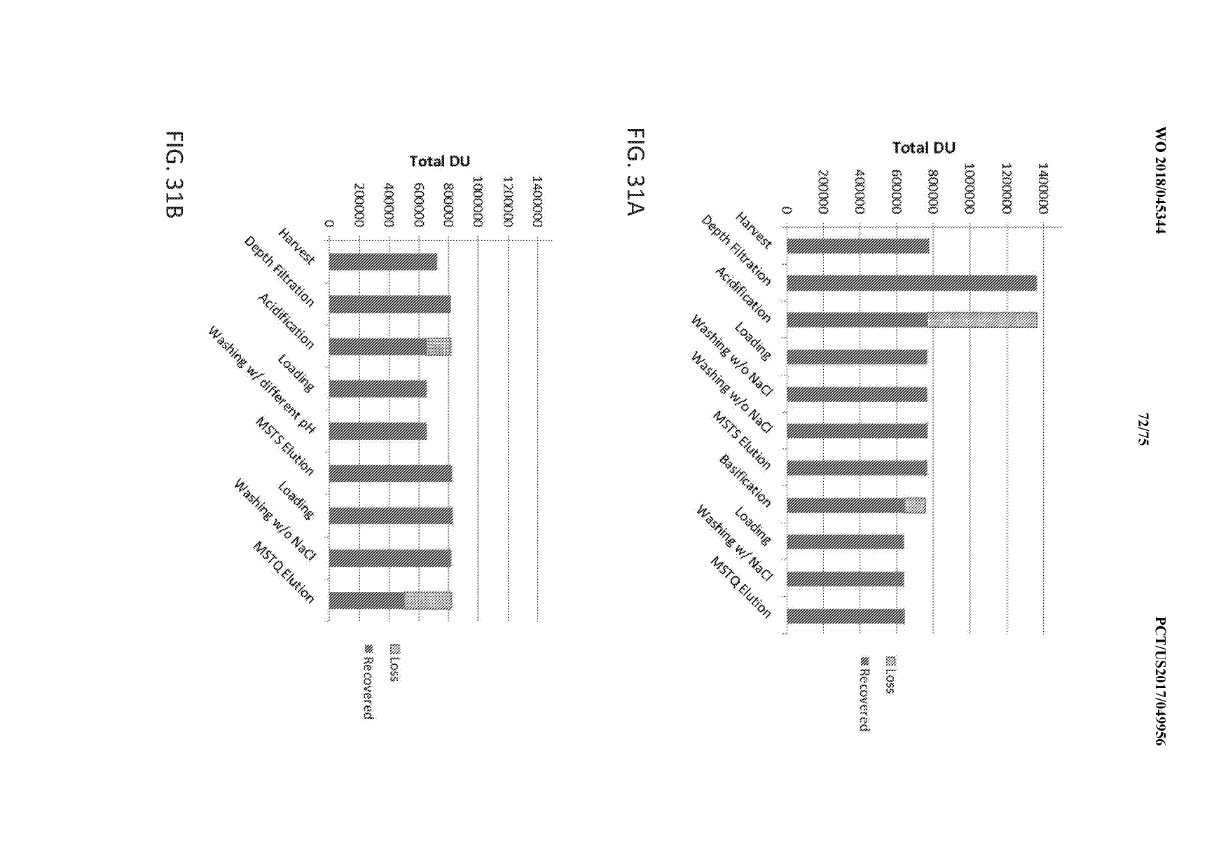

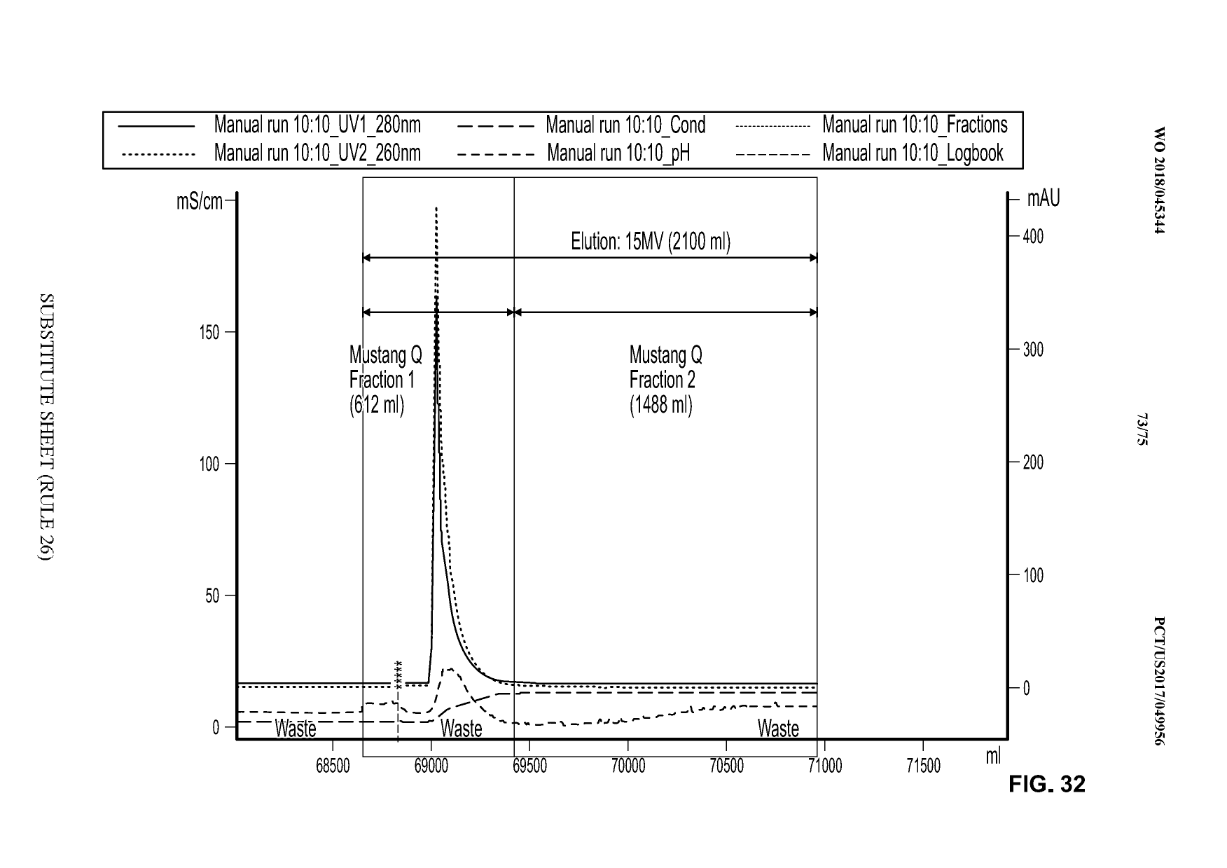

Production runs were also undertaken using poliovirus strain S2. Viral recovery at each process step of a process run using NaCl elution is summarized in FIG. 31A. Recovery and yield parameters from this run are provided in Tables I and J below. Target purity was achieved based on the total protein/DU ratio and observed concentration of BSA (cf. with Table N). Total yield from harvest to anion exchange chromatography was 81.6%. Total protein was measured using the Lowry method. BSA and host cell protein (HCP) were both measured using ELISA. Host cell DNA (HCD) was measured using real-time PCR.

TABLE I
Summary of yield and purity from S2 process run with NaCl elution.

| | | Total | Total | | | |
|---|---|---|---|---|---|---|
| Unit | | Protein | Protein/ | BSA | HCP | |
| Process | DU | (ug) | DU | (ng/ml) | (ng/ml) | HCD |
| Target | — | — | <0.1 | <10 | — | <100 |
| Harvest | 778560 | — | — | — | — | — |
| Depth | 664988 | 7639620 | 9.81 | 94.38 | 217,600 | — |
| Filtration | | | | | | |
| Mustang S | 755391 | 510900 | 0.669 | 56.02 | 48,000 | — |
| Mustang Q | 635550 | 49572 | 0.0773 | 6.35 | 38,920 | — |

TABLE J
Additional yield and purity data from S2
process run with NaCl elution.

| | | | | Total |
|---|---|---|---|---|
| Unit | | Volume | | Protein |
| Process | DU/ml | (ml) | DU | (ug) |
| Target | — | — | — | — |
| Harvest | 32 Du/ml | 24330 | 778560 | — |
| Depth | 56 Du/ml | 24330 | 1362480 | 7639620 |
| Filtration | | | | |
| Mustang S | 193.69 Du/ml | 3900 | 755391 | 510900 |
| Mustang Q | 1038.48 Du/ml | 612 ml | 635550 | 49572 |

Viral recovery at each process step of a process run using pH elution is summarized in FIG. 31B. Recovery and yield parameters from this run are provided in Tables K and L below. Target purity was achieved based on the total protein/DU ratio (cf. with Table N). BSA concentration was not determined. Total yield from harvest to anion exchange chromatography was 69.5%.

TABLE K
Summary of yield and purity from S2 process run with pH elution.

| | | Total | Total | | | |
|---|---|---|---|---|---|---|
| Unit | | Protein | Protein/ | BSA | HCP | HCD |
| Process | DU | (ug) | DU | (ng/ml) | (ng/ml) | (ng/ml) |
| Target | — | — | <0.1 | <10 | — | <100 |
| Harvest | 725000 | 482592 | 0.666 | TBA | 118,000 | 3.05 |
| | | | | | | (ADL) |
| Depth | 817000 | 470321 | 0.649 | TBA | 119,000 | 1.94 |
| Filtration | | | | | | |
| Mustang S | 826670 | 178000 | 0.271 | TBA | 131,000 | 1.91 |
| Mustang Q | 503930 | 52513 | 0.064 | TBA | 135,000 | BDL |

Abbreviations:
ADL: above detection limit;
BDL: below detection limit.

TABLE L
Additional yield and purity data from S2 process run with pH elution.

| | | | | Total |
|---|---|---|---|---|
| Unit | | Volume | | Protein |
| Process | DU/ml | (ml) | DU | (ug) |
| Target | — | — | — | — |
| Harvest | 29 Du/ml | 25000 | 725000 | 482592 |
| Depth | 32.68 Du/ml | 25000 | 817000 | 470321 |
| Filtration | | | | |
| Mustang S | 63.59 Du/ml | 1300 | 826670 | 178000 |
| Mustang Q | 359.95 Du/ml | 1400 | 503930 | 52513 |

The effect of elution volume on yield and purity of the anion exchange chromatography was next examined using the data obtained from the S2 process run with NaCl elution. As shown in Table M below, fraction 1 (see corresponding chromatograph in FIG. 32) had purity in the target range. However, when the larger fraction 2 was also included, purity was decreased and outside of the target range. These results indicate that elution volume is critical for downstream process purity. 10 mL membrane volume (MV) is thought to be sufficient based on a 60 L-scale purification.

TABLE M
Summary of yield and purity from various anion exchange fractions
from S2 process run with NaCl elution.

| | | Total | Total | | | |
|---|---|---|---|---|---|---|
| | | Protein | Protein/ | BSA | HCP | |
| Unit Process | DU | (ug) | DU | (ng/ml) | (ng/ml) | HCD |
| Target | — | — | <0.1 | <4.3 | — | <100 |
| Mustang Q | 635550 | 49572 | 0.0773 | 6.35 | 97.3 | — |
| (Fraction 1) | | | | | | |
| Mustang Q | 7812 | 58032 | 7.43 | 33.94 | 12.7 | — |
| (Fraction 2) | | | | | | |
| Mustang Q | 643362 | 107604 | 0.167 | 25.90 | 37.3 | |
| (Fraction | | | | | | |
| 1 + 2) | | | | | | |

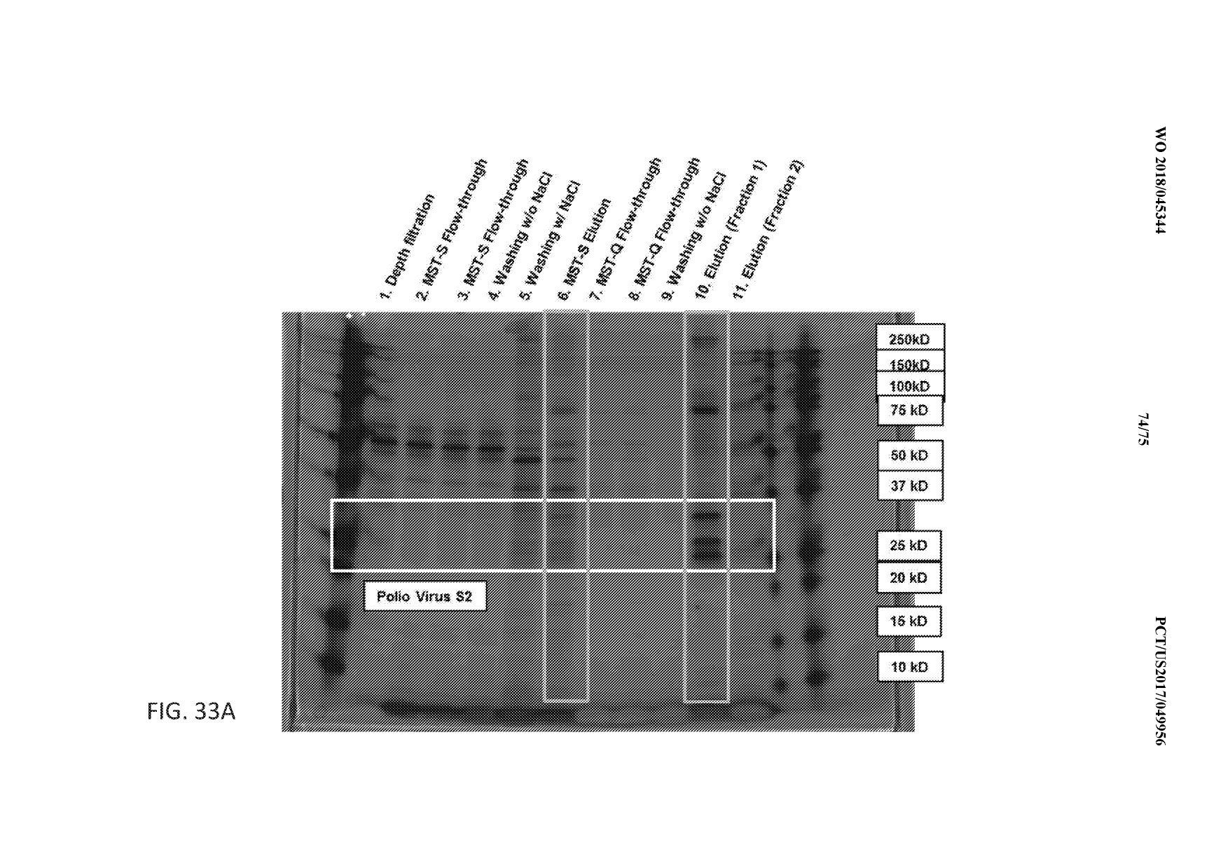

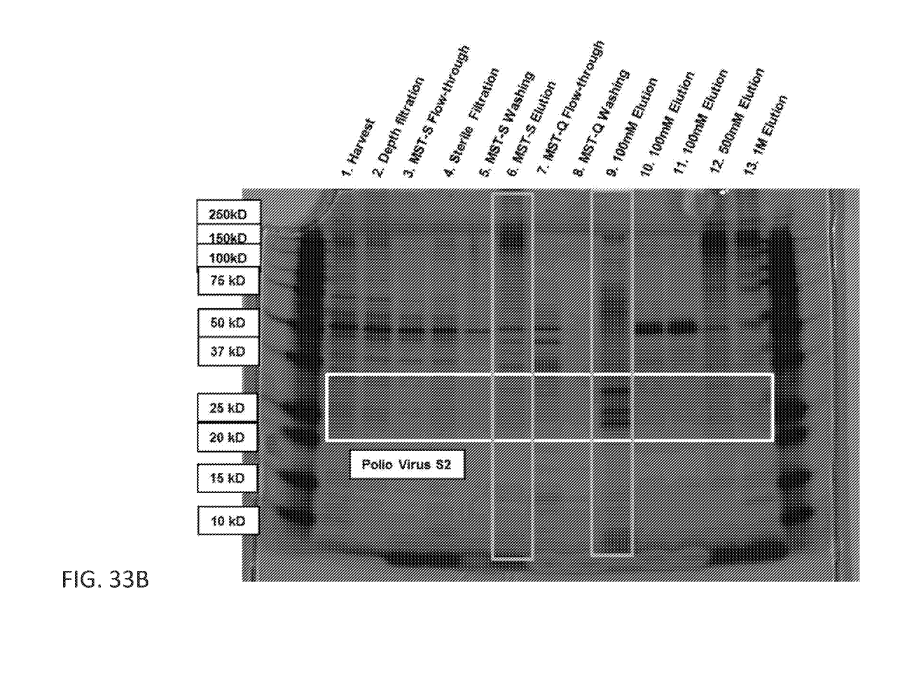

Purity of S2 virus obtained using both process runs was further analyzed by SDS-PAGE analysis. FIG. 33A shows that VP1, VP2, and VP3 were detected with higher yield in NaCl elution fraction 1 than after cation exchange chromatography. FIG. 33B shows that VP1, VP2, and VP3 were detected with higher yield in pH elution at 100 mM NaCl than after cation exchange chromatography. These results indicate that anion exchange elution was most effective with 100 mM NaCl, and that, although extra protein bands were detected by SDS-PAGE, S2 harvest is effectively purified using cation and anion exchange steps as described herein.

Target yield and purity parameters for downstream process steps are shown in Table N below.

TABLE N
Target yield and purity of downstream process.

| Items | Specification |
|---|---|
| D-antigen yield after Mustang | 60% |
| S-Q processes | (Stretched goal: 80%) |
| Total protein/D-antigen | =<0.1 ug/Du |
| Host Cell Protein | TBD |
| Host Cell DNA | <100 pg/ml |
| FBS Protein | <10 ng/ml |
| | (Total protein in FBS is 9.7 mg/1000 ml |
| | and it's 43.3% is BSA) |

Based on the results described herein, the downstream process parameters in Table O are thought to be the optimized settings for large-scale production for each poliovirus strain.

TABLE O
Downstream process parameters.

| | Process | Parameter | S1 | S2 | S3 | S3 (alt) |
|---|---|---|---|---|---|---|
| Cation | Acidification | pH | 5.7 | 5.7 | 5.0 | 5.0 |
| Exchange | Dilution | Procedure | in-line | in-line | in-line | in-line |
| | | Factor | 2-fold | 2-fold | 2-fold | 2-fold |
| | Wash | pH | 5.7 | 5.7 | 5.0 | 6.5 |
| | | NaCl conc. | 100 | 150 | 150 | 0 |
| | | (mM) | | | | |
| | Elute | pH | 5.7 | 5.7 | 5.0 | 8.0 |
| | | NaCl conc. | 250 | 250 | 300 | 0 |
| | | (mM) | | | | |
| Anion | Basification | pH | 8.5 | 8.0 | 8.5 | 8.5 |
| Exchange | Dilution | Procedure | out-line | out-line | out-line | — |
| | | Factor | 15-fold | 15-fold | 15-fold | — |
| | Wash | pH | 8.5 | 8.0 | 8.5 | 8.5 |
| | | NaCl conc. | 0 | 0 | 0 | 0 |
| | | (mM) | | | | |
| | Elute | pH | 8.5 | 8.0 | 8.5 | 8.5 |
| | | NaCl conc. | 100 | 100 | 100 | 100 |
| | | (mM) | | | | |

### Example 13: Toxicology Study in Rabbits Using sIPV Vaccine Produced by Applying the Improved Process

Toxicology studies were performed in rabbits using viral material produced from the improved methods described herein (See Example 12 above). A vaccine was formulated using viral material from S1, S2, and S3 formulated with alum and a pharmaceutically acceptable carrier. Drug substances were mixed with phosphate buffered saline containing 2-phenoxyethanol. Then the mixed solution was filtrated with a 0.2 micrometer membrane and formulated by adding alum adjuvant (alhydrogel). Sample D antigen of S1, S2, and S3 was 3, 100, and 100 DU/dose at the highest strength used.

Results

A Sabin-based inactivated poliomyelitis vaccine (sIPV) at a dose of 3:100:100 DU/dose of S1:S2:S3 was administered intramuscularly to male and female Kbl:JW rabbits once (single dose group: 2 animals/sex/group), or once each week for 5 consecutive weeks (multiple dose group: 5 animals/sex/group; dosed on Days 1, 8, 15, 22, and 29). The animals in the single dose and multiple dose groups were necropsied 2 days after the single or 5th dose, respectively, and potential toxicity was assessed. Each animal received 0.5 mL of the test article for each administration. Two concurrent control groups, physiological saline and aluminum adjuvant (vehicle) were set for each group, and administered in the same manner as the vaccinated group. An additional 5 animals/sex/group were provided to examine the reversibility of any potential toxic changes after an 8-week recovery period. It was confirmed that rabbits were the appropriate species to evaluate toxicity based on the immunogenicity results of serum samples collected in this study.

No test article related mortality was observed during the dosing and the recovery periods. One male in the vaccinated recovery group showed signs of anorexia, and was euthanized on Day 38. In this animal, a decrease in the amount of feces, a decrease in the food consumption and body weight loss were noted after the 5th dosing. Liquid supplementation was given twice; however, the animal's condition did not improve. The animal was euthanized from the standpoint of animal welfare, and a necropsy was conducted in order to elucidate the cause of the animal's deteriorating health. Findings at necropsy included a small thymus, a hairball in the stomach, and luminal dilatation of the urinary bladder. Histopathologic findings included atrophic changes in mucosa of the gastrointestinal tract (esophagus, jejunum, and ileum), epidermis of the skin, acinar cells of the lacrimal gland, lymphoid tissues in the cecum, and vacuolation of periportal hepatocytes in the liver. These findings were attributed to the animals deteriorating anorexic-like condition prior to euthanasia. Since the gastric trichobezoars are common in rabbits, and even small hairballs (or a less discrete aggregate) can cause anorexia in rabbits, the anorexic-like condition observed in this animal was likely due to the hairball found in the stomach, and was not likely to be test article related.

On the day of scheduled necropsy of the recovery group (Day 85), one female in the vaccinated recovery group was found dead without any abnormalities in clinical signs prior to death. The hematological and blood chemistry examinations conducted on Day 84, and during the dosing period (Days 2, 8, and 30), did not reveal any remarkable changes in this animal. While foreign body in the injection sites was noted microscopically, these observations were also found in other recovery animals dosed with aluminum adjuvant or the vaccine. There were no lesions attributed to the cause of death observed at necropsy or upon histopathologic examination. Although the cause of death of this rabbit was not determined, it was not considered to be test article related, since no remarkable changes were observed throughout the dosing and the recovery periods in this animal.

No test article-related abnormalities were noted in clinical signs, body weight, food consumption, water consumption, body temperature, ophthalmology, urinalysis, or hematology for the recovery periods. In the observation of skin reaction, red discoloration at the injection in the right lateral vastus muscle (injection site 3) was noted in one male in the vaccinated group from Days 30 to 34 after the 5th dosing. At necropsy of the single dose group, a dark red focus at the injection in the left lateral vastus muscle (injection site 1) (2 days after the 1st dosing) was noted in both sexes in the physiological saline, the aluminum adjuvant, and the vaccinated groups. In the multiple dose group, a dark red focus at the injection site 3 (2 days after the 5th dosing) was observed in both sexes in the aluminum adjuvant and the vaccinated groups.

Histopathological examination of the single dose group revealed mononuclear cell infiltration, necrosis/regeneration of muscle, hemorrhage, foreign body, and/or macrophage aggregation in injection site 1 (2 days after the 1st dosing) for both the aluminum adjuvant and the vaccinated groups. The incidences/severities of these changes were comparable between these groups. The foreign body was considered to be derived from the residual dose materials such as aluminum adjuvant and vaccine. In addition, the macrophage aggregation was indicative of foreign body reaction to the dose materials.

Histopathological examination of the multiple dose group revealed necrosis/regeneration of muscle, hemorrhage, foreign body, and immune/inflammatory responses such as mononuclear cell infiltration, pseudoeosinophil infiltration, edema and/or macrophage aggregation at injection site 3 in the aluminum adjuvant and vaccinated groups. Among these changes, the incidences/severities of the immune/inflammatory responses in the vaccinated group were higher than those observed in the aluminum adjuvant group. At injection site 1 (4 weeks after the 1st dosing), macrophage aggregation and foreign bodies were noted in the aluminum adjuvant and vaccinated groups, with comparable incidences/severities. Mononuclear cell infiltration and pseudoeosinophil infiltration were noted only in the vaccinated group. The incidences/severities of mononuclear and pseudoeosinophil infiltrations and foreign body in the vaccinated group decreased compared with injection site 3, which indicated the reversibility of the local immune/inflammatory changes after the injection of the vaccine. At the injection in the left sacrospinalis muscle (injection site 2) (1 week after multiple doses of the 2nd-4th dosing), mononuclear cell infiltration, pseudoeosinophil infiltration, necrosis/regeneration of muscle, foreign body, and/or macrophage aggregation were noted in the aluminum adjuvant and vaccinated groups. Among these changes, pseudoeosinophil infiltration was noted only in the vaccinated group. However, apparent exacerbation of immune/inflammatory responses or muscular necrosis was not noted in the vaccinated group, when compared to the findings after a single injection. These results indicated tolerability after multiple doses in the same region.

In addition to changes in the injection sites noted in the multiple dose group, the number and size of germinal centers of the spleen and internal iliac lymph node increased. There were also increased numbers of lymphocytes in the paracortex as well as increased pseudoeosinophil infiltration of the internal iliac lymph node. All of these changes were considered to be normal immune responses to repeated vaccination; these changes were noted in the vaccinated multiple dose group.

In the recovery period, foreign body and macrophage aggregation in the injection sites were continuously noted with comparable incidences/severities to injection site 1 after multiple doses (4 weeks after the 1st dosing); however, the other findings disappeared in the vaccinated group. Furthermore, findings in the lymph node and spleen observed at the end of the dosing period disappeared after the recovery period. Therefore, the reversibility of immune/inflammatory responses induced by vaccination was confirmed.

In conclusion, intramuscular administration of sIPV to rabbits for 4 weeks (5 doses total given at 1-week intervals) with an 8-week recovery period was well-tolerated. No systemic toxicity was observed after single dosing or multiple dosing in rabbits. Local immune/inflammatory reactions at the injection sites (and related findings in the regional lymph nodes and the spleen) were noted in the vaccinated group. However, these responses were consistent with vaccination, and the findings observed at the end of dosing period resolved during the recovery period, except for slight foreign body reaction to dose materials. A No-Observed-Effect-Level (NOEL) was not established based on the test article-related changes at injection sites: No-Observed-Adverse-Effect-Level (NOAEL) of vaccine was determined to be 0.5 mL/animal under the conditions of this study based on the reversibility of immune/inflammatory responses after the recovery period. Taken together, these experiments revealed the successful production of viral material, and subsequent formulation of an effective vaccine containing this viral material, using the improved process described herein.

1: A method for producing an Enterovirus C virus, comprising:
(a) culturing a cell in a first cell culture medium;
(b) inoculating the cell with the Enterovirus C virus in a second cell culture medium under conditions in which the Enterovirus C virus infects the cell and the infected cell produces the Enterovirus C virus; and
(c) harvesting the Enterovirus C virus produced by the cell,
wherein a surfactant is added to the second cell culture medium during inoculation of the cell with the Enterovirus C virus or from approximately one hour to approximately four hours prior to step (c).

2: The method of claim 1, wherein the yield of Enterovirus C virus harvested in step (c) is increased, as compared to a yield of Enterovirus C virus harvested in the absence of the surfactant.

3: The method of claim 1, wherein the surfactant is a polysorbate.

4: The method of claim 1, wherein the surfactant is a polyethylene glycol-based surfactant.

5: A method for producing an Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus, wherein the cell is inoculated with the Enterovirus C virus at an MOI of between about 0.01 and about 0.0009; and
(c) harvesting the Enterovirus C virus produced by the cell.

6: A method for producing an Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus, wherein between about 100,000 cells/cm² and about 300,000 cells/cm² are inoculated; and
(c) harvesting the Enterovirus C virus produced by the cell.

7: A method for producing an Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus; and
(c) harvesting the Enterovirus C virus produced by the cell,
wherein the cell is cultured during steps (a), and/or (b) at a volume/surface ratio of about 0.1 mL/cm² to about 0.3 mL/cm².

8: A method for producing an Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus, wherein the cell is inoculated at a pH that ranges from about 6.8 to about 7.4; and
(c) harvesting the Enterovirus C virus produced by the cell.

9: A method for producing an Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus, wherein no additional glucose is added to the second cell culture medium and/or glucose is depleted from the second culture medium; and
(c) harvesting the Enterovirus C virus produced by the cell.

10: A method for producing a purified Enterovirus C virus, comprising:
(a) culturing an adherent cell in a fixed bed comprising a matrix, wherein the cell is cultured in a first cell culture medium;
(b) inoculating the cell with the Enterovirus C virus in a second cell culture medium under conditions in which the Enterovirus C virus infects the cell and the infected cell produces the Enterovirus C virus;
(c) harvesting the Enterovirus C virus produced by the cell;
(d) passing the harvested Enterovirus C virus produced by the cell through a depth filter to produce a first eluate, wherein the first eluate comprises the Enterovirus C virus;
(e) binding the first eluate to a cation exchange membrane to produce a first bound fraction, wherein the first bound fraction comprises the Enterovirus C virus;
(f) eluting the first bound fraction from the cation exchange membrane to produce a second eluate, wherein the second eluate comprises the Enterovirus C virus;
(g) binding the second eluate to an anion exchange membrane to produce a second bound fraction, wherein the second bound fraction comprises the Enterovirus C virus; and
(h) eluting the second bound fraction from the anion exchange membrane to produce a purified Enterovirus C virus.

11: The method of claim 10, wherein:
(i) the depth filter has a pore size of between about 0.2 μm and about 3 μm;
(ii) before step (e) the pH of the first eluate is adjusted to a pH value of about 5.7;
(iii) a phosphate buffer or a buffer comprising polysorbate is used to bind the first eluate to the cation exchange membrane;
(iv) the first eluate is bound to the cation exchange membrane at a pH that ranges from about 4.5 to about 6.0, and wherein the first eluate is bound to the cation exchange membrane at between about 7 mS/cm and about 10 mS/cm;
(v) the first bound fraction is eluted by adjusting the pH to about 8.0 or by adding from about 0.20 M to about 0.30 M sodium chloride, and wherein the first bound fraction is eluted at between about 20 mS/cm and about 25 mS/cm;
(vi) before step (g) the pH of the second eluate is adjusted to a pH value of about 8.0 to about 8.5;
(vii) a phosphate buffer, a Tris buffer, or a the first eluate is bound to the cation exchange membrane is used to bind the second eluate to the anion exchange membrane;
(viii) the second eluate is bound to the anion exchange membrane at a pH that ranges from about 7.5 to about 8.5, and wherein the second eluate is bound to the anion exchange membrane at about 3 mS/cm; and/or
(ix) the second bound fraction is eluted by adding from about 0.05 M to about 0.10 M sodium chloride, and wherein the second bound fraction is eluted at between about 5 mS/cm and about 10 mS/cm.

12: The method of claim 1, wherein the Enterovirus C virus is a poliovirus serotype selected from the group consisting of S1, S2, and S3.

13: The method of claim 1, wherein step (b) further comprises culturing the inoculated cell under conditions in which the Enterovirus C virus infects the cell and the infected cell produces the Enterovirus C virus.

14: The method of claim 1, wherein the cell is a mammalian cell, optionally a Vero cell line is selected from the group consisting of WHO Vero 10-87, ATCC CCL-81, Vero 76 (ATCC Accession No. CRL-1587), and Vero C1008 (ATCC Accession No. CRL-1586).

15: The method of claim 1, wherein between about 4,000 cells/cm² and about 16,000 cells/cm² are cultured in step (a).

16: The method of claim 1, wherein the first cell culture medium and the second cell culture medium are different.

17: The method of claim 1, wherein the density of oxygen (DO) in the first cell culture medium during step (a) is maintained above about 50%, and wherein density of oxygen (DO) in the second cell culture medium during step (b) is maintained above about 50%.

18: The method of claim 1, wherein at least 5.0×10⁷ TCID50/mL of the Enterovirus C virus is harvested in step (d).

19: The method of claim 1, further comprising inactivating the Enterovirus C with one or more of beta-propiolactone (BPL), formalin, or binary ethylenimine (BEI).

20: An Enterovirus C virus produced by the method of claim 1, optionally wherein the virus comprises one or more antigens.


SEQUENCE LISTING (5 sequences)

SEQ ID NO: 1 - DNA, 20 bp, Artificial Sequence
  Synthetic Construct
tccatgacgttcctgacgtt

SEQ ID NO: 2 - DNA, 18 bp, Artificial Sequence
  Synthetic Construct
tctcccagcgtgcgccat

SEQ ID NO: 3 - DNA, 30 bp, Artificial Sequence
  Synthetic Construct
accgatgacgtcgccggtgacggcaccacg

SEQ ID NO: 4 - DNA, 24 bp, Artificial Sequence
  Synthetic Construct
tcgtcgttttgtcgttttgtcgtt

SEQ ID NO: 5 - DNA, 20 bp, Artificial Sequence
  Synthetic Construct
tccatgacgttcctgatgct